SEQUENCE LISTING (37 sequences)

SEQ ID NO: 1 - DNA, 870 bp, Neisseria gonorrhoeae
  source 1..870: mol_type = genomic DNA
atgaaatttatattttttgtactgtatgttttgcagtttctgccgtttgcgctgctgcacaagattgccggcctgatcggttcgcttgcctaccttctggtcaaaccgcgccgccgtatcggcgaaatcaatttggcaaaatgttttcccgaatgggacgaagaaaagcgtaaaaccgtgttgaaacagcatttcaaacacatggcaaaactgatgctcgaatacggcttatattggtacgcgtctgccaaatgcctgaaatcgctggtgcgctaccgcaataagcattatttggacgacgcgctggcggcgggggaaaaagtcatcatcctgtacccgcactttaccgcgttcgagatggcggtgtacgcgcttaatcaggatgtcccgctgatcagtatgtattcccaccaaaaaaacaagatattggacgaacagattttgaaaggccgcaaccgctatcacaacgtcttccttatcgggcgcaccgaagggctgcgcgccctcgtcaaacagttccgcaaaagcagtgcgccgttcctgtatctgcccgatcaggatttcggacgcaacaattcggtttttgtggattttttcggcattcagacggcaacgattaccggcttgagccgcattgccgcgcttgcaaatgcaaaagtgatacccgccattcccgtccgcgaggcggacaatacggttacattgcaattctatcccgcttggaaatcctttccgagtgaagacgcgcaagccgacgcgcaacgtatgaaccgctttatcgaagaacgcgtgcgcgaacacccggaacaatatttctggctgcacaagcgtttcaaaacccgtccggaaggcagccccgatttttactga

SEQ ID NO: 2 - protein, 289 aa, Neisseria gonorrhoeae
MKFIFFVLYVLQFLPFALLHKIAGLIGSLAYLLVKPRRRIGEINLAKCFPEWDEEKRKTVLKQHFKHMAKLMLEYGLYWYASAKCLKSLVRYRNKHYLDDALAAGEKVIILYPHFTAFEMAVYALNQDVPLISMYSHQKNKILDEQILKGRNRYHNVFLIGRTEGLRALVKQFRKSSAPFLYLPDQDFGRNNSVFVDFFGIQTATITGLSRIAALANAKVIPAIPVREADNTVTLQFYPAWKSFPSEDAQADAQRMNRFIEERVREHPEQYFWLHKRFKTRPEGSPDFY

SEQ ID NO: 3 - DNA, 711 bp, Neisseria gonorrhoeae
  source 1..711: mol_type = genomic DNA
atgaccaaacagctgaaattaagcgcattattcgttgcattgctcgcttccggcactgctgttgcgggcgaggcgtccgttcagggttacaccgtaagcggccaatcgaacgaaatcgtacgcaacaactatggagaatgctggaaaaacgcctactttgataaagcaagccaaggtcgcgtagaatgcggcgatgcggttgccgtccccgagcccgaacccgcgcctgtcgccgttgtggagcaggctcctcaatatgttgatgaaaccatttccctgtctgccaaaaccctgttcggtttcgataaggattcattgcgcgccgaagctcaagacaacctgaaagtattggcgcaacgcctgagtcgaaccaatgtccaatctgtccgcgtcgaaggccataccgactttatgggttctgaaaaatacaatcaggctctgtccgaacgccgcgcatacgtagtggcaaacaacctggtcagcaacggcgtacctgcttctagaatttctgctgtcggcttgggcgaatctcaagcgcaaatgactcaagtttgtcaagccgaagttgccaaactgggtgcgaaagcctctaaagccaaaaaacgtgaggctctgattgcatgtatcgaacctgaccgccgcgtagatgtgaaaatccgcagcatcgtaacccgtcaggttgtgccggcacgcaatcatcaccaacactaa

SEQ ID NO: 4 - protein, 236 aa, Neisseria gonorrhoeae
MTKQLKLSALFVALLASGTAVAGEASVQGYTVSGQSNEIVRNNYGECWKNAYFDKASQGRVECGDAVAVPEPEPAPVAVVEQAPQYVDETISLSAKTLFGFDKDSLRAEAQDNLKVLAQRLSRTNVQSVRVEGHTDFMGSEKYNQALSERRAYVVANNLVSNGVPASRISAVGLGESQAQMTQVCQAEVAKLGAKASKAKKREALIACIEPDRRVDVKIRSIVTRQVVPARNHHQH

SEQ ID NO: 5 - protein, 117 aa, synthetic construct
  REGION 1..117: note = Antibody - 2C7 VH
EVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSS

SEQ ID NO: 6 - protein, 109 aa, synthetic construct
  REGION 1..109: note = Antibody - 2C7 VL
QVVVTQESALTTSPGETVTLTCRSSTGAVTTSNYANWVQEKPDHLFTGLIGGINNRAPGVPARFSGSLIGDKAALTITGAQTEDEAIYFCALWYSNHWVFGGGTKLTVL

SEQ ID NO: 7 - protein, 10 aa, synthetic construct
  REGION 1..10: note = Anitbody - CDRH1
GYTFTDYNME

SEQ ID NO: 8 - protein, 17 aa, synthetic construct
  REGION 1..17: note = Anitbody - CDRH2
VINPNNRFTSYNQNFRG

SEQ ID NO: 9 - protein, 8 aa, synthetic construct
  REGION 1..8: note = Anitbody - CDRH3
SRWYQYDY

SEQ ID NO: 10 - protein, 14 aa, synthetic construct
  REGION 1..14: note = Anitbody - CDRL1
RSSTGAVTTSNYAN

SEQ ID NO: 11 - protein, 7 aa, synthetic construct
  REGION 1..7: note = Anitbody - CDRL2
GINNRAP

SEQ ID NO: 12 - protein, 9 aa, synthetic construct
  REGION 1..9: note = Anitbody - CDRL3
ALWYSNHWV

SEQ ID NO: 13 - DNA, 46 bp, synthetic construct
  misc_feature 1..46: note = PCR Primer
  source 1..46: mol_type = other DNA
ataggggaattgtgctcgagacgcaccacaacgacagtatggaaag

SEQ ID NO: 14 - DNA, 46 bp, synthetic construct
  misc_feature 1..46: note = PCR Primer
  source 1..46: mol_type = other DNA
tccttcagacggcattcccgggggtttctcaaagcattttggtttc

SEQ ID NO: 15 - DNA, 46 bp, synthetic construct
  misc_feature 1..46: note = PCR Primer
  source 1..46: mol_type = other DNA
ggatccccatggatacccgggcaaactatattatctgtacaaatcc

SEQ ID NO: 16 - DNA, 46 bp, synthetic construct
  misc_feature 1..46: note = PCR Primer
  source 1..46: mol_type = other DNA
aattaagtcgcgttatctagagtatcgataccgttatgaacaatcc

SEQ ID NO: 17 - DNA, 45 bp, synthetic construct
  misc_feature 1..45: note = PCR Primer
  source 1..45: mol_type = other DNA
atgccgtctgaaggatccgtcaaccgtgatatagattgaaaagtg

SEQ ID NO: 18 - DNA, 36 bp, synthetic construct
  misc_feature 1..36: note = PCR Primer
  source 1..36: mol_type = other DNA
tatccatggggatccgatccacgcgtcttaaggcgg

SEQ ID NO: 19 - DNA, 35 bp, synthetic construct
  misc_feature 1..35: note = PCR Primer
  source 1..35: mol_type = other DNA
taacgcgacttaattggccagtgtgccggtctccg

SEQ ID NO: 20 - DNA, 23 bp, synthetic construct
  misc_feature 1..23: note = PCR Primer
  source 1..23: mol_type = other DNA
tcacaattcccctatagtgagtc

SEQ ID NO: 21 - DNA, 20 bp, synthetic construct
  misc_feature 1..20: note = PCR Primer
  source 1..20: mol_type = other DNA
taatacgactcactataggg

SEQ ID NO: 22 - DNA, 21 bp, synthetic construct
  misc_feature 1..21: note = PCR Primer
  source 1..21: mol_type = other DNA
gatatccggatatagttcctc

SEQ ID NO: 23 - DNA, 21 bp, synthetic construct
  misc_feature 1..21: note = PCR Primer
  source 1..21: mol_type = other DNA
aagacatcggccggattaatc

SEQ ID NO: 24 - DNA, 23 bp, synthetic construct
  misc_feature 1..23: note = PCR Primer
  source 1..23: mol_type = other DNA
gacggtaaaaaaacggctgtcgg

SEQ ID NO: 25 - DNA, 24 bp, synthetic construct
  misc_feature 1..24: note = PCR Primer
  source 1..24: mol_type = other DNA
cttccatctcggccgagaaatacc

SEQ ID NO: 26 - DNA, 28 bp, synthetic construct
  misc_feature 1..28: note = PCR Primer
  source 1..28: mol_type = other DNA
gctctagaggtcgtctatccgttccgta

SEQ ID NO: 27 - DNA, 30 bp, synthetic construct
  misc_feature 1..30: note = PCR Primer
  source 1..30: mol_type = other DNA
cccgctcgaggggaaaggcgtgaatttgta

SEQ ID NO: 28 - DNA, 20 bp, synthetic construct
  misc_feature 1..20: note = PCR Primer
  source 1..20: mol_type = other DNA
gtgtgtccagtcgtagcagg

SEQ ID NO: 29 - DNA, 24 bp, synthetic construct
  misc_feature 1..24: note = PCR Primer
  source 1..24: mol_type = other DNA
agggatgatgataaaaccatatcc

SEQ ID NO: 30 - DNA, 21 bp, synthetic construct
  misc_feature 1..21: note = PCR Primer
  source 1..21: mol_type = other DNA
tcgtacgcaacaactatggag

SEQ ID NO: 31 - DNA, 22 bp, synthetic construct
  misc_feature 1..22: note = PCR Primer
  source 1..22: mol_type = other DNA
catcaacatattgaggagcctg

SEQ ID NO: 32 - DNA, 22 bp, synthetic construct
  misc_feature 1..22: note = PCR Primer
  source 1..22: mol_type = other DNA
ggcatttgtattttgccgtctg

SEQ ID NO: 33 - DNA, 24 bp, synthetic construct
  misc_feature 1..24: note = PCR Primer
  source 1..24: mol_type = other DNA
cgccattttctacgctttgccaag

SEQ ID NO: 34 - DNA, 15 bp, synthetic construct
  misc_feature 1..15: note = PCR Primer
  source 1..15: mol_type = other DNA
ccgcgttcgagatgg

SEQ ID NO: 35 - DNA, 18 bp, synthetic construct
  misc_feature 1..18: note = PCR Primer
  source 1..18: mol_type = other DNA
gcggaactgtttgacgag

SEQ ID NO: 36 - DNA, 20 bp, synthetic construct
  misc_feature 1..20: note = PCR Primer
  source 1..20: mol_type = other DNA
ccgccaaactcaatccttcg

SEQ ID NO: 37 - DNA, 25 bp, synthetic construct
  misc_feature 1..25: note = PCR Primer
  source 1..25: mol_type = other DNA
gcaaacttttgtttcaccgtttccg

(19) United States
(12) Patent Application Publication
(10) Pub. No.: US 2025/0186572 A1
(43) Pub. Date: Jun. 12, 2025

(54) IMMUNOGENIC COMPOSITIONS

(71) Applicant: GLAXOSMITHKLINE BIOLOGICALS SA, Rixensart (BE)

(72) Inventors: Giada BUFFI, Siena (IT); Filippo CARBONI, Siena (IT); Rossella CUFFARO, Siena (IT); Immaculada MARGARIT Y ROS, Siena (IT)

(73) Assignee: GLAXOSMITHKLINE BIOLOGICALS SA, Rixensart (BE)

(21) Appl. No.: 18/844,433

(22) PCT Filed: Mar. 7, 2023
(86) PCT No.: PCT/EP2023/055796

§ 371 Date: Sep. 6, 2024

(30) Foreign Application Priority Data
Mar. 9, 2022 (GB) 2203250.2

(51) Int. Cl. A61K 39/095; A61K 39/00; A61P 31/04

(52) U.S. Cl. CPC A61K 39/095; A61P 31/04; A61K 2039/522; A61K 2039/55505

(57) ABSTRACT

The present invention provides immunogenic compositions and vaccines comprising isolated gonococcal outer membrane vesicles (OMVs). Said isolated gonococcal OMVs display lipooligosaccharide (LOS) glycan structures comprising an oligosaccharide alpha-chain elongating from Hep I having at least four hexose monosaccharides (4Hex or 5Hex) and wherein said LOS is detoxified. The present invention relates to the use of said immunogenic compositions and vaccines in medicine and, more particularly, the use of said immunogenic compositions and vaccines in immunizing a subject against Neisseria gonorrhoeae infection.

### TECHNICAL FIELD

The present invention relates to the field of gonococcal vaccine compositions and the use of such compositions in medicine. More particularly it relates to immunogenic compositions and vaccines comprising gonococcal outer membrane vesicles (OMVs) for use in the prevention or treatment of Neisseria gonorrhoeae infection or disease.

### BACKGROUND OF THE INVENTION

Neisseria gonorrhoeae is an obligate human pathogen that causes the sexually transmitted infection (STI), gonorrhea. Gonococcal disease typically presents as a mucosal infection of the genital tract, rectum, pharynx or eye.

Neisseria gonorrhoeae contains lipooligosaccharide (LOS) as a major constituent within the outer membrane. LOS plays a key role in pathogenesis by inducing host inflammatory responses and also enabling evasion of host innate immunity through sialylation. Gonococcal LOS (structure shown in FIG. 1) is made up of three oligosaccharide chains, attached to a lipid A core. The oligosaccharide chains branch from two heptose residues attached to lipid A via two 2-keto-3-deoxy-mannooctulosonic acid (KDO) molecules. The first oligosaccharide chain elongates from a first heptose (Hep I), whilst the second and third oligosaccharide chains are connected to a second heptose (Hep II). Glycan extensions from these core heptoses (and thus the structure of the gonococcal LOS) is determined by the expression of multiple saccharide transferases, some of which are encoded in phase variable genes. This phase variability results in substantial LOS heterogeneity meaning gonococci often express several distinct LOS structures that differ in their glycan composition. These variations in structure result in heterogeneity of antigenic epitopes recognized by antibodies, as was demonstrated by the characterization of N. gonorrhoeae LOS from 20 different strains with a panel of monoclonal antibodies (Mandrell et al. Infection and immunity, 1986).

The 2C7 epitope is a conserved oligosaccharide (OS) structure, part of LOS on N gonorrhoeae, composed by two lactoses, one β-linked to Hep I, the other α-linked to Hep II. The 2C7 epitope has been examined as a potential gonococcal candidate. A peptide mimic was developed as an immunologic surrogate of the 2C7 epitope. Immunization of mice with the peptide vaccine elicited cross-reactive anti-LOS antibodies with bactericidal activity against gonococci (Gulati et al. Plos Pathog. 2003, Gulati et al. Frontiers in Immunology 2019). In literature, vaccines targeting the gonococcal LOS 2C7 epitope have been reported to be desirable in a gonococcal vaccine candidate (Gulati et al. Frontiers in immunology, 2019).

Neisseria gonorrhoeae infection is a considerable global health concern, with an estimated incidence of more than 106 million cases per year worldwide (WHO, 2018). However, because asymptomatic infections are common, the true prevalence of N. gonorrhoeae infection is not well understood. Furthermore, because of the rapid and continued emergence of antimicrobial resistance (AMR), N. gonorrhoeae has developed resistance to many antibiotics that were previously successful in treating the infection. Given the ability of gonococcus to develop AMR, a gonococcal vaccine will be key to the long-term control of N. gonorrhoeae infection and its related adverse health outcomes (Edwards J L et al. Current Op in Infectious Diseases (2018); 31(3), 246-250).

Vaccine compositions comprising outer membrane vesicles (OMV) have emerged as a promising vaccine approach for gonococcus. This is based on the observation that reduced rates of gonorrhea (31% vaccine efficacy) occurred amongst sexual health clinic patients following their vaccination with the Neisseria meningitidis serogroup B OMV vaccine, MeNZB. (Petousis-Harris H et al Lancet (2017); 390: 1603-1610). However, OMVs typically raise the best responses to homologous strains i.e., against the same strain from which they are derived (Haese et al. Vaccines 2021, 9, 804). OMV-based vaccines with improved immunogenicity against heterologous strains (i.e., strains different to the strain from which the OMVs are derived) are needed.

A further problem with gonococcal OMV based vaccines is that, due to the phase variability of the LOS biosynthesis genes it is challenging to produce a strain which produces OMVs having constant, homogenous LOS structures. Meningococcal OMV vaccines have been disclosed in which the Neisseria meningitidis used to make the blebs is engineered to fix or lock its LOS immunotype (WO2004/015099). However, the role of the LOS components of meningococcal OMV vaccines is less well understood, and the structure of meningococcal OMVs and gonococcal OMVs differ. For example, meningococcal OMVs contain a dense polysaccharide capsule which could impact the conformation and availability of meningococcal LOS epitopes (Mubaiwa et al (2017) Pathog Dis. 31; 75(5)). Furthermore, meningococcal LOS comprises a Hep II that never has an elongating Hep II-linked lactose (unlike N. gonorrhoeae) whilst a number of key virulence factors found on the surface of meningococcal OMVs are either absent in gonococcus (e.g. NadA) or are not exposed on the surface of gonococcal OMVs (e.g. FHBp) (Semchenko et al. (2019) Clin Infect Dis. 13; 69(7):1101-1111).

There remains an urgent need for an effective vaccine against Neisseria gonorrhoeae that is effective against heterologous strains.

### SUMMARY OF THE INVENTION

The present work proves a key role of antibodies targeting LOS in the bactericidal activity elicited following administration of gonococcal OMV vaccines. The inventors of the present application surprisingly discovered that gonococcal OMVs comprising LOS structures with an oligosaccharide alpha-chain extending from Hep I having at least four monosaccharides (4Hex or 5Hex), results in improved cross-strain bactericidal antibody responses, compared to gonococcal OMVs having LOS with shorter length alpha-chains (e.g. 2Hex or 3Hex) extending from Hep I. Said improved cross-bactericidal antibody response occurs regardless of whether a key LOS epitope (i.e. the 2C7 epitope) is present or absent. With this knowledge the inventors genetically engineered Neisseria gonorrhoeae strain(s) to produce a gonococcus from which OMVs that constitutively display long alpha-chain LOS structures can be obtained. A further advantage the genetic engineering (i.e., to produce a strain from which OMVs that constitutively display long alpha-chain LOS structures can be obtained) is that the gonococcus produces OMVs with consistent, homogeneous LOS structures. This is advantageous in terms of batch-to-batch consistency of the OMV-based vaccine.

Accordingly, in a first aspect there is provided an immunogenic composition comprising a population of isolated gonococcal outer membrane vesicles (OMVs) said isolated gonococcal OMVs displaying lipooligosaccharide (LOS) glycan structures comprising oligosaccharide alpha-chains elongating from Hep I having at least four hexose monosaccharides (4Hex or 5Hex), wherein the isolated gonococcal OMVs are obtained or obtainable from a genetically modified Neisseria gonorrhoeae strain comprising genetic modifications that
a) renders the phase variability of at least one LOS glycosyl transferase (lgt) gene(s) non-phase variable, and
b) detoxify the LOS
wherein the population of isolated gonococcal OMVs comprise a higher content of LOS glycan structures comprising at least four hexose monosaccharides (4Hex or 5Hex) than that of a comparator population of isolated gonococcal OMVs, the comparator population of isolated gonococcal OMVs being obtained or obtainable from a Neisseria gonorrhoeae bacterium that comprises phase variable lgt genes.

In a further aspect there is provided an immunogenic composition comprising isolated gonococcal OMVs obtained or obtainable from a genetically modified Neisseria gonorrhoeae bacterium said bacterium comprising genetic modification(s) that
a) render the phase variability of at least one lipooligosaccharide glycosyl transferase (lgt) gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and,
b) detoxify the LOS

In a further aspect there is provided an immunogenic composition comprising isolated gonococcal outer membrane vesicles (OMVs) wherein said composition substantially comprises OMVs that display lipooligosaccharide (LOS) glycan structures comprising an oligosaccharide alpha-chain elongating from Hep I having at least four hexose monosaccharides (4Hex or 5Hex) and wherein said LOS is detoxified.

In a further aspect there is provided a vaccine comprising the immunogenic compositions.

In a further aspect there is provided the immunogenic compositions or the vaccine for use in medicine.

In a further aspect there is provided the immunogenic compositions or the vaccine for use in the prevention or treatment of Neisseria gonorrhoeae infection or disease.

In a further aspect there is provided the immunogenic compositions or the vaccine for use in immunizing a subject against N. gonorrhoeae infection.

In a further aspect there is provided the immunogenic compositions or the vaccine for use in generating a bactericidal immune response.

In a further aspect there is provided the immunogenic compositions or the vaccine, for use in inducing antibodies that are bactericidal against N. gonorrhoeae

In a further aspect there is provided a method for the treatment or prevention of disease caused by N. gonorrhoeae in a subject in need thereof, said method comprising administering to said subject a therapeutically effective amount of the immunogenic compositions or the vaccine.

In a further aspect there is provided a method for immunizing a subject in need thereof against N. gonorrhoeae, comprising administering to the subject an immunologically effective amount of the immunogenic compositions or the vaccine.

In a further aspect there is provided a method for raising an immune response in a subject, comprising administering the immunogenic compositions or the vaccine to the subject.

In a further aspect there is provided the use of the immunogenic compositions or the vaccine in the manufacture of a medicament for the treatment or prevention of disease caused by Neisseria gonorrhoeae.

In a further aspect there is provided the immunogenic composition or vaccine for use, the method or the use, wherein at least 2 doses of the composition are administered to a subject.

In a further aspect there is provided the immunogenic composition or vaccine for use, the method or the use wherein the subject is at increased risk of infection with N. gonorrhoeae relative to the average risk in the general population.

In a further aspect there is provided the immunogenic composition or vaccine for use, the method or the use wherein the subject is co-immunized against one or more further infectious agents.

### BRIEF DESCRIPTION OF THE FIGURES

FIG. 1: Example of a complete LOS structure.

FIG. 2: Schematic representation of LOS glycan extensions from the core heptoses highlighting the epitopes targeted by the 2C7, L1, 4C4 and L3,7,9 mAbs.

FIG. 3: Schematic representation of the different LOS structures exposed on the outer membrane (and OMVs) of the eight MS11 lgt mutant strains (2HexG+, 2HexG−, 3HexG+, 3HexG−, 4HexG+, 4HexG−, 5HexG+, 5HexG−).

FIG. 4: Silver stained SDS-PAGE and western blot with four different LOS specific mAbs of OMVs from MS11 LOS mutant strains (2HexG+, 2HexG−, 3HexG+, 3HexG−, 4HexG+, 4HexG−, 5HexG+, 5HexG−).

FIG. 5: HILIC-MS profiles of OMVs from MS11 LOS mutant strains. Two profiles are provided, the first of which shows the profile of the G− structures (2HexG−, 3HexG−, 4HexG− and 5HexG−), the second of which shows the profile of the G+ structures (2HexG+, 3HexG+, 4HexG+ and 5HexG+).

FIG. 6: hSBA titers measured against the indicated gonococcus strains on pooled sera from CD1 mice immunized twice with OMVs obtained from three different OMV vaccine compositions or an Alum control.

FIG. 7: hSBA titers measured against the indicated gonococcus strains on pooled sera from CD1 mice immunized twice with OMVs (referred to as GMMA in FIG. 7) obtained from MS11 lgt mutant strains (2HexG+, 2HexG−, 3HexG+, 3HexG−, 4HexG+, 4HexG−, 5HexG+, 5HexG). The MS11 lgt mutant strains also contained a Δlpxl1 mutation.

FIG. 8: hSBA titers measured against the indicated gonococcus strains using a pool of sera from mice immunized twice with OMVs obtained from an FA 1090 2KO (Δlpxl1,Δrmp) strain after incubation with 3 concentrations of each competitor: LOS extracted from wild-type FA1090, OMVs obtained from an FA1090-1KO (Δlpxl1) strain including an ΔLgtF mutation and, OMVs obtained from an FA1090 2KO (Δlpxl1,Δrmp) strain lot 32. SBA buffer was used as the “not inhibited” control sample.

FIG. 9: hSBA titers measured against the indicated gonococcus strains using a pool of sera from mice immunized twice with OMVs obtained from an FA1090 2KO (Δlpxl1,Δrmp) strain after incubation with 3 concentrations of each competitor: OMVs from Δlpxl1 MS11 LOS mutant strains (OMV 2HexG−, OMV2HexG+ OMV 3HexG−, OMV 3HexG+ OMV 4HexG−, OMV 4HexG+ OMV 5HexG−, OMV 5HexG+) and SBA buffer that represent the “not inhibited” sample.

FIG. 10A: The structure of lipid A corresponding to the hexa-acylated form.

FIG. 10B: The structure of lipid A corresponding to the penta-acylated form purified from genetically modified Neisseria gonorrhoeae comprising an Δlpxl1 mutation.

FIG. 11: Silver stain and western Blots to determine LOS structures present on OMVs obtained from an FA1090 strain gonococcus with a single (1KO) Δlpxl1 mutation. Reference to GMMA/TRD numbers represent different lots of OMV preparations. The horizontal line present in the silver stain is representative of the height of the 3.5 kDa marker.

FIG. 12: Silver stain and western Blots to determine LOS structures present on OMVs obtained from an FA1090 strain gonococcus with a double (2KO) Δlpxl1, Δrmp mutation. Reference to GMMA/TRD numbers represent different lots of OMV preparations. The horizontal line present in the silver stain is representative of the height of the 3.5 kDa marker.

FIG. 13: Western blots with four different LOS specific mAbs of OMVs isolated from F62 mutant strains 1KO (Δlpxl1) and 2KO (Δlpxl1, Δrmp) lots.

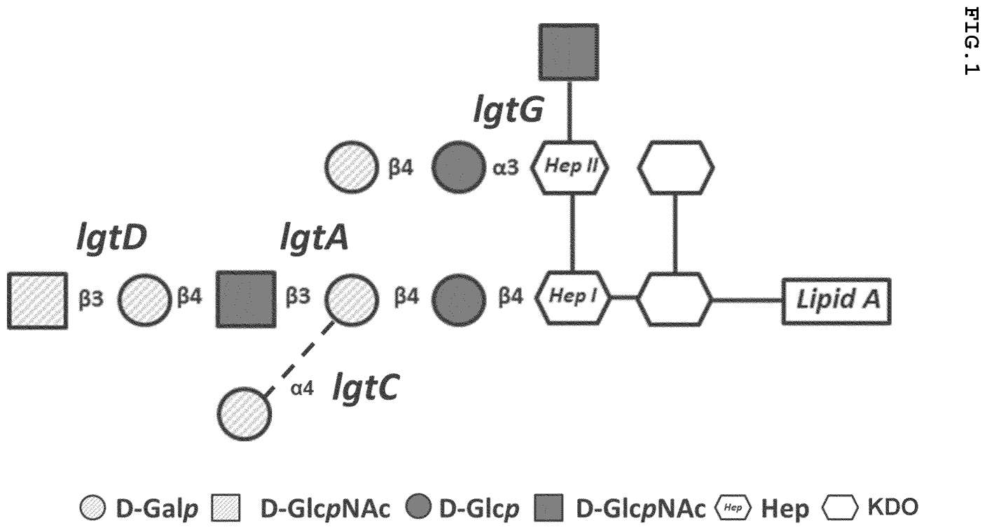

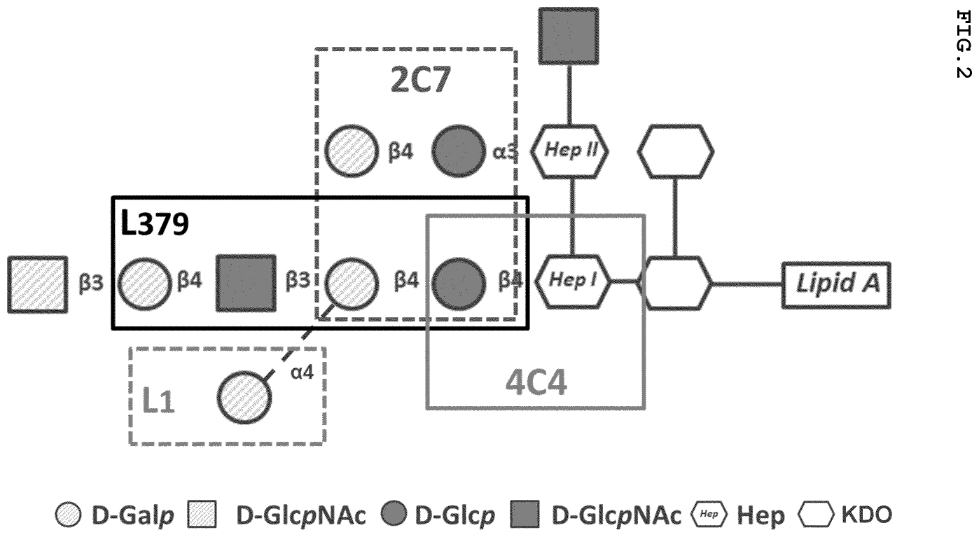

### DETAILED DESCRIPTION OF THE INVENTION

Prior to setting forth the invention in detail, it may be helpful to the understanding of one of ordinary skill to define the following terms:

Unless otherwise explained or defined herein, all technical and scientific terms used herein have the same meaning as commonly understood by one of ordinary skill in the art to which this disclosure belongs. For example, definitions of common terms in molecular biology can be found in Benjamin Lewin, Genes V, published by Oxford University Press, 1994 (ISBN 0-19-854287-9); Kendrew et al. (eds.), The Encyclopaedia of Molecular Biology, published by Blackwell Science Ltd., 1994 (ISBN 0-632-02182-9); and Robert A. Meyers (ed.), Molecular Biology and Biotechnology: a Comprehensive Desk-Reference, published by VCH Publishers, Inc., 1995 (ISBN 1-56081-569-8).

All references, including publications of patent and or patent applications cited within this patent specification are incorporated by reference herein.

The articles “a” and “an” are used herein to refer to one or to more than one (i.e., to at least one) of the grammatical object of the article. By way of example, “an element” means one element or more than one element.

“Or” supports, contemplates, and when recited in the claims, claims “one or a combination of” as in “one or a combination of A, B, or C.” To illustrate, “A, B, or C” means A alone, B alone, C alone, the combination of A and B, the combination of A and C, the combination of B and C, and the combination of A, B, and C, unless otherwise illustrated. That is, “or” supports and contemplates “and” as in “and/or” wherein “and/or” includes any combinations within the list of alternatives without being limited solely to the combination of all alternatives in a list (i.e. “A, B, or C” includes “A and B” and is not limited to “A, B, and C”).

Furthermore, the recitation of a list of alternatives, which may be conjoined by “and” and from which at least one alternative is selected, further contemplates and supports all combinations within the list of alternatives. For example, “X is selected from the group of: A, B, and C” contemplates and supports “X is selected from the group of: A, B, C, and combinations thereof,” “X is selected from at least one of the group of: A, B, and C,” and “X is selected from one or more of the group of A, B, and C.” For further example, “X is selected from the group consisting of A, B, and C” contemplates and supports “X is selected from the group consisting of A, B, C, and combinations thereof,” “X is selected from at least one of the group consisting of A, B, and C,” or “X is selected from one or more of the group consisting of A, B, and C.”

Each of the following contemplates and supports any of the others: “comprises,” “consists of,” “consists essentially of,” “is/are/being,” “is selected from,” “is at least selected from,” “is selected from the group of,” “is selected from the group consisting of,” “is at least selected from the group consisting of,” “is from at least one of the group consisting of,” and “is from one or more of the group consisting of.” For example and in consideration of the above regarding combinations of listed elements, recitation of “X comprises an A, a B, or a C” in the specification contemplates and supports embodiments wherein “X consists of an A, a B, or a C,” “X consists of an A, a B, a C, or combinations thereof,” “X consists of one or more of an A, a B, or a C,” “X is one or more of an A, a B, or a C,” “X is an A, a B, a C, or combinations thereof,” “X is selected from an A, a B, or a C,” “X is selected from an A, a B, a C, or combinations thereof,” “X is selected from the group consisting of an A, a B, a C, and combinations thereof,” “X is selected from at least one of the group consisting of an A, a B, and a C,” or “X is selected from one or more of the group consisting of an A, a B, and a C.”

When a specific component of an embodiment is listed—e.g. “X comprises A, B, or C”-then also supported and contemplated are any embodiments which specifically exclude any individual or combinations of components—e.g. “X comprises A, but not B or C” or “X comprises A but does not comprise B or C.” [00048]“About” as used herein when referring to a measurable value such as an amount, a temporal duration, a quantum of measurement, and the like, is meant to encompass variations of .+−0.20% or .+−0.10%, more preferably .+−0.5%, even more preferably .+−0.1%, and still more preferably .+−0.0.1% from the specified value, as such variations are appropriate to perform the disclosed methods.

“Sequence,” “segment,” “nucleic acid,” or “region” as used within the context of a nucleic acid includes sense (i.e. positive) and anti-sense (i.e. negative, e.g. reverse complementary) sequences of the same nucleic acid. A “segment,” “sequence,” “nucleic acid,” or “region” that “encodes” a coding sequence, wherein the coding sequence is transcribed and/or translated, includes sense and antisense (e.g. reverse complementary) sequences of the same nucleic acid.

To illustrate how “sequence,” “segment,” “nucleic acid,” or “region” as used within the context of a nucleic acid includes sense (i.e. positive) and anti-sense, if a specific sequence, called “A”, is listed as having the sequence of 5′-ATGG-3′ in the sense strand (i.e. positive strand) then it is contemplated, supported, and when listed in the claims, claimed that A also has the sequence of 3′-TACC-5′ in the antisense strand (i.e. negative strand) or complementary strand (i.e. A comprises 5′-ATGG-3′ or 3′-TACC-5′).

“Sequence,” “region,” or “segment” as used herein, unless otherwise specified, also contemplates and supports sequences incorporating different forms of nucleic acids, i.e. RNA and DNA, of the same information, or sequences incorporating differing nucleotides found in the different forms of the nucleic acids (i.e. uridines in RNA and thymidines in DNA), as well as sense and anti-sense (e.g. reverse complementary) information therein. To illustrate, if A in RNA (sense) is 5′-AUGG-3′, A also comprises 5′-ATGG-3′, being the sense DNA, and 3′-TACC-5′ being the anti-sense DNA, as well as 3′-UACC-5′, being the antisense RNA. To distinguish between sense and anti-sense (e.g. complementary) sequences, a prime symbol (′) may be used, i.e. for ease of tracking original genomic material, transcripts, first strand synthesis, second strand synthesis, sense, and antisense strands.

As used herein, the abbreviation WT corresponds to “wild-type”.

As used herein the term “isolated gonococcal OMVs” means outer membrane vesicles (OMVs) that have been the subject of an isolation or extraction procedure and have thus been removed from their natural environment, their natural environment being either i) the outer membrane of the gonococcal bacterium or b) the cell-free supernatant following natural secretion from the bacterial cell surface.

As used herein the term “genetic modification(s)” means any alteration to the constitution, structure or operation of the genetic material in a cell to provide a specified effect (e.g. decreasing or abolishing expression or rendering the expression of a gene or gene(s) non-phase variable). Genetic modification(s) are made from to a starting organism which is either non-modified (e.g. wild-type) and/or is modified but for comprising a previously unmodified (e.g. wild-type) gene of interest. The genetic material within a cell relates to either DNA or RNA. As such, the term genetic modification as used herein, means any artificial alteration to the constitution, structure or operation of either gonococcal DNA or RNA such as to decrease and/or abolish expression and/or function of the specified gene(s) or to render the specified gene(s) non-phase variable. As used herein, “genetically modified” with regards to gonococcal bacterium refers to a gonococcus that has had its genetic material artificially altered. Genetically modified gonococcal bacteria do not include wild-type gonococcal bacteria. A genetically modified gonococcal bacterium includes for example a gonococcal bacterium into which an exogenous polynucleotide has been introduced. A genetically modified gonococcal bacterium also refers to a bacterium that has been genetically manipulated such that endogenous nucleotides have been altered to include a mutation, such as a deletion, an insertion, a substitution or a combination thereof. For instance, an endogenous coding and/or non-coding region could be deleted or replaced. Such genetic modifications may result either in depleted and/or abolished expression of a polypeptide and/or may result in a polypeptide having a different amino acid sequence than was encoded by the endogenous polynucleotide. Such genetic modifications may result in the gene no longer being phase variable or phase variably expressed (i.e. rendering the expression of a gene or genes, non-phase variable). For example, a genetically modified gonococcal bacterium may have an altered regulatory sequence, such as a promotor, to result in increased or decreased expression of an operably linked endogenous coding region.

As used herein the term “gene deletion” or “gene knockout” refers to a combination of genetic techniques that has the potential to render a specific gene inoperable or inactive. In some embodiments a gene deletion decreases or abolishes expression of a polypeptide from the gene. In some embodiments both the mRNA and protein are reduced or eliminated. In certain embodiments the expression of gene is substantially decreased or abolished. Substantially decreased means that the expression of a gene is reduced by at least 70%, at least 80%, at least 90%, at least 95% or at least 98% when compared to an endogenous level of expression of a gene. In certain embodiments the expression of a gene is abolished. Abolished means that, using techniques to monitor the expression of either the mRNA transcribed from a gene, or the expression of protein translated from a particular mRNA, no level of detection is observed. Expression of a gene can be determined by a suitable technique (e.g., by measuring transcript levels by RT/Q-PCR or expressed protein levels by immunoassay e.g. western blot). Gene deletion or gene knockout might include not only deletion of genetic elements but also addition, substitution or modification, such that the gene is inoperable or inactive, i.e. insertion of a genetic sequence may cause mistranslation of the gene, by for example, incorporating an early stop codon, or by causing a missense translation. Genes may for example be deleted by replacement of the gene, or a fragment of said gene, with a different heterologous gene (e.g. an antibiotic resistance gene) for example by homologous recombination.

As used herein the “A” symbol is used herein to refer to a bacterial strain from which the sequence of the gene recited after the A symbol has been deleted/knocked out in line with the definition of “gene deletion” or “gene knockout”.

As used herein, the term “phase variable” means that the expression of a specified gene may reversibly be switched on and off, resulting in the expression of phenotypes that vary within a clonal population. As used herein the term “non-phase variable” or “rendering a gene non-phase variable” means that gene(s) (e.g. the lipooligosaccharide glycosyl transferase (lgt) genes) that were previously phase variable, are rendered not susceptible to phase variation beyond the background chance of a non-site-specific switching on or off of functional gene expression.

Reference to “lipooligosaccharide” (or LOS) may also be referred to as “lipopolysaccharide” (or LPS).

Amino acids refers to an amino acid selected from the group consisting of alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), valine (val, V).

A “subject” as used herein is an animal, preferably a mammal, including humans, non-human primates and non-primate mammals such as members of the rodent genus (including but not limited to mice and rats), the Cavia genus (including but not limited to guinea pigs) and members of the order Lagomorpha (including but not limited to rabbits). As used herein, the subject is most preferably a human.

As used herein, “immune response” means the sequence of events occurring at the molecular, cellular or tissue level (i.e. at any level of biological organisation) in response to an antigen. In the context of the present disclosure, “immune response” may be the sequence of cellular (cell mediated) and/or humoral (antibody mediated) events occurring in response to an antigen (e.g. antigens on the surface of bacteria, viruses, fungi etc.) or in response to antigens present on the surface of an OMV or antigens in the form of an immunogenic fragment, immunogenic composition or vaccine. As used herein, “immunogenicity” means the ability of an antigen to elicit an immune response.

As used herein, “adjuvant” means a compound or substance (or combination of compounds or substances) that, when administered to a subject in conjunction with an antigen or antigens, for example as part of an immunogenic composition or vaccine, increases or enhances the subject's immune response to the administered antigen or antigens, compared to the immune response obtained in the absence of adjuvant. With respect to the present disclosure, the adjuvant may additionally mean a compound or substance (or combination of compounds or substances) that, when administered to a subject in conjunction with outer membrane vesicles, for example as part of an immunogenic composition or vaccine, increases or enhances the subjects immune response to the antigens present on the surface of the outer membrane vesicle and/or functions to decrease the reactogenicity of the outer membrane vesicle.

As used herein the term “immunogenic composition” relates to a composition of matter suitable for administration to a human or animal subject (e.g., in an experimental or clinical setting) that is capable of eliciting a specific immune response, e.g., against a pathogen, such as Neisseria. As such, an immunogenic composition includes one or more antigens (for example, polypeptide antigens) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject, wholly or partially, against symptoms or conditions induced by a pathogen. In the context of this disclosure, the term immunogenic composition will be understood to encompass compositions that are intended for administration to a subject or population of subjects for the purpose of eliciting a protective immune response against Neisseria gonorrhoeae.

By “immunologically effective amount”, it is meant that the administration of that amount to an individual, either in a single dose or as part of a series, is effective for treatment, protection or prevention. Administration of an immunologically effective amount elicits an immune response, including a protective immune response. This amount can vary depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range.

As used herein the term “outer membrane vesicle(s)” or “OMV(s)” relates to proteoliposomic vesicles obtained by disruption of, or blebbing from the outer membrane of Gram-negative bacteria, to form vesicles that retain antigens from the outer membrane. Heterologous antigens are expressed in the Gram-negative bacteria such that they assemble in the membrane that is then released into the culture supernatant. OMVs from such bacteria are representative of the outer membrane and periplasmic bacterial compartments and allow the presentation of membrane proteins in their natural composition and structure. In the broadest sense, OMVs relates to any such proteoliposomic vesicles. The term OMVs includes ‘Native OMVs’ (nOMVs), microvesicles (MVs), and detergent-extracted OMVs (dOMVs). All of these form are collectively referred to as OMVs herein, unless otherwise specifically mentioned. In a preferred embodiment, the OMVs are nOMVs. As used herein the term outer membrane vesicle(s) or OMV's may also be referred to as Generalised Modules for Membrane Antigens (GMMA).

As used herein the term “Lipooligosaccharide” (LOS) may also be referred to as (or used interchangeably with) the term lipopolysaccharide (LPS).

As used herein the term “alpha-chain” refers to the Hep I-linked oligosaccharide chain. As used herein the term “beta-chain” refers to the Hep-JI linked oligosaccharide (or disaccharide) chain. The heterogeneity of the LOS includes heterogeneity in the number of sugars extending from both Hep I (alpha-chain) and Hep II (beta-chain). As used herein, the number of sugars extending from the alpha-chain and beta-chain are described using the identifiers described in Table 1 below. The disclosed identifiers include a number (e.g. 2, 3, 4 or 5) which corresponds to the number of monosaccharides that extend from Hep I on the alpha-chain. The disclosed identifiers further include either a G+ or a G− as follows:
G+ indicates the presence of a disaccharide chain extending from Hep II (i.e. beta-chain) or,
G− that indicates the absence of said beta-chain.
As used herein, reference to the number of monosaccharides that extend from Hep I on the alpha-chain (2Hex, 3Hex, 4Hex or 5Hex), along with an absence ofany reference to the presence or absence of the beta-chain (i.e. absence of G−/G+) indicates that the beta-chain may either be present or absent.

TABLE 1
LOS Identifiers

| Identifier | LOS structure | LOS structure (simplified) |
|---|---|---|
| 2HexG− | β-Gal-(1→4)-β-Glc-(1→4)-Hep I | Gal-Glc-Hep I |
| | Unsubstituted Hep II | Unsubstituted Hep II |
| 2HexG+ | β-Gal-(1→4)-β-Glc-(1→4)-Hep I | Gal-Glc-Hep I |
| | β-Gal-(1→4)-α-Glc-(1→3)-Hep II | Gal-Glc-Hep II |
| 3HexG− | α-Gal-(1→4)-β-Gal-(1→4)-β-Glc-(1→4)-Hep I | Gal-Gal-Glc-Hep I |
| | Unsubstituted Hep II | Unsubstituted Hep II |
| 3HexG+ | α-Gal-(1→4)-β-Gal-(1→4)-β-Glc-(1→4)-Hep I | Gal-Gal-Glc-Hep I |
| | β-Gal-(1→4)-α-Glc-(1→3)-Hep II | Gal-Glc-Hep II |
| 4HexG− | β-Gal-(1→4)-β-GlcNAc-(1→3)- β-Gal- | Gal-GlcNAc-Gal-Glc-Hep I |
| | (1→4)-β-Glc-(1→4)-Hep I | Unsubstituted Hep II |
| | Unsubstituted Hep II | |
| 4HexG+ | β-Gal-(1→4)-β-GlcNAc-(1→3)- β-Gal- | Gal-GlcNAc-Gal-Glc-Hep I |
| | (1→4)-β-Glc-(1→4)-Hep I | Gal-Glc-Hep II |
| | β-Gal-(1→4)-α-Glc-(1→3)-Hep II | |
| 5HexG− | β-GalNAc-(1→3)-β-Gal-(1→4)-β-GlcNAc- | GalNAc-Gal-GlcNAc-Gal-Glc-Hep I |
| | (1→3)- β-Gal-(1→4)-β-Glc-(1→4)-Hep I | Unsubstituted Hep II |
| | Unsubstituted Hep II | |
| 5HexG+ | β-GalNAc-(1→3)-β-Gal-(1→4)-β-GlcNAc- | GalNAc-Gal-GlcNAc-Gal-Glc-Hep I |
| | (1→3)- β-Gal-(1→4)-β-Glc-(1→4)-Hep I | Gal-Glc-Hep II |
| | β-Gal-(1→4)-α-Glc-(1→3)-Hep II | |

### Immunogenic Composition

In a first aspect there is provided an immunogenic composition comprising isolated gonococcal OMVs obtained or obtainable from a genetically modified Neisseria gonorrhoeae bacterium said bacterium comprising genetic modification(s) that a) render the phase variability of at least one lipooligosaccharide glycosyl transferase (lgt) gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS. In some embodiments and as a result of said genetic modification(s), the immunogenic composition comprises isolated gonococcal OMVs displaying LOS glycan structures comprising an oligosaccharide alpha-chain elongating from Hep I having at least four hexose monosaccharides (4Hex or 5Hex). In some embodiments, the immunogenic compositions comprise OMVs displaying LOS glycan structures comprising an oligosaccharide alpha-chain elongating from Hep I having at least four hexose monosaccharides (4Hex or 5Hex) including any of 4HexG−, 4HexG+, 5HexG- or 5HexG+ LOS glycan structures. In an embodiment, the immunogenic compositions comprise isolated gonococcal OMVs obtained or obtainable from a genetically modified Neisseria gonorrhoeae bacterium said bacterium comprising genetic modification(s) that render the phase variability of at least one lgt gene(s) non-phase variable thus resulting in a bacterium that produces OMVs displaying 4HexG−4HexG+, 5HexG− or 5HexG+ LOS glycan structures.

In some embodiments, the starting organism to which the genetic modification(s) is/are then introduced is a substantially or completely unmodified gonococcal bacterium. As such provided is a genetically modified gonococcal bacterium, comprising genetic modification(s) that: a) render the phase variability of at least one lgt gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS, wherein the unmodified gonococcal bacterium is a wild-type gonococcal bacterium. In some embodiments, the starting organism is not a wild-type gonococcal bacterium but is a gonococcal bacterium that comprises no genetic modification(s) to either the lgt genes or to any genes that result in a detoxified LOS or a LOS with reduced endotoxin activity.

In some embodiments, the starting organism to which a genetic modification(s) is introduced is a substantially or completely unmodified gonococcal bacterium of any strain. Accordingly, the starting organism to which a genetic modification(s) is then introduced is a wild-type gonococcal bacterium of any strain or is a gonococcal bacterium of any strain comprising no genetic modification(s) to either the lgt genes or to any genes that result in a detoxified LOS or a LOS with reduced endotoxin activity. In an embodiment, the genetically modified Neisseria gonorrhoeae bacterium is derived from strain MS11, BG27, BG8, F62, FA1090, WHO-F, WHO-M, WHO-N, WHO-G, GC14, SK92-679 or GC_0817560. In an embodiment, the starting organism to which a genetic modification(s) is then introduced is a substantially or completely unmodified gonococcal bacterium of strain MS11, BG27, BG8, F62, FA1090, WHO-F, WHO-M, WHO-N, WHO-G, GC14, SK92-679, or GC_0817560. In an embodiment, the unmodified strain is not FA1090. In an embodiment, the unmodified strain is not MS11.

In an embodiment, said genetic modification(s) that render the phase variability of at least one lgt gene(s) non-phase variable results in a bacterium that is incapable of producing OMVs displaying LOS glycan structures comprising an oligosaccharide alpha-chain elongating from Hep I having less than four hexose monosaccharides (2Hex or 3Hex). A bacterium that is described as being “incapable of producing OMVs displaying LOS glycan structures comprising an oligosaccharide alpha-chain elongating from Hep I having less than four hexose monosaccharides (2Hex or 3Hex)” means a bacterium that substantially does not express (or produce OMVs that display) LOS having short oligosaccharide alpha-chain (2Hex or 3Hex) structures beyond the background chance of such expression. In an embodiment, less than 20%, less than 15%, less than 10%, less than 9%, less than 8%, less than 7%, less than 6%, less than 5%, less than 4%, less than 3%, less than 2% or less than 1% of the total LOS expressed by the bacterium (or displayed on OMVs obtained therefrom) comprises oligosaccharide alpha-chain elongating from Hep I having less than four hexose monosaccharides (2Hex or 3Hex).

In an embodiment, there is provided an immunogenic composition comprising isolated gonococcal OMVs obtained or obtainable from a genetically modified Neisseria gonorrhoeae bacterium said bacterium comprising genetic modification(s) that a) render the phase variability of at least one lipooligosaccharide glycosyl transferase (lgt) gene(s) non-phase variable thus resulting in a bacterium that produces no detectable OMVs displaying LOS glycan structures comprising an oligosaccharide alpha-chain elongating from Hep I having less than four hexose monosaccharides (2Hex or 3Hex) and b) detoxify the LOS

### Genetic Modification: Rendering Gene(s) Non-Phase Variable

There is provided immunogenic compositions comprise isolated gonococcal OMVs obtained or obtainable from a genetically modified Neisseria gonorrhoeae bacterium said bacterium comprising genetic modification(s) that a) render the phase variability of at least one lipooligosaccharide glycosyl transferase (lgt) gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS. In some embodiments said genetic modification(s) results in a non-phase variable (or fixed) LOS having an alpha-chain extending from Hep I comprising at least four hexose monosaccharides. In an embodiment, said genetic modification(s) that render the phase variability of said at least one lgt gene(s) non-phase variable either a) locks expression ON resulting in said at least one lgt gene(s) being constitutively expressed or, b) locks expression OFF resulting in loss of expression of said at least one lgt gene(s).

By “locks expression ON” it is meant that the genetically modified Neisseria gonorrhoeae bacterium comprises genetic modification(s) that fixes the expression of the at least one lgt gene(s) such that expression of full-length, functional gene product(s) may no longer be switched off by phase variation.

The genetic modification(s) that lock expression of said at least one lgt gene(s) ON may be an insertion of extra copies of said at least one lgt gene(s) wherein said inserted copy (or copies) are constitutively expressed. However, in a preferred embodiment expression of said at least one lgt gene(s) is locked ON by mutating the homopolymeric tract of said at least one lgt gene(s) such that said homopolymer is modified or removed. Modifying the homopolymeric tracts can be performed using homologous recombination between a plasmid construct containing the modified tract and the genomic DNA of the strain to be changed after transformation of the strain with the plasmid.

Phase variable gene expression is mediated by slipped-strain mispairing at homopolymeric tracts (or homopolymeric sequences) within the coding regions of particular genes. Single nucleotide insertions or deletions (INDELs) in these genes due to slipped-strand mispairing events can place these genes in or out of frame resulting in variable production of full-length and functional enzymes. In an embodiment, said genetic modification(s) that render the phase variability of at least one lipooligosaccharide glycosyl transferase (lgt) gene(s) non-phase variable is a genetic modification that prevents slipped strand mispairing within the coding region of said at least one igt gene(s).

An example of such a genetic modification is to change the sequence of the homopolymeric nucleotide tract within the open-reading frame of said at least one igt gene(s) such that codons encoding a specific amino acid (e.g. GGG encoding glycine) is modified to a different codon encoding the same amino acid (e.g. GGA, GGC or GGT encoding glycine). Alternatively, a codon encoding a specific amino acid is modified to a codon encoding a conservative mutation. In this scenario it is preferred that more than one (e.g. 2, 3, 4 or more) codons in the homopolymeric tract are changed, for example to encode the same amino acid.

In some embodiments the homopolymer is modified or removed without altering the coding sequence for said at least one lgt gene(s). In an embodiment, the homopolymer is modified or removed whilst maintaining the open-reading frame in frame. Modification or removal of the homopolymeric tract(s) therefore does not impact the coding sequence for the at least one lgt gene(s) ensuring translation of the functional gene products.

In some embodiments expression is locked ON by reducing the length of the homopolymeric nucleotide tract within the open reading frame of the at least one lgt gene(s) whilst maintaining the open-reading frame in frame. In some embodiments, reducing the length of the homopolymeric nucleotide tract comprises reducing the tract to a smaller number of consecutive nucleotides. In an embodiment, the homopolymeric G or C tract in the open reading frame of said at least one lgt gene(s) is reduced or modified. In an embodiment, the number of homopolymeric nucleotide tracts are reduced. In some embodiments the homopolymeric G or C tract is between 5 and 25 nucleotides (e.g. G₅ to G₂₅ or C₅ to C₂₅), between 10 and 20 nucleotides (e.g. G₁₀ to G₂₀ or C₁₀ to C₂₀) or preferably between 10 and 15 nucleotides (e.g. G₁₀ to G₂₀ or C₁₀ to C₂₀).

In an embodiment, said genetic modification(s) that render the phase variability of said at least one lgt gene(s) non-phase variable either locks expression ON resulting in said at least one lgt gene(s) being constitutively expressed, or locks expression OFF resulting in loss of expression of said at least one lgt gene(s). In an embodiment, expression is locked OFF either i) by deleting said at least one lgt gene(s) or a portion thereof, ii) by insertional inactivation of said at least one lgt gene(s), and/or iii) by inserting stop codons within the open reading frame(s) of said at least one lgt gene(s).

Genetic modification(s) that lock expression OFF results in the permanent downregulation and/or abolishment of gene expression thus preventing the expression of functional gene product from said at least one lgt gene.

In an embodiment, expression is locked OFF by deleting said at least one lgt gene(s). As previously described (see definitions) deleting said at least one lgt gene(s) may be achieved through a number of genetic techniques that have the potential to render a specific gene inoperable or inactive. Gene deletion or gene knockout might include not only deletion of genetic elements but also addition, substitution or modification, such that the gene is inoperable or inactive, i.e. insertion of a genetic sequence may cause mistranslation of the gene, by for example, incorporating an early stop codon, or by causing a missense translation. Genes may for example be deleted by replacement of the gene, or a fragment of said gene, with a different heterologous gene, for example by homologous recombination.

In an embodiment, expression of the at least one lgt gene(s) is locked OFF by insertional inactivation of said least one lgt gene. Insertional inactivation describes the process where recombinant or foreign DNA is inserted into the coding sequence of gene which results in gene inactivation/disablement. The recombinant or foreign DNA may encode, for example, an antibiotic resistance gene enabling selection of insertional inactivation mutants. In this respect insertional inactivation may be considered a gene deletion technique. Thus, in an embodiment, expression is locked OFF by deleting said at least one lgt gene(s) for example through insertional inactivation. In an embodiment, expression of the at least one lgt gene(s) is locked OFF by inserting stop codons within the open reading frame(s) of the least one lgt gene(s).

The lipooligosaccharide glycosyl transferase enzymes are the gene products of the lgt genes. The lgt family modulate the synthesis of the oligosaccharide chains that elongate from the core heptoses of gonococcal LOS. As described herein said at least one lgt gene(s) comprise at least one, more or all of lgtA, lgtC, lgtD and/or lgtG. As such, in an embodiment, there is provided an immunogenic composition comprising isolated gonococcal OMVs obtained or obtainable from a genetically modified Neisseria gonorrhoeae bacterium said bacterium comprising genetic modification(s) that a) render the phase variability of at least one lgt gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS, wherein said at least one lgt gene(s) comprise at least one, more or all of lgtA, lgtC, lgtD and/or lgtG. In an embodiment, said at least one lgt gene(s) is lgtA. In an embodiment, said at least one lgt gene(s) is lgtA and lgtC. In an embodiment, said at least one lgt gene(s) is lgtA, lgtC and lgtD.

In an embodiment, there is provided an immunogenic composition comprising isolated gonococcal OMVs obtained or obtainable from a genetically modified Neisseria gonorrhoeae bacterium said bacterium comprising genetic modification(s) that a) render the phase variability of at least one lgt gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS wherein said genetic modification(s) that render the phase variability of at least one lgt gene(s) non-phase variable renders the phase variability of i) lgtA non-phase variable and locked ON resulting in lgtA being constitutively expressed.

In an embodiment, there is provided an immunogenic composition comprising isolated gonococcal OMVs obtained or obtainable from a genetically modified Neisseria gonorrhoeae bacterium said bacterium comprising genetic modification(s) that a) render the phase variability of at least one lgt gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS wherein said genetic modification(s) that render the phase variability of at least one lgt gene(s) non-phase variable renders the phase variability of i) lgtA non-phase variable and locked ON resulting in lgtA being constitutively expressed and ii) lgtC non-phase variable and locked OFF resulting in loss of expression of lgtC.

Locking the expression of lgtA ON resulting in lgtA being constitutively expressed ensures that the lgtA enzyme, that is responsible for the addition of the fourth hexose monosaccharide (galactose), is constitutively expressed. Locking the expression of lgtC OFF resulting in loss of expression of lgtC ensures that there is no termination of the Hep I oligosaccharide chain (3Hex) via the addition of a galactose (Gal), that can undergo sialylation (Neu5Ac). Expression of lgtC thus creates a shorter chain (Galα1-4Galβ1-4Glcβ1-4-) attached to Hep I (also referred to as P^{K}-like LOS) which needs to be avoided to ensure the genetically modified bacterium produces OMVs displaying LOS glycan structures comprising an oligosaccharide alpha-chain elongating from Hep I having at least four hexose monosaccharides (4Hex or 5Hex)

In an embodiment, said genetic modification(s) that render the phase variability of at least one lgt gene(s) non-phase variable, renders the phase variability of i) lgtA non-phase variable and locked ON resulting in lgtA being constitutively expressed, ii) lgtC non-phase variable and locked OFF resulting in loss of expression of lgtC and iii) lgtD non-phase variable and locked ON resulting in lgtD being constitutively expressed. Locking of lgtA ON and lgtC off ensures that the OMVs display LOS glycan structures comprising an oligosaccharide alpha-chain elongating from Hep I having at least four hexose monosaccharides (4Hex or 5Hex). Locking the expression of lgtD ON resulting in lgtD being constitutively expressed ensures that the enzyme responsible for the addition of the fifth hexose monosaccharide (terminal GalNac) is constitutively expressed. Since the lgtD enzyme is not 100% efficient, the result is a genetically modified gonococcus that produces OMVs displaying LOS glycan structures comprising an oligosaccharide alpha-chain elongating from Hep I having either four or five hexose monosaccharides (4HexG−, 4HexG+, 5HexG−, 5HexG+).

In some embodiments it may also be desirable to render the phase variability of lgtG non-phase variable and locked ON resulting in lgtG being constitutively expressed. Constitutive expression of lgtG ensures that the enzyme responsible for the addition of the beta chain is constitutively expressed. Constitutive expression of lgtG ensures that the enzyme responsible for forming the al-3 link between the Glc and Hep II is constitutively expressed. Thus, the lgtG gene product initiates production of the beta-chain (and thus the 2C7 epitope) with full extension of the beta-chain being under the control of the lgtE gene product which is constitutively expressed and naturally non-phase variable and adds Gal to Hep II linked Glc. However, as described herein the presence of a beta-chain (and thus the 2C7 epitope), whilst not detrimental, was not essential for elicitation of bactericidal antibodies following administration of gonococcal OMV vaccines to a subject.

In an embodiment, said genetic modification(s) that render the phase variability of said at least one lgt gene(s) non-phase variable are made within the locus of each specific lgt gene(s). In an embodiment, a plurality of genetic modification(s) are made to render the phase variability of said at least one lgt gene(s) non-phase variable, each genetic modification being within the locus of each specific lgt gene. Thus, in an embodiment, separate genetic modification(s) are made within the locus of the lgtA gene and the lgtC gene. In an embodiment, separate genetic modification(s) are made within the locus of the lgtA gene, the lgtC gene and the lgtD gene.

In a separate embodiment said genetic modification(s) that render the phase variability of said at least one lgt gene(s) non-phase variable are made via replacement of the operon comprising the lgt gene loci. In an embodiment, a single genetic modification(s) is made to replace the operon comprising the lgt gene loci to render the phase variability of said at least one lgt gene(s) non-phase variable. This genetic modification approach thus simplifies the creation of a genetically modified Neisseria gonorrhoeae bacterium that produces OMVs displaying LOS glycan structures comprising an oligosaccharide alpha-chain elongating from Hep I having at least four hexose monosaccharides (4Hex or 5Hex) because it is no longer required to make separate individual modifications to each lgt gene. In an embodiment, a single genetic modification(s) is made to replace the operon comprising the lgt gene loci to render the phase variability of lgtA non-phase variable and locked ON resulting in lgtA being constitutively expressed and to render the phase variability lgtC non-phase variable and locked OFF resulting in loss of expression of lgtC. In an embodiment, the LgtABCDE operon is replaced. In an embodiment, the operon that is replaced comprises lgtA, lgtB, lgtC, lgtD, lgtE genes. In an embodiment, the entire operon comprising the lgt gene loci is replaced. In an embodiment, a portion of the operon is replaced, said portion comprising at least the lgtA, lgtC and optionally lgtD loci.

### Genetic Modification: Detoxification of LOS

There is provided an immunogenic composition comprising isolated gonococcal OMVs obtained or obtainable from a genetically modified Neisseria gonorrhoeae bacterium said bacterium comprising genetic modification(s) that a) render the phase variability of at least one lipooligosaccharide glycosyl transferase (lgt) gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS. In an embodiment, said genetic modification(s) to detoxify the LOS, decreases or abolishes expression and/or function of the lipid A biosynthesis lauroyl acyltransferase (lpxl1) gene, lpxl1 mRNA and/or lpxl1 polypeptide. In an embodiment, said genetic modification(s) to detoxify the LOS decreases or abolishes expression of the lpxl1 gene, lpxl1 mRNA, and/or lpxl1 polypeptide. In an embodiment, said genetic modification(s) to detoxify the LOS decreases or abolishes expression of the lpxl1 polypeptide.

In the context of the present disclosure, “decreased expression” means that the genetically modified Neisseria gonorrhoeae bacterium expresses less lpxl1 mRNA and/or Lpxl1 protein compared to an unmodified (wild-type) Neisseria gonorrhoeae bacterium or a Neisseria gonorrhoeae bacterium comprising the wild-type lpxl1 gene. Expression may be considered decreased when any reduction in mRNA and/or protein expression is observed compared to an unmodified (wild-type) gonococcus or a gonococcus comprising the wild-type lpxl1 gene. Expression may be considered decreased when a reduction of over 5%, over 10%, over 25%, over 50%, over 60%, over 70%, over 80% over 90% or over 95% in mRNA and/or protein expression is observed compared to the mRNA and/or protein expression, respectively, in an unmodified (wild-type) gonococcus or a gonococcus comprising the wild-type lpxl1 gene. In the context of the present disclosure, “abolished expression” means that no Lpxl1 mRNA and/or protein and no Rmp mRNA and/or protein can be detected in the gonococcal bacterium using the technique used by the skilled person to measure expression.

The level of expression of the lpxl1 gene can be measured using techniques well known to the skilled person, for example using polymerase chain reaction (PCR) based techniques (for example using Q/RT-PCR). The level of expression of the Lpxl1 polypeptide can be measured using techniques well known to the skilled person. For example, the level of expression of the Lpxl1 polypeptide can be measured using western blotting or ELISA.

The lpxl1 gene (also referred to as msbB) encodes the polypeptide Lipid A biosynthesis lauroyl acyltransferase (Lpxl1). Lpxl1 plays a role in lipid A biosynthesis. Neisseria organisms genetically modified to provide for decreased or no detectable functional lpxl1 encoded protein produce OMVs with reduced endotoxicity. This is because the amount of lipid A acylation and the nature of the acylation are major factors that affect LOS toxicity [Makda Fisseha et al. Infection and Immunity June 2005, 73 (7) 4070-4080]. Lpxl1 (polypeptide) may also be referred to as the Lpxl1 enzyme. The exact sequence of the lpxl1 gene may differ across different strains of Neisseria gonorrhoeae as will be understood by the person skilled in the art. However, in an embodiment, the genetic modification(s) to detoxify the LOS decreases or abolishes the expression of the lpxl1 gene wherein the lpxl1 gene comprises a sequence at least 80%, at least 85%, at least 90%, at least 91%, at least 92%, at least 93%, at least 94%, at least 95%, at least 96%, at least 97%, at least 98%, at least 99% or 100% identical to the sequence as set forth in SEQ ID NO: 1.

In an embodiment, the genetic modification(s) to detoxify the LOS decreases or abolishes the expression of the lpxl1 polypeptide wherein the lpxl1 comprises an amino acid sequence at least 80%, at least 85%, at least 90%, at least 91%, at least 92%, at least 93%, at least 94%, at least 95%, at least 96%, at least 97%, at least 98%, at least 99% or 100% identical to the sequence as set forth in SEQ ID NO: 2. In an embodiment, said genetically modified Neisseria gonorrhoeae bacterium expresses less than 20%, less than 10%, less than 5% or less than 1% of the lpxl1 polypeptide compared to the expression of the Lpxl1 polypeptide in an either an unmodified (e.g. wild-type) Neisseria gonorrhoeae bacterium or a Neisseria gonorrhoeae bacterium comprising the wild-type lpxl1 gene. In an embodiment, said genetically modified Neisseria gonorrhoeae bacterium does not express the Lpxl1 polypeptide. In an embodiment, said genetically modified Neisseria gonorrhoeae bacterium does not express the Lpxl1 at a detectable level as measured for example by immunoassay. In an embodiment, the said genetically modified Neisseria gonorrhoeae bacterium does not express the Lpxl1 polypeptide at a detectable level as measured by western blot or ELISA.

In an embodiment, said genetic modification(s) to detoxify the LOS is a deletion of the lpxl1 gene. Any suitable technique can be used to delete the endogenous lpxl1 gene (i.e. to generate a gene knockout). Gene knockouts in gonococci can for example be made by transposon mutagenesis, in vitro genetic engineering to modify genes contained on plasmids or Bacterial Artificial Chromosomes (BACs) and moving the modified construct to the organism of interest, and in vivo homologous recombination. In an embodiment, the genes are knocked out by disabling an endogenous promoter, operon or regulatory element that is essential for transcription or translation of the genes. In an embodiment, the genes are deleted using CRISPR-Cas9 technology. In an embodiment, the endogenous lpxl1 gene is deleted by homologous recombination. During the process of homologous recombination, the endogenous lpxl1 gene is deleted by either adding a different gene into the coding sequence of the lpxl1 gene or by replacing the gene, or a portion thereof, with the different gene (e.g., a heterologous gene, or non-functional gene) by recombination. In an embodiment, the heterologous gene is an antibiotic resistance gene.

### Further Genetic Modification: Reduction Modifiable Protein (Rmp)

There is provided an immunogenic composition comprising isolated gonococcal OMVs obtained or obtainable from a genetically modified Neisseria gonorrhoeae bacterium said bacterium comprising genetic modification(s) that a) render the phase variability of at least one lipooligosaccharide glycosyl transferase (lgt) gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS. In an embodiment, said genetically modified Neisseria gonorrhoeae bacterium comprises a yet further genetic modification, wherein said yet further genetic modification decreases or abolishes expression and/or function of the reduction modifiable protein (rmp) gene, rmp mRNA, and/or rmp polypeptide. In an embodiment, said yet further genetic modification(s) decreases or abolishes expression of the rmp gene, rmp mRNA, and/or rmp polypeptide. In an embodiment, said yet further genetic modification(s) decreases or abolishes expression of the rmp polypeptide. In the context of the present disclosure, “decreased expression” means that the genetically modified Neisseria gonorrhoeae bacterium comprising said yet further genetic modification expresses less rmp mRNA and/or rmp protein compared to an unmodified (wild-type) Neisseria gonorrhoeae bacterium or a Neisseria gonorrhoeae bacterium comprising the wild-type rmp gene. Expression may be considered decreased when any reduction in mRNA and/or protein expression is observed compared to an unmodified (wild-type) gonococcus or a gonococcus comprising the wild-type rmp gene. Expression may be considered decreased when a reduction of over 5%, over 10%, over 25%, over 50%, over 60%, over 70%, over 80% over 90% or over 95% in mRNA and/or protein expression is observed compared to the mRNA and/or protein expression, respectively, in an unmodified (wild-type) gonococcus or a gonococcus comprising the wild-type rmp gene. In the context of the present disclosure, “abolished expression” means that no Rmp mRNA and/or protein can be detected in the gonococcal bacterium using the technique used by the skilled person to measure expression. The level of expression of the rmp genes can be measured using techniques well known to the skilled person, for example using polymerase chain reaction (PCR) based techniques (for example using Q/RT-PCR). The level of expression of the Rmp polypeptide can be measured using SDS-PAGE and LC/MS-MS or via immunoassays such as western blot or ELISA.

The rmp gene encodes the polypeptide reduction modifiable protein (Rmp). Reduction modifiable protein (Rmp), previously known as PIII, has been shown to induce blocking antibodies which could inhibit the effect of other bactericidal antibodies [Gulati S, et al. J Infect Dis. 2015; 212(2):311-315][Joiner K A et al. J Clin Invest. 1985; 76(5):1765-1772]. It may therefore be preferable to yet further genetically modify the Neisseria gonorrhoeae bacterium to decrease or abolish the expression and/or function of the reduction modifiable protein (rmp) gene, rmp mRNA, and/or rmp polypeptide. The exact sequence of the rmp gene may differ across different strains of Neisseria gonorrhoeae as will be understood by the person skilled in the art. However, in some embodiments the yet further genetic modification(s) decreases or abolishes the expression of the rmp gene wherein the rmp gene comprises a sequence at least 80%, at least 85%, at least 90%, at least 91%, at least 92%, at least 93%, at least 94%, at least 95%, at least 96%, at least 97%, at least 98%, at least 99% or 100% identical to the sequence as set forth in SEQ ID NO: 3.

In an embodiment, the yet further genetic modification(s) decreases or abolishes the expression of the rmp polypeptide, wherein the rmp polypeptide comprises an amino acid sequence at least 80%, at least 85%, at least 90%, at least 91%, at least 92%, at least 93%, at least 94%, at least 95%, at least 96%, at least 97%, at least 98%, at least 99% or 100% identical to the sequence as set forth in SEQ ID NO: 4. In an embodiment, said genetically modified Neisseria gonorrhoeae bacterium expresses less than 20%, less than 10%, less than 5% or less than 1% of the rmp polypeptide compared to the expression of the rmp polypeptide in an either an unmodified (e.g. wild-type) Neisseria gonorrhoeae bacterium or a Neisseria gonorrhoeae bacterium comprising the wild-type rmp. In an embodiment, said genetically modified Neisseria gonorrhoeae bacterium does not express the rmp polypeptide. In an embodiment, said genetically modified Neisseria gonorrhoeae bacterium does not express the rmp polypeptide at a detectable level as measured for example by immunoassay.

In an embodiment, said yet further genetic modification(s) is deletion of the rmp gene. As described previously any suitable technique can be used to delete the endogenous rmp gene (i.e. to generate a gene knockout). Preferably deletion of the rmp gene is achieved via homologous recombination.

In another aspect there is provided immunogenic compositions comprising isolated gonococcal OMVs obtained or obtainable from a genetically modified Neisseria gonorrhoeae bacterium. In an embodiment, said bacterium is present as a culture of genetically modified Neisseria gonorrhoeae bacteria. In an embodiment, said culture of genetically modified Neisseria gonorrhoeae is grown on agar medium enriched with between 0.1% and 10% v/v isovitalex, between 0.5% and 5% v/v isovitalex or between 0.75% and 2% v/v isovitalex. Preferably the concentration of isovitalex is 1% v/v. Said agar medium may optionally comprise starch (e.g. corn starch) to absorb toxic metabolites. Said agar medium may optionally further comprise a phosphate buffer to substantially prevent pH alteration. In an embodiment, said culture of genetically modified Neisseria gonorrhoeae is grown in liquid culture. A liquid culture of genetically modified Neisseria gonorrhoeae comprises Neisseria gonorrhoeae that have been transferred to liquid culture following initial growth in a plate.

In a further aspect, there is provided immunogenic compositions comprising isolated gonococcal outer membrane vesicles (OMVs) wherein said composition substantially comprises OMVs that display lipooligosaccharide (LOS) glycan structures comprising an oligosaccharide alpha-chain elongating from Hep I having at least four hexose monosaccharides (4Hex or 5Hex) and wherein said LOS comprises a pentaacylated lipid A rather than a hexaacylated lipid A. Said composition comprises isolated gonococcal OMVs that only display LOS glycan structures comprising an oligosaccharide alpha-chain extending from Hep I having at least four hexose monosaccharides. In an embodiment, said composition comprises no detectable OMVs that display LOS glycan structures comprising an oligosaccharide alpha-chain elongating from Hep I having less than four hexose monosaccharides (2Hex or 3Hex). Thus there is provided an immunogenic compositions comprising isolated gonococcal outer membrane vesicles (OMVs) wherein said composition comprises no detectable OMVs that display lipooligosaccharide (LOS) glycan structures comprising an oligosaccharide alpha-chain elongating from Hep I having less than four hexose monosaccharides (2Hex or 3Hex) and wherein said LOS comprises a pentaacylated lipid A rather than a hexaacylated lipid A. Detection of OMVs displaying shorter alpha-chain structures (i.e. 2Hex or 3Hex) can be performed using a number of techniques that are known to the person skilled in the art.

One such technique to detect whether shorter alpha-chain structures are present is to utilise anti-LOS specific mAbs. A number of the monoclonal antibodies are described in Table 2 below which may be used to determine which LOS structures are displayed. For example, binding of the L1 mAb indicates the presence of 3HexG+ or 3HexG− structures. Binding of the 2C7 mAb indicates the presence of two lactoses, one β-linked to Hep I, the other α-linked to Hep II (thus 2HexG+, 3HexG+, 4HexG+ and 5HexG+).

TABLE 2
Anti-LOS specific mAbs

| mAb | Epitope | Structural Requirements |
|---|---|---|
| 2C7 | Gal-Glc-Hep I | Further Hep I extensions may permit binding |
| | Gal-Glc-Hep II | |
| 17-1-L1 | Gal-Gal-Glc-Hep I | Hep II extension may permit binding |
| (“L1”) | | |
| 4C4 | Glc-Hep I | Hep I extension beyond Gal-Glc-Hep I abrogates the |
| | | binding. Hep II extension may abrogate binding. |
| L3, 7, 9 | Gal-GlcNAc-Gal- | Hep I extension (GalNAc or NeuNAc) abrogates the |
| | Glc-Hep I | binding; Hep II extension abrogates the binding |
| 2-1-L8 | Gal-Glc-Hep I | Hep I extension abrogates the binding |
| (“L8”) | | |
| 3F11 | Gal-GlcNAc-Gal- | Hep I extension (GalNAc or NeuNAc) abrogates the |
| | Glc-Hep I | binding |

In some embodiments absence of mAb L1 and mAb 4C4 binding indicates that there are no detectable OMVs displaying LOS glycan structures comprising an oligosaccharide alpha-chain elongating from Hep I having less than four hexose monosaccharides (2Hex or 3Hex). This is because mAb L1 binds 3Hex LOS structures (3HexG− and 3HexG+) and mAb 4C4 binds 2Hex structures (2HexG− and 2HexG+). In an embodiment, absence of mAb L1 and mAb 4C4 binding is confirmed by a binding immunoassay, optionally a western blot.

It is however possible that the presence of an α-linked lactose to Hep II can abrogate binding of the 4C4 mAb. Thus, absence of 4C4 binding cannot definitively rule out presence of 2HexG+ structures. Therefore, in addition, it may be desirable to analyse a) whether mAb 2C7 binds and b) the molecular weight dependent migration of a 2C7 reactive band via western blot or silver staining. The 2C7 mAb binds to 5HexG+, 4HexG+, 3HexG+ and 2HexG+ LOS structures. The 2HexG+ LOS structure migrates the furthest in silver staining due to its lower molecular weight as compared to the 3HexG+ but most especially the 4HexG+ and 5HexG+ structures.

As such, a more conclusive method to determine if there are detectable OMVs that display LOS glycan structures comprising an oligosaccharide alpha-chain elongating from Hep I having less than four hexose monosaccharides (2Hex or 3Hex) is as follows:
1. Assess binding of the L1 mAb wherein absence of L1 binding confirms absence of 3Hex structures (3HexG+ or 3HexG− structures present)
2. Assess binding of 4C4 mAb wherein absence of 4C4 binding confirms absence 2HexG− structures and is indicative that 2HexG+ structures are absent. Thus, the only remaining assessment required is confirmation of no 2HexG+ structures.
3. Assess binding of 2C7 mAb and height of 2C7 reactive band wherein absence of 2C7 binding confirms no 2HexG+ structures. If 2C7 binding is present assess height of 2C7 reactive band relative to 3.5 kDa marker wherein absence of 2C7 reactive band below the 3.5 kDa is confirmatory that no 2HexG+ structures present.

As such, in an embodiment, absence of a mAb 2C7 reactive LOS band below 3.5 kDa is confirmatory that there is no detectable OMVs displaying LOS glycan structures comprising an oligosaccharide alpha-chain elongating from Hep I having less than four hexose monosaccharides (2Hex or 3Hex). In an embodiment, absence of both mAb L1 and mAb 4C4 binding and absence of a 2C7 reactive LOS band below 3.5 kDa is confirmatory that there is no detectable OMVs displaying LOS glycan structures comprising an oligosaccharide alpha-chain elongating from Hep I having less than four hexose monosaccharides (2Hex or 3Hex).

In a separate embodiment, there is provided an immunogenic composition comprising isolated gonococcal OMVs wherein said composition substantially comprises OMVs that display lipooligosaccharide (LOS) glycan structures comprising an oligosaccharide alpha-chain elongating from Hep I having at least four hexose monosaccharides (4Hex or 5Hex) and wherein said LOS comprises a pentaacylated lipid A rather than a hexaacylated lipid A and wherein over 80%, over 85%, over 90% or over 95% of the OMVs display LOS glycan structures comprising an oligosaccharide alpha-chain elongating from Hep I having at least four hexose monosaccharides (4Hex or 5Hex). A composition that “substantially” comprises OMVs that display said LOS structures is a composition that entirely comprises OMVs that display said LOS glycan structures beyond the background chance that OMVs displaying 2Hex or 3Hex LOS glycan structures are present, for example a trace quantity of OMVs displaying 2Hex or 3Hex LOS glycan structures. In an embodiment, over 97%, over 98% over 99%, over 99.5% or 100% of OMVs display LOS glycan structures comprising an oligosaccharide alpha-chain elongating from Hep I having at least four hexose monosaccharides (4Hex or 5Hex).

### Outer Membrane Vesicles

As disclosed herein, there is provided
an immunogenic composition comprising isolated gonococcal OMVs obtained or obtainable from a genetically modified Neisseria gonorrhoeae bacterium said bacterium comprising genetic modification(s) that a) render the phase variability of at least one lipooligosaccharide glycosyl transferase (lgt) gene(s) non-phase variable thus resulting in a bacterium that produces OMVs displaying LOS glycan structures comprising an oligosaccharide alpha-chain elongating from Hep I having at least four hexose monosaccharides (4Hex or 5Hex) and b) detoxify the LOS.
an immunogenic composition comprising isolated gonococcal outer membrane vesicles (OMVs) wherein said composition substantially comprises OMVs that display lipooligosaccharide (LOS) glycan structures comprising an oligosaccharide alpha-chain elongating from Hep I having at least four hexose monosaccharides (4Hex or 5Hex) and wherein said LOS is detoxified.
Both of said immunogenic compositions comprise isolated gonococcal OMVs. Said isolated gonococcal OMVs display LOS which is detoxified. Such detoxification eliminates the endotoxicity of the LOS thus rendering the OMV safe for administration to a subject (e.g. a human being), for example as a vaccine.

In an embodiment, the isolated gonococcal OMVs comprise LOS that is detoxified. Said detoxification is achieved by a genetic modification (to detoxify the LOS) but may be further achieved via detergent-based extraction techniques. In an embodiment, the LOS is detoxified via more than one method i.e. by both genetic modification and detergent extraction.

In an embodiment, the isolated gonococcal OMVs comprise LOS that is detoxified via detergent extraction. In an embodiment, extraction using a deoxycholate or sodium dodecyl sulfate (SDS) detergent detoxifies and reduces the LOS content in said gonococcal OMVs. In an embodiment, said reduced LOS content is measured in comparison to native gonococcal OMVs (or blebs) that have not been detoxified via detergent extraction.

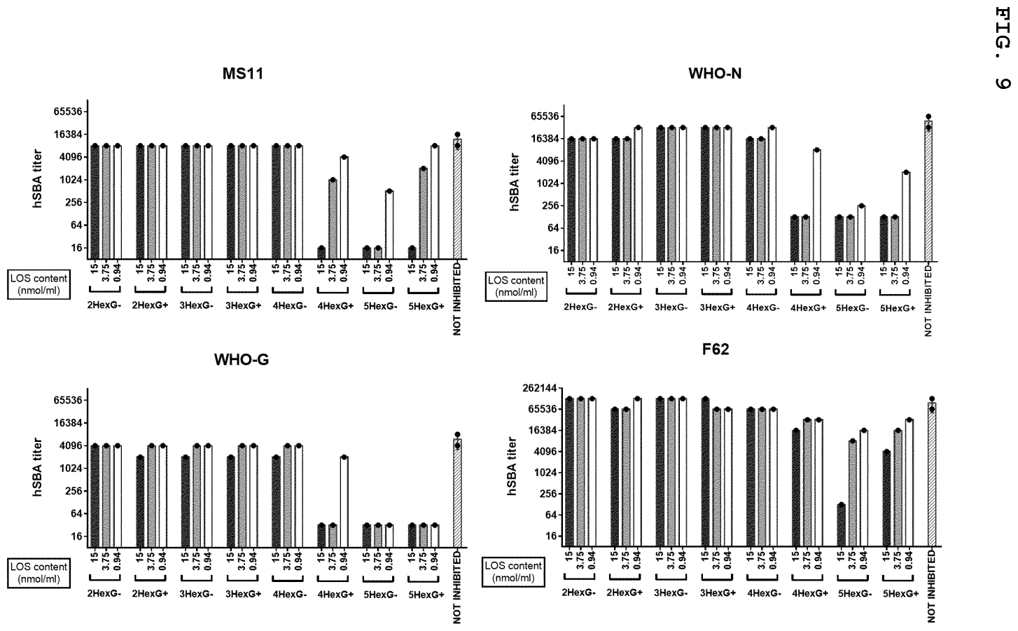

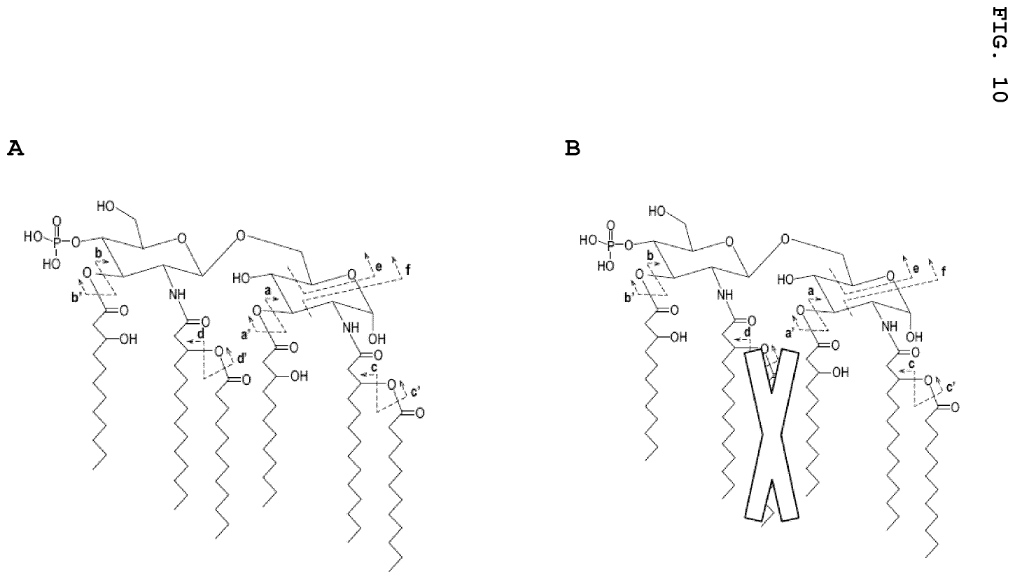

Preferably the LOS is detoxified via genetic modification. Where the LOS is detoxified via genetic modification the isolated gonococcal OMVs comprise detoxified LOS having a pentaacylated lipid A rather than a hexaacylated lipid A. In an embodiment, the isolated gonococcal OMVs comprise reduced levels of hexa-acylated lipid A compared to the levels of hexa-acylated lipid A in a comparator OMV from wherein said comparator OMV is obtained either from a wild-type Neisseria gonorrhoeae bacterium or from a Neisseria gonorrhoeae bacterium that lacks said genetic modification(s) to detoxify the LOS. In an embodiment, the isolated gonococcal OMVs comprise increased levels of penta-acylated lipid A that lacks lauric acid compared to the levels of penta-acylated lipid A that lacks lauric acid from a comparator OMV wherein said comparator OMV is obtained either from a wild-type Neisseria gonorrhoeae bacterium or from a Neisseria gonorrhoeae bacterium that lacks said genetic modification(s) to detoxify the LOS. The levels of hexa/penta-acylated lipid A can be determined for example by analysing the acylation state of lipid A using mass spectrometry (e.g. as described in van der Ley et al. Infection and immunity vol. 69,10 (2001): 5981-90). In an embodiment, said detoxified LOS lacks the secondary lauroyl chain from the non-reducing end of the GlcN disaccharide. The structure of lipid A in its hexa/penta-acylated form is shown in FIG. 10.

The immunogenic compositions comprise isolated gonococcal OMVs that display LOS glycan structures comprising an oligosaccharide alpha-chain elongating from Hep I having at least four hexose monosaccharides (4Hex or 5Hex). In an embodiment, said oligosaccharide alpha-chain elongating from Hep I comprises either β-Gal-(1→4)-β-GlcNAc-(1→3)-β-Gal-(1→4)-β-Glc-(1→4)-Hep I (4Hex) or β-GalNAc-(1→3)-β-Gal-(1→4)-β-GlcNAc-(1→3)-β-Gal-(1→4)-β-Glc-(1→4)-Hep I (5Hex). In an embodiment, said oligosaccharide alpha-chain comprises either a terminal galactose (4Hex) or a terminal GalNac (5Hex). A terminal galactose may also be displayed by LOS comprising a 2Hex alpha-chain or a 3Hex alpha-chain. However as used herein “terminal galactose (4Hex)” does not include the 2Hex or 3Hex structure. In an embodiment, said oligosaccharide alpha-chain comprises either a D-linked terminal galactose (4Hex) or a terminal GalNac (5Hex). A β-linked terminal galactose may also be displayed by LOS comprising a 2Hex alpha-chain. However as used herein “β-linked terminal galactose (4Hex)” does not include the 2Hex structure.

In an embodiment, the immunogenic compositions comprise isolated gonococcal OMVs that display LOS glycan structures comprising an oligosaccharide alpha-chain elongating from Hep I having at least four hexose monosaccharides (4Hex or 5Hex) and wherein said LOS glycan structures comprise Hep II. In an embodiment, said LOS glycan structures comprise Hep II and said Hep II either comprises its beta-oligosaccharide chain (4HexG+ or 5HexG+) or does not comprise its beta-oligosaccharide chain (4HexG− or 5HexG−). In an embodiment, said beta-oligosaccharide chain is a disaccharide chain. In an embodiment, said beta-oligosaccharide chain is a Hep II-linked lactose. In an embodiment, said beta-oligosaccharide chain comprises β-Gal-(1→4)-α-Glc-(1→3)-Hep II.

In an embodiment, the immunogenic compositions comprise isolated gonococcal OMVs that display LOS glucan structures having an oligosaccharide alpha-chain elongating from Hep I having at least four hexose monosaccharides (4Hex or 5Hex) wherein said LOS glycan structures comprise Hep II and wherein Hep II does not comprise its beta oligosaccharide chain (4HexG− or 5HexG−). In an embodiment, Hep II does not comprise its beta oligosaccharide chain, said beta oligosaccharide chain comprising β-Gal-(1→4)-α-Glc-(1→3)-Hep II. In an embodiment, said Hep II does not comprise a Hep II-linked lactose. Thus, in an embodiment, said immunogenic compositions comprise isolated gonococcal OMVs that display 4HexG− or 5HexG− LOS glycan structures.

In an embodiment, the immunogenic compositions comprise isolated gonococcal OMVs wherein the 2C7 epitope is either present or absent. In an embodiment, the immunogenic compositions comprise isolated gonococcal OMVs that display LOS glycan structures wherein said LOS glycan structures either comprise the 2C7 epitope or the 2C7 epitope is absent.

In an embodiment, the immunogenic compositions comprise isolated gonococcal OMVs that display LOS glycan structures wherein said LOS glycan structures comprise the 2C7 epitope (4HexG+ or 5HexG+) i.e., the 2C7 epitope is present. In an embodiment, the immunogenic compositions comprise isolated gonococcal OMVs that display LOS glycan structures, wherein the 2C7 mAb is capable of binding to said LOS glycan structures. In an embodiment, the 2C7 mAb comprises a variable heavy (VH) domain, a variable light (VL) domain wherein the VH domain comprises a sequence at least 80%, at least 85%, at least 90%, at least 95% or 100% identical to the amino acid sequence set forth in SEQ ID NO: 5 and wherein the VL domain comprises a sequence at least 80%, at least 85%, at least 90%, at least 95% or 100% identical to the amino acid sequence set forth in SEQ ID NO: 6. In an embodiment, the 2C7 mAb comprises (i) any one or a combination of complementarity determining regions (CDRs) selected from CDRH1, CDRH2 or CDRH3 from SEQ ID NO: 5 and/or CDRL1, CDRL2, CDRL3 from SEQ ID NO: 6 or (ii) a CDR variant of (i) wherein the variant has 1, 2 or 3 amino acid modification(s) in each CDR. In an embodiment, the 2C7 mAb comprises any one or a combination of all of the following CDRs: (a) CDRH1 of SEQ ID NO: 7, (b) CDRH2 of SEQ ID NO: 8, (c) CDRH3 of SEQ ID NO: 9, (d) CDRL1 of SEQ ID NO: 10, (e) CDRL2 of SEQ ID NO: 11 and/or (f) CDRL3 of SEQ ID NO: 12. In an embodiment, the 2C7 mAb is a mouse monoclonal antibody. In an embodiment, the 2C7 mAb may comprise a humanized VH domain or a humanized heavy chain sequence and/or a humanized VL domain or a humanized light chain sequence, which comprise the CDRs as described above. As used herein VH domain and VL domain refers to the variable portions of the heavy (VH) or light (VL) chain respectively. These domains form the binding pocket which binds the specific antigens and contains the major diversity of the immunoglobulin.

The 2C7 epitope comprises two lactoses, one β-linked to Hep I, the other α-linked to Hep II. Thus, the 2C7 epitope is present when the isolated gonococcal OMVs comprise 2HexG+, 3HexG+, 4HexG+ or 5HexG+ LOS glycan structures. Extension of the Hep I linked alpha-chain does not abrogate mAb 2C7 binding, i.e. the 2C7 epitope is present despite extension of the alpha-chain beyond the minimal 2C7 epitope structure or the minimal structure for 2C7 mAb binding (2HexG+).

In an embodiment, the immunogenic compositions comprise isolated gonococcal OMVs that display LOS glycan structures wherein said LOS glycan structures do not comprise the 2C7 epitope (i.e. the 2C7 epitope is absent). In an embodiment, the immunogenic compositions comprise isolated gonococcal OMVs that display LOS glycan structures, wherein the 2C7 mAb is incapable of binding to said LOS glycan structures. In an embodiment, the immunogenic compositions comprise isolated gonococcal OMVs that display LOS glycan structures having an oligosaccharide alpha-chain elongating from Hep I having at least four hexose monosaccharides (4Hex or 5Hex) wherein said LOS glycan structures do not comprise the 2C7 epitope (4HexG− or 5HexG−).

In an embodiment, the immunogenic compositions comprise isolated gonococcal OMVs wherein said gonococcal OMVs comprise reduced levels or no detectable level of rmp polypeptide in comparison to an OMV from a wild-type Neisseria gonorrhoeae. In an embodiment, said gonococcal OMVs comprise reduced levels or no detectable level of rmp polypeptide in comparison to an OMV from Neisseria gonorrhoeae comprising the wild-type rmp gene. In an embodiment, reduced levels or no detectable level of rmp polypeptide is measured by immunoassay (for example by western blot or ELISA assay). In an embodiment, said reduced levels or no detectable level of rmp polypeptide is the result of a genetic modification, said genetic modification being to decrease or abolish the expression and/or function of the rmp gene, rmp mRNA, and/or rmp polypeptide. In an embodiment, said reduced levels or no detectable level of rmp polypeptide is the result of deletion of the rmp gene. In an embodiment, the outer membrane vesicles do not comprise rmp. In an embodiment, the outer membrane vesicles do not express rmp on the surface of said OMVs.

In an embodiment, the immunogenic compositions comprise isolated gonococcal OMVs wherein said isolated gonococcal OMVs are isolated and purified. The purification preferably involves separating the OMVs from living and/or intact N. gonorrhoea bacteria e.g. by using low speed centrifugation to pellet cells while leaving vesicles in suspension and/or by size-based filtration using a filter, such as a 0.22 μm filter, which allows the blebs to pass through but which does not allow intact bacteria to pass through. Thus, unlike the culture medium, OMV containing immunogenic compositions will generally be substantially free from whole bacteria, whether living or dead. The size of the vesicles means that they can readily be separated from whole bacteria by filtration e.g. as typically used for filter sterilisation. Although blebs will pass through a standard 0.22 μm filters, these can rapidly become clogged by other material, and so it may be useful to perform sequential steps of filter sterilisation through a series of filters of decreasing pore size before using a 0.22 μm filter. Examples of preceding filters would be those with pore size of 0.8 μm, 0.45 μm, etc. In an embodiment, the outer membrane vesicles are purified via filtering through a sterile filter with a pore size of less than 0.5, 0.4 or 0.3 μm. In an embodiment, said isolated gonococcal OMVs are purified by tangential flow filtration.

In an embodiment, the immunogenic composition comprises isolated gonococcal OMVs wherein said isolated gonococcal OMVs are native OMVs (nOMVs). In a preferred embodiment, the outer membrane vesicles are obtained via non-detergent extraction. The outer membrane vesicles are obtained from blebbing or is obtained from disruption of the outer membrane, wherein said disruption does not substantially comprise detergent extraction of the OMV from the outer membrane. Preferred methods for obtaining outer membranes vesicles are therefore performed substantially in the absence of detergent using techniques such as sonication, homogenization, microfluidisation, cavitation, osmotic shock, grinding, French press, blending, etc. Methods using no or low detergent can retain useful antigens as described in [WO2004/019977].

In an embodiment, the immunogenic compositions further comprise a pharmaceutically acceptable excipient. Suitable excipients may for example include sodium salts (e.g. sodium chloride) to provide tonicity. Other salts that may be present include potassium chloride, potassium dihydrogen phosphate, disodium phosphate dehydrate, magnesium chloride, calcium chloride etc. Immunogenic compositions may further comprise detergent e.g. a Tween (polysorbate). Immunogenic compositions may include one or more buffers. Typical buffers include: a phosphate buffer; a Tris buffer; a borate buffer; a succinate buffer; a histidine buffer or a citrate buffer.

The immunogenic compositions may be prepared in various forms. Compositions will generally be administered to a subject (e.g. a mammal) in aqueous form however, prior to administration, the composition may have been in a non-aqueous form (e.g. dried or lyophilized). The compositions may be prepared in liquid form as injectables (either as solutions or suspensions). Immunogenic compositions may include a preservative for example thiomersal and/or 2-phenoxyethanol. It is preferred however that the composition be substantially free form mercurial material. Vaccines containing no mercury are more preferred.

In an embodiment, the immunogenic compositions further comprise an adjuvant. The immunogenic compositions may further comprise an adjuvant such that, when administered to a subject in conjunction with said isolated gonococcal OMVs, an increased or enhanced immune response to the antigen or antigens present on the surface of the OMVs is observed. The immunogenic compositions may further comprise an adjuvant such that, when administered to a subject in conjunction with isolated gonococcal OMVs displaying LOS glycan structures comprising an oligosaccharide alpha-chain elongating from Hep I having at least four hexose monosaccharides (4Hex or 5Hex), an increased or enhanced immune response to said LOS glycan structures is observed. Said increased or enhanced immune response is measured in comparison to a non-adjuvanted vaccine composition (for example a vaccine composition comprising isolated gonococcal OMVs displaying LOS glycan structures comprising an oligosaccharide alpha-chain elongating from Hep I having at least four hexose monosaccharides (4Hex or 5Hex) wherein said vaccine composition is non-adjuvanted). The immunogenic compositions may further comprise an adjuvant such that, when administered to a subject in conjunction with said isolated gonococcal OMVs, reduced reactogenicity is observed or reactogenicity is minimised or abolished.

In an embodiment, the immunogenic compositions further comprise an adjuvant wherein the adjuvant is an aluminium salt adjuvant. Suitable aluminium salt adjuvants include hydroxides, phosphates or mixtures thereof. The salts can take any suitable form (e.g. gel, crystalline, amorphous etc) with adsorption of the antigen to the salt being preferred. In an embodiment, the aluminium salt adjuvant is aluminium hydroxide or aluminium phosphate. In an embodiment, the adjuvant is aluminium hydroxide. In an embodiment, the adjuvant is aluminium phosphate. In an embodiment, the OMVs are adsorbed onto aluminium hydroxide or aluminium phosphate. In an embodiment, the adjuvant is not gel-based.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ [Chapter 9 of Vaccine Design: The Subunit and Adjuvant Approach (eds. Powell & Newman) Plenum Press 1995]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

In an embodiment, the immunogenic compositions elicit bactericidal antibodies following administration to a subject. In an embodiment, following administration of the immunogenic composition to a subject, said immunogenic composition induce antibodies that are bactericidal against both homologous and heterologous strain(s) of Neisseria gonorrhoeae. Induction of antibodies that are bactericidal is typically measured via the serum bactericidal activity (SBA) assay which measures bacterial killing mediated by complement (an exemplar method to measure SBA can be found in Example 1 herein). As used herein, the term “heterologous strain(s)” refers to strain(s) of Neisseria gonorrhoeae which are different from the Neisseria gonorrhoeae strain from which the OMVs used to immunize the subject was derived. For example, if the immunogenic composition comprises isolated gonococcal OMVs obtained or obtainable from a genetically modified Neisseria gonorrhoeae bacterium of strain F62, any other gonococcal strain (i.e. other than F62) falls within the meaning of the term “heterologous strain(s)”. Thus, in an embodiment, heterologous strain(s) of Neisseria gonorrhoeae comprise strain(s) of Neisseria gonorrhoeae except for the strain of Neisseria gonorrhoeae from which the composition of isolated gonococcal OMVs were obtained or obtainable from.

As used herein, the term “homologous strain(s)” refers to the same strain(s) of Neisseria gonorrhoeae from which the OMVs used to immunize the subject was derived. For example, if the immunogenic composition comprises isolated gonococcal OMVs obtained or obtainable from a genetically modified Neisseria gonorrhoeae bacterium of strain F62, then the F62 strain would be referred to as the “homologous strain”. In an embodiment, the immunogenic compositions are capable of eliciting cross-bactericidal antibody titres.

In an embodiment, the immunogenic composition, following administration to a subject, elicits antibodies that are capable of inhibiting the adhesion of Neisseria gonorrhoeae to epithelial cells, optionally urethral epithelial cells. In an embodiment, the immunogenic composition, following administration to a subject, elicits antibodies that inhibit gonococcal adherence to epithelial cells, optionally urethral epithelial cells. Tests to monitor whether antibodies are capable of inhibiting adhesion of Neisseria gonorrhoeae to epithelial cells include, for example the bacterial adhesion inhibition (BAI) assay, for example as described in Example 1 herein.

### Vaccines and Treatment

In a further aspect there is provided a vaccine comprising the immunogenic compositions. Vaccines may either be prophylactic (i.e. prevent infection) or therapeutic (i.e. to treat infection) but will typically be prophylactic. The vaccines comprise an immunologically effective amount of antigens, wherein said antigens are present on the surface of the OMVs.

Immunogenic compositions and vaccines are provided for use as medicaments. There is thus provided the use isolated gonococcal OMVs as medicaments in the format of the immunogenic compositions and vaccines. Thus, in a further aspect there is provided the immunogenic compositions or vaccine for use in medicine.

In a further aspect there is provided the immunogenic compositions or vaccine for use in the prevention or treatment of Neisseria gonorrhoeae infection or disease. In an embodiment, there is provided the immunogenic composition or vaccine for use in the prevention or treatment of gonorrhea. In an embodiment, the immunogenic composition or vaccine is used in the prevention or treatment of Neisseria gonorrhoeae infection or disease at the urogenital, anorectal and/or oropharyngeal site. In a further embodiment, the immunogenic composition or vaccine is used in the prevention or treatment of gonococcal associated pelvic inflammatory disease, disseminated gonococcal infection, ectopic pregnancy and/or infertility. Preferably the immunogenic compositions or vaccine is for use in the prevention of Neisseria gonorrhoeae infection or disease.

In a further aspect there is provided the immunogenic compositions or vaccine, for use in immunizing a subject against Neisseria gonorrhoeae infection. In a further aspect there is provided the immunogenic compositions or vaccine for use in generating a bactericidal immune response. As such there is provided the use of the immunogenic composition for generating, following administration to a subject, an immune response in said subject wherein said immune response comprises induction of bactericidal antibodies. Thus, there is provided the immunogenic compositions or vaccine for use in inducing antibodies that are bactericidal against N. gonorrhoeae.

In a further aspect there is provided a method for the treatment or prevention of disease caused by N. gonorrhoeae in a subject in need thereof, said method comprising administering to said subject a therapeutically effective amount of the immunogenic compositions or vaccine.

In a further aspect there is provided a method for immunizing a subject in need thereof against N. gonorrhoeae, comprising administering an immunologically effective amount of the immunogenic compositions or vaccine.

In a further aspect there is provided a method for raising an immune response in a subject, comprising administering the immunogenic compositions or vaccine to a subject.

In a yet further aspect, there is provided the use of the immunogenic compositions or vaccines in the manufacture of a medicament for the treatment or prevention of disease caused by Neisseria gonorrhoeae.

Dosage can be a single dose schedule or a multiple dose schedule. In a further aspect there is provided the immunogenic composition or vaccine for use, the method or the use of wherein at least 2 doses of the composition are administered to a subject. Multiple doses may be used in a primary immunisation schedule and/or in a booster immunisation schedule. A primary dose schedule may be followed by a booster dose schedule. Therefore, in a further embodiment there is provided the immunogenic composition or vaccine for use, the method or the use wherein at least 2 doses of the composition are administered to a subject, wherein at least one dose is a booster dose.

In a further aspect there is provided the immunogenic compositions or vaccine for use, the method or the use wherein the subject is at increased risk of infection with N. gonorrhoea relative to the average risk in the general population. Examples of subjects that are at an increased risk of infection with N. gonorrhoea infection relative to the average risk in the general population might include, but is not limited to, sex workers, men who have sex with men (MSM), pre-exposure prophylaxis (PreP) users, individuals with current or past STI diagnosis, HIV+ individuals who are engaged in care and individuals who are seeking or have sought STI screening or other STI services at a healthcare centre.

In a further aspect there is provided the immunogenic compositions or vaccine for use, method or the use wherein the subject is co-immunised against one or more further infectious agents. Co-immunisation may include immunisation against one or more further infectious agents within the same vaccine (i.e. wherein the vaccine further comprises antigens against one or more further infectious agents). Co-immunisation may however also include immunisation against one or more further infectious agents wherein further vaccines are administered at substantially the same time as the vaccine (for example at the same clinical appointment). For example, the immunogenic composition or vaccine may be administered to a subject alongside a further immunogenic composition or vaccine which comprises antigens against one or more further infectious agents. In an embodiment, the one or more further infectious agents are infectious agents that cause sexually transmitted infections.

Embodiments are further described in the subsequent numbered paragraphs:
1. An immunogenic composition comprising a population of isolated gonococcal outer membrane vesicles (OMVs) said isolated gonococcal OMVs displaying lipooligosaccharide (LOS) glycan structures comprising oligosaccharide alpha-chains elongating from Hep I having at least four hexose monosaccharides (4Hex or 5Hex), wherein the isolated gonococcal OMVs are obtained or obtainable from a genetically modified Neisseria gonorrhoeae strain comprising genetic modifications that
   a) renders the phase variability of at least one LOS glycosyl transferase (lgt) gene(s) non-phase variable, and
   b) detoxify the LOS
   wherein the population of isolated gonococcal OMVs comprise a higher content of LOS glycan structures comprising at least four hexose monosaccharides (4Hex or 5Hex) than that of a comparator population of isolated gonococcal OMVs, the comparator population of isolated gonococcal OMVs being obtained or obtainable from a Neisseria gonorrhoeae bacterium that comprises phase variable lgt genes.
2. The immunogenic composition of paragraph 1 wherein the comparator population of isolated gonococcal OMVs comprises OMVs displaying LOS glycan structures comprising oligosaccharide alpha-chains elongating from Hep I having less than four hexose monosaccharides (2Hex or 3Hex).
3. The immunogenic composition of paragraph 1 or paragraph 2 wherein the population of isolated gonococcal OMVs do not comprise OMVs displaying LOS glycan structures comprising oligosaccharide alpha-chain elongating from Hep I having less than four hexose monosaccharides
4. The immunogenic composition of paragraphs 1-3 wherein the population of isolated gonococcal OMVs do not comprise detectable levels of LOS glycan structures comprising oligosaccharide alpha-chain elongating from Hep I having less than four hexose monosaccharides.
5. An immunogenic composition comprising isolated gonococcal outer membrane vesicles (OMVs) obtained or obtainable from a genetically modified Neisseria gonorrhoeae bacterium said bacterium comprising genetic modification(s) that
   a) render the phase variability of at least one lipooligosaccharide glycosyl transferase (lgt) gene(s) non-phase variable thus resulting in a bacterium that produces OMVs displaying lipooligosaccharide (LOS) glycan structures comprising an oligosaccharide alpha-chain elongating from Hep I having at least four hexose monosaccharides (4Hex or 5Hex) and,
   b) detoxify the LOS
6. The immunogenic composition of paragraph 1 wherein said genetic modification(s) that render the phase variability of at least one lgt gene(s) non-phase variable results in a bacterium that is incapable of producing OMVs displaying LOS glycan structures comprising an oligosaccharide alpha-chain elongating from Hep I having less than four hexose monosaccharides (2Hex or 3Hex)
7. The immunogenic composition of paragraph 5 or paragraph 6 wherein said genetic modification(s) that render the phase variability of said at least one lgt gene(s) non-phase variable either:
   a. locks expression ON resulting in said at least one lgt genes(s) being constitutively expressed or,
   b. locks expression OFF resulting in loss of expression of said at least one lgt gene(s).
8. The immunogenic composition of paragraph 7 wherein said genetic modification(s) that render the phase variability of said at least one lgt gene(s) non-phase variable locks expression ON resulting in said at least one lgt gene(s) being constitutively expressed.
9. The immunogenic composition of paragraph 7 or paragraph 8 wherein expression is locked ON by mutating the homopolymeric tract of said at least one lgt gene(s) such that said homopolymer is modified or removed.
10. The immunogenic composition of paragraph 9 wherein said homopolymer is modified or removed without altering the coding sequence for said at least one lgt gene(s).
11. The immunogenic composition of paragraphs 7-10 wherein expression is locked ON by reducing the length of or modifying the homopolymeric tract within the open reading frame of the at least one lgt gene(s) whilst maintaining the open-reading frame in frame.
12. The immunogenic composition of paragraph 11 wherein the homopolymeric G or C tract in the open reading frame of said at least one lgt gene(s) is reduced or modified
13. The immunogenic composition of paragraph 7 wherein said genetic modification(s) that render the phase variability of said at least one lgt gene(s) non-phase variable locks expression OFF resulting in loss of expression of said at least one lgt gene(s).
14. The immunogenic composition of paragraph 13 wherein expression is locked OFF either:
   i) by deleting said at least one lgt gene(s) or a portion thereof,
   ii) by insertional inactivation of said at least one lgt gene(s), and/or
   iii) by inserting stop codons within the open reading frame of said at least one lgt gene(s).
15. The immunogenic composition of any of paragraphs 5-14 wherein said at least one lgt gene(s) comprise one, more or all of lgtA, lgtC, lgtD and/or lgtG.
16. The immunogenic composition of paragraphs 5-15 wherein said genetic modification(s) that render the phase variability of at least one lgt gene(s) non-phase variable, renders the phase variability of:
   i) lgtA non-phase variable and locked ON resulting in lgtA being constitutively expressed.
17. The immunogenic composition of paragraphs 5-16 wherein said genetic modification(s) that render the phase variability of at least one lgt gene(s) non-phase variable, renders the phase variability of:
   (i) lgtA non-phase variable and locked ON resulting in lgtA being constitutively expressed, and
   (ii) lgtC non-phase variable and locked OFF resulting in loss of expression of lgtC.
18. The immunogenic composition of paragraphs 5-17 wherein said genetic modification(s) that render the phase variability of at least one lgt gene(s) non-phase variable, renders the phase variability of:
   (i) lgtA non-phase variable and locked ON resulting in lgtA being constitutively expressed,
   (ii) lgtC non-phase variable and locked OFF resulting in loss of expression of lgtC, and
   (iii) lgtD non-phase variable and locked ON resulting in lgtD being constitutively expressed.
19. The immunogenic composition of any of paragraphs 5-18 wherein said genetic modification(s) that render the phase variability of said at least one lgt gene(s) non-phase variable are made within the locus of each specific lgt gene(s).
20. The immunogenic composition of any of paragraphs 5-18 wherein said genetic modification(s) that render the phase variability of said at least one lgt gene(s) non-phase variable are made via replacement of the operon comprising the lgt gene loci.
21. The immunogenic composition of paragraphs 5-20 wherein said genetic modification(s) to detoxify the LOS decreases or abolishes expression and/or function of the lipid A biosynthesis lauroyl acyltransferase (lpxl1) gene, lpxl1 mRNA and/or lpxl1 polypeptide.
22. The immunogenic composition of paragraphs 5-21 said genetic modification(s) to detoxify the LOS being deletion of the lpxl1 gene.
23. The immunogenic composition of any preceding paragraph wherein said genetically modified Neisseria gonorrhoeae bacterium comprises a yet further genetic modification, wherein said yet further genetic modification decreases or abolishes expression and/or function of the reduction modifiable protein (rmp) gene, rmp mRNA, and/or rmp polypeptide.
24. The immunogenic composition of paragraph 23 wherein said yet further genetic modification(s) is deletion of the rmp gene
25. The immunogenic composition of any preceding paragraph wherein said genetically modified Neisseria gonorrhoeae bacterium is present as a culture of genetically modified Neisseria gonorrhoeae bacteria.
26. The immunogenic composition of any preceding paragraph wherein the genetically modified Neisseria gonorrhoeae bacterium is derived from strain MS11, BG27, BG8, F62, FA1090, WHO-N, WHO-G or GC_0817560
27. An immunogenic composition comprising isolated gonococcal outer membrane vesicles (OMVs) wherein said composition substantially comprises OMVs that display lipooligosaccharide (LOS) glycan structures comprising an oligosaccharide alpha-chain elongating from Hep I having at least four hexose monosaccharides (4Hex or 5Hex) and wherein said LOS comprises a pentaacylated lipid A rather than a hexaacylated lipid A.
28. The immunogenic composition of paragraphs 5-26 or paragraph 27 said composition comprising no detectable OMVs that display LOS glycan structures comprising an oligosaccharide alpha-chain elongating from Hep I having less than four hexose monosaccharides (2Hex or 3Hex).
29. The immunogenic composition of paragraph 28 wherein absence of mAb L1 and mAb 4C4 binding indicates that there are no detectable OMVs displaying LOS glycan structures comprising an oligosaccharide alpha-chain elongating from Hep I having less than four hexose monosaccharides (2Hex or 3Hex).
30. The immunogenic composition of paragraph 29 wherein absence of mAb L1 and mAb 4C4 binding is confirmed by a binding immunoassay, optionally a western blot.
31. The immunogenic composition of paragraph 28-30 wherein absence of a mAb 2C7 reactive silver stained LOS band below 3.5 kDa is confirmatory that there is no detectable OMVs displaying LOS glycan structures comprising an oligosaccharide alpha-chain elongating from Hep I having less than four hexose monosaccharides (2Hex or 3Hex).
32. The immunogenic composition of paragraphs 28-31 wherein absence of both mAb L1 and mAb 4C4 binding and absence of a 2C7 reactive silver stained LOS band below 3.5 kDa is confirmatory that there is no detectable OMVs displaying LOS glycan structures comprising an oligosaccharide alpha-chain elongating from Hep I having less than four hexose monosaccharides (2Hex or 3Hex).
33. The immunogenic composition of any preceding paragraph wherein over 80%, over 85%, over 90% or over 95% of the isolated gonococcal OMVs display LOS glycan structures comprising an oligosaccharide alpha-chain elongating from Hep I having at least four hexose monosaccharides (4Hex or 5Hex).
34. The immunogenic composition of paragraphs 27-33 wherein said LOS is detoxified.
35. The immunogenic composition of paragraph 34 wherein said detoxified LOS lacks the secondary lauroyl chain from the non-reducing end of the GlcN disaccharide.
36. The immunogenic composition of any preceding paragraph wherein said oligosaccharide alpha-chain elongating from Hep I comprises either β-Gal-(1→4)-β-GlcNAc-(1→3)-β-Gal-(1→4)-β-Glc-(1→4)-Hep I (4Hex) or β-GalNAc-(1→3)-β-Gal-(1→4)-β-GlcNAc-(1→3)-β-Gal-(1→4)-β-Glc-(1→4)-Hep I (5Hex).
37. The immunogenic composition of any preceding paragraph wherein said oligosaccharide alpha-chain comprises either a terminal galactose (4Hex) or a terminal GalNac (5Hex).
38. The immunogenic composition of any preceding paragraph wherein said LOS glycan structures comprise Hep II and said Hep II either:
   a) comprises its beta-oligosaccharide chain β-Gal-(1→4)-α-Glc-(1→3)-Hep II (4HexG+ or 5HexG+) or
   b) does not comprise its beta-oligosaccharide chain (4HexG− or 5HexG−).
39. The immunogenic composition of paragraph 38 wherein Hep II does not comprise its beta oligosaccharide chain (4HexG− or 5HexG−).
40. The immunogenic composition of any preceding paragraph wherein said LOS glycan structures either comprise the 2C7 epitope or the 2C7 epitope is absent.
41. The immunogenic composition of paragraph 40 wherein the 2C7 epitope is absent.
42. The immunogenic composition of any preceding paragraph wherein said gonococcal OMVs comprise reduced levels or no detectable level of rmp polypeptide in comparison to an OMV from a wild-type Neisseria gonorrhoeae.
43. The immunogenic composition of paragraph 42 wherein said isolated gonococcal OMVs do not comprise the reduction modifiable protein (rmp) polypeptide.
44. The immunogenic composition of any preceding paragraph wherein said isolated gonococcal OMVs are isolated and purified.
45. The immunogenic composition of any preceding paragraph wherein said isolated gonococcal OMVs are native OMVs (nOMVs).
46. The immunogenic composition of any preceding paragraph further comprising a pharmaceutically acceptable excipient.
47. The immunogenic composition of any preceding paragraph further comprising an adjuvant.
48. The immunogenic composition of paragraph 47 wherein the adjuvant is an aluminum salt adjuvant
49. The immunogenic composition of paragraph 48 wherein the aluminum salt adjuvant is aluminum hydroxide or aluminum phosphate.
50. The immunogenic composition of any preceding paragraph wherein said immunogenic composition elicits bactericidal antibodies following administration to a subject.
51. The immunogenic composition of any preceding paragraph wherein following administration to a subject, said immunogenic composition elicits antibodies that are bactericidal against both homologous and heterologous strain(s) of Neisseria gonorrhoeae.
52. The immunogenic composition of paragraph 51 wherein heterologous strain(s) of Neisseria gonorrhoeae comprises strain(s) of Neisseria gonorrhoeae except for the strain of Neisseria gonorrhoeae from which the composition of isolated gonococcal OMVs according to paragraphs 1-51 were obtained or obtainable from.
53. A vaccine comprising the immunogenic composition of any of paragraphs 1-52.
54. The immunogenic composition of paragraphs 1-52 or the vaccine of paragraph 53 for use in medicine.
55. The immunogenic composition according to any of paragraphs 1-42 or the vaccine of paragraph 53 for use in the prevention or treatment of Neisseria gonorrhoeae infection or disease.
56. The immunogenic composition according to any of paragraphs 1-52 or the vaccine of paragraph 53, for use in immunizing a subject against N. gonorrhoeae infection.
57. The immunogenic composition according to any of paragraphs 1-52 or the vaccine of paragraph 53 for use in generating a bactericidal immune response.
58. The immunogenic composition according to any of paragraphs 1-52 or the vaccine of paragraph 53, for use in inducing antibodies that are bactericidal against N. gonorrhoeae
59. A method for the treatment or prevention of disease caused by N. gonorrhoeae in a subject in need thereof, said method comprising administering to said subject a therapeutically effective amount of the immunogenic composition according to any of paragraphs 1-52 or the vaccine of paragraph 53.
60. A method for immunizing a subject in need thereof against N. gonorrhoeae, comprising administering an immunologically effective amount of the immunogenic composition according to any of paragraphs 1-52 or vaccine according to paragraph 53 to the subject.
61. A method for raising an immune response in a subject, comprising administering the immunogenic composition according to any of paragraphs 1-52 or the vaccine according to paragraph 53 to a subject.
62. The use of the immunogenic composition according to any of paragraphs 1-52 or the vaccine of paragraph 53 in the manufacture of a medicament for the treatment or prevention of disease caused by Neisseria gonorrhoeae.
63. The immunogenic composition or vaccine for use, the method or the use according to any of paragraphs 54-62, wherein at least 2 doses of the composition are administered to a subject.
64. The immunogenic composition or vaccine for use, the method or the use according to any of paragraphs 54-62 wherein the subject is at increased risk of infection with N. gonorrhoeae relative to the average risk in the general population.
65. The immunogenic composition or vaccine for use, the method or the use according to any of paragraphs 54-62 wherein the subject is co-immunized against one or more further infectious agents.

The invention is further illustrated by the following non-limiting examples.

### EXAMPLES

### Example 1: Materials and Methods

### Bacterial Strains and Growth Conditions

Neisseria gonorrhoeae MS11 Lgt mutant strains: Neisseria gonorrhoeae MS11 Lgt mutant strains used in this work were obtained from Prof. Sanjay Ram (Division of Infectious Diseases and Immunology, University of Massachusetts Medical School). The MS11 lgt mutants were created in the background of N. gonorrhoeae MS11 4/3/1, a variant of MS11 VD300 with an isopropyl-D-thiogalactopyranoside (IPTG)-inducible pilE that controls pilus expression. In these mutant strains the expression of the four phase-variable lgt genes (lgtG, lgtA, lgtC and lgtD) was genetically fixed either on or off (or deleted) as described in Chakraborti et al., 2016.

Strains for functional assays: A strain panel representative of the general Gonococcus population and including isolates expressing either the PorB 1a or the PorB 1b variants was selected for antibody functional testing by hSBA. Table 3 lists some of the features of the strain panel used for the functional assays. The strains used for the functional assays are representative of the phylogenetic population of Neisseria gonorrhoeae.

TABLE 3
Features of strains used for functional assays. Strains marked “INT”
form part of an internally sequenced collection.

| | | Serum | | Genome |
|---|---|---|---|---|
| Strain | PorB Variant | resistance (Y/N) | Country | Source |
| FA1090 | PorB1b | Y | N/A | PubMLST id # |
| | | | | 2855 |
| F62 | PorB1b | N | N/A | PubMLST id |
| | | | | #21070 |
| MS11 | PorB1b | N | USA | PubMLST id # |
| | | | | 27228 |
| BG27 | PorB1b | Y | UK | INT |
| WHO-M | PorB1b | N | Australia | EMBL-EBI |
| | | | | (ENA) # |
| | | | | LT591904 |
| BG8 | PorB1b | Y | UK | INT |
| GC14 | PorB1b | N | Canada | INT |
| SK92-679 | PorB1a | Y | USA | PubMLST id # |
| (“SK92”) | | | | 21071 |
| WHO-F | PorB1a | Y | Canada | EMBL-EBI |
| | | | | (ENA) # |
| | | | | LT591897 |
| WHO-G | PorB1a | Y | Thailand | EMBL-EBI |
| | | | | (ENA) # |
| | | | | LT91898 |
| WHO-N | PorB1a | Y | Australia | EMBL-EBI |
| | | | | (ENA) # |
| | | | | LT591910 |

Strains were routinely cultured at 37° C. in an atmosphere of 5% CO₂ on Gonococcus (GC) agar medium (Difco) plates enriched with 1% v/v of Isovitalex. For growth in liquid cultures, bacteria grown for at least 18 h on the plates were diluted to an OD_{600 nm} of 0.3-0.4 in liquid GC—1% v/v Isovitalex and incubated at 37° C. at 160 rpm.

To induce pilus expression and enable transformation, Neisseria gonorrhoeae strains were cultured on GC agar plates supplemented with 0.25 mM (IPTG). Chloramphenicol was added to achieve a final concentration of 10 g/ml for selection of transformed clones.

Generation of Neisseria gonorrhoeae Mutants

Generation of DNA construct for lgtF knockout (KO): A number of the experiments described in the Examples below include OMVs isolated from Neisseria gonorrhoeae (FA1090) that have been genetically manipulated by knocking out lgtF. The resulting gonococcal mutant no longer expresses any sugars on its alpha-chain.

The lgtF KO was obtained by genomic recombination where the coding sequence of this enzyme was replaced with the chloramphenicol antibiotic resistance cassette (lgtF::cmR) with a double crossing over. The amplification of 515 bp upstream and 548 bp downstream regions of the lgtF gene was performed with the primer couples lgtF-UP-fw/rv and lgtF-DO-fw/rv respectively using as template 50 ng of genomic DNA purified from the FA1090 wild-type strain and KapaHiFi DNA polymerase (Roche); the amplification of the chloramphenicol resistance gene (cmR) was done with the primer couple cloKOF/R using 10 ng of a synthetic DNA template (Geneart). Primer sequences are listed in Table 4 below. Primer tails indicated in italics were added to allow Polymerase Incomplete Primer Extension (PIPE) cloning (Klock et al. Methods Mol Biol. 2009; 498:91-103) of PCR products into a modified pET15 vector, previously amplified with primer couple PIPEfw/pET 15KOrv. According to PIPE method, PCR products were transformed into E. coli MACH-1 competent cells (Thermo Scientific) immediately following amplification. Screening of transformants was done by colony PCR using T/prom/pETseqRv primers. The PCR product from positive clones, corresponding to DNA fragment lgtF::CmR was purified using the QIAquick PCR purification kit (QIAGEN) following the manufacturer's protocol and used for gonococcus transformation.

TABLE 4
List of primers used for the generation of lgtF
knockout in N. gonorrhoeae FA1090 ΔlpxL1 strain

| | | SEQ ID | |
|---|---|---|---|
| Name | Sequence 5′-3′ | NO: | Use |
| lgtF-UP-fw | ATAGGGGAATTGTGCTCGAGACGCACCAC | 13 | Amplification of lgtF |
| | AACGACAGTATGGAAAG | | upstream region |
| lgtF-UP-rv | TCCTTCAGACGGCATTCCCGGGGGTTTCT | 14 | |
| | CAAAGCATTTTGGTTTC | | |
| lgtF-DO-fw | GGATCCCCATGGATACCCGGGCAAACTAT | 15 | Amplification of lgtF |
| | ATTATCTGTACAAATCC | | |
| lgtF-DO-rv | AATTAAGTCGCGTTATCTAGAGTATCGATA | 16 | downstream region |
| | CCGTTATGAACAATCC | | |
| cloKOF | ATGCCGTCTGAAGGATCCGTCAACCGTGA | 17 | Amplification of cmR |
| | TATAGATTGAAAAGTG | | |
| cloKOR | TATCCATGGGGATCCGATCCACGCGTCTT | 18 | cassette |
| | AAGGCGG | | |
| PIPE-Fw | TAACGCGACTTAATTGGCCAGTGTGCCG | 19 | Amplification of cloning |
| | GTCTCCG | | |
| pET15KOrv | TCACAATTCCCCTATAGTGAGTC | 20 | vector |
| T7Prom | TAATACGACTCACTATAGGG | 21 | Screening for cloning |
| pETseqRv | GATATCCGGATATAGTTCCTC | 22 | |
| lgtF-ext-F | AAGACATCGGCCGGATTAATC | 23 | Mutant screening |
| lgtF-ext-R | GACGGTAAAAAAACGGCTGTCGG | 24 | |
| lgtF-wt-F | CTTCCATCTCGGCCGAGAAATACC | 25 | Check wild-type locus |

Generation of lgtF knockout in N. gonorrhoeae FA1090 Δlpxl1 strain: N. gonorrhoeae FA1090 Δlpxl1 strain was previously generated. An example of the method used for generation of an Δlpxl1 strain may be found, for example, in Zollinger et al (2010) Vaccine. 28(31):5057-67. Its transformation with the PCR product obtained above from recombinant E. coli was carried out and transformants were selected into GC agar plates+1% Isovitalex with chloramphenicol 2 g/ml. Transformants were tested by PCR analysis using Accuprime Taq Polymerase (Thermo Scientific) and with external lgtF-ext-F/R to check the correct event of double recombination leading to the deletion of the lgtF gene. The expected band for the mutant strains with cmR (chloramphenicol resistance cassette) is about 1960 bp while the wild-type locus is 1860 bp long. Positive clones were streaked repeatedly onto GC agar plates+1% Isovitalex with chloramphenicol 3 g/ml; glycerol stock and DNA lysates were collected at each passage and tested for the presence of remaining wild-type population. PCR screenings were performed using Accuprime Taq Polymerase (Thermo Scientific) with the internal primer lgtF-wt-F, specific for the wild-type DNA in combination with the external primer lgtF-ext-R. Clones presenting the lgtF deletion yielded no PCR products, while those expressing the wild-type gene gave a 767 bp long PCR product.

Generation of rmp knockout in N. gonorrhoeae FA1090 ΔlpxL1 ΔlgtF strain: The DNA constructs rmp::eryR for mutants generation was previously prepared from pBSRMPKO with primers UpIII-FOR and DpIII-REV (see Table 5 below) and it was used as a template for the amplification of the DNA needed for the transformation. The PCR was performed using the primers UpIII-FOR and DpIII-REV and the KAPA HIFI 2× master mix (Roche), with reaction conditions as follows: 95° C. for 5 min, 25 cycles of 98° C. for 30 s, 55° C. for 30 s and 72° C. for 3 min 30 s, with a final step at 72° C. for 7 min. DNA purifications were performed using the KIT wizard SV Gel and PCR clean up system (Promega) following the manufacturer's protocol. The PCR product corresponding to the DNA fragment rmp::eryR, was used for the transformation of the FA1090 ΔlpxL1, ΔlgtF strain. Transformations were carried out by spotting a mixture of bacterial resuspension in PBS and DNA onto a GC agar plate and by incubating it for 5-6 hours. Transformants were selected into GC agar plates with erythromycin 2 g/ml to select the Δrmp. All transformants were tested by PCR analysis using Accuprime Taq Polymerase (Thermo Scientific) and with external primers (primer couples UP_CHECK_NG01577-Fw/DW_CHECK_NGO1577-Rev) to check the correct event of double recombination. The expected a band for the mutant strain with eryR (erythromycin resistance) is about 1900 bp while the wild-type locus is about 1400 bp long. Positive clones were streaked repeatedly onto selective agar plates; glycerol stock and DNA lysates were collected at each passage and tested for the presence of remaining wild-type population. PCR screenings were performed using Accuprime Taq Polymerase (ThermoFisher) and with the internal primer INTwt_NGO1577-Fw, specific for the wild-type DNA, in combination with the external primer DW_CHECK_NG01577-Rev. The desired clones tested by PCR using the primer couples didn't yield any PCR product confirming the successful rmp deletion, while those expressing the wild-type rmp gene gave a PCR product of about 900 bp.

TABLE 5
Primers used for the generation of rmp
knockout in N. gonorrhoeae FA1090 ΔlpxL1
ΔlgtF strain

| Name | Sequence | SEQ ID NO |
|---|---|---|
| UpIII-FOR | gctctagaGGTCGT | 26 |
| | CTATCCGTTCCGTA | |
| DpIII-REV | cccgctcgagGGGA | 27 |
| | AAGGCGTGAATTTG | |
| | TA | |
| UP_CHECK_ | GTGTGTCCAGTCGT | 28 |
| NGO1577- | AGCAGG | |
| Fw | | |
| DW_CHECK_ | AGGGATGATGATAA | 29 |
| NGO1577- | AACCATATCC | |
| Rev | | |
| INTwt_NGO1577- | TCGTACGCAACAAC | 30 |
| Fw | TATGGAG | |
| INTwt_NGO1577- | CATCAACATATTGA | 31 |
| Rev | GGAGCCTG | |

Generation of DNA construct for the lpxl1 knockout: All DNA primers used to generate the LpxL1 knockout mutants are reported in Table 6. The plasmidic DNA containing the synthetic construct ΔLpxL 1-cmR-PheS (construct3PHS) was received from Geneart. The linearized cassette ΔlpxL1-cmR-PheS, which contains the cloramphenicol resistance gene and the upstream and downstream regions for the homologous recombination, was used as a template for the amplification of the DNA needed for the transformation. The PCR was performed using the primers Lpx UP Fwd and LpxL1 DO Rev and the KAPA HIFI 2× master mix (Roche), with reaction conditions as follows: 95° C. for 5 min, 30 cycles of 98° C. for 30 s, 60° C. for 30 s and 72° C. for 3 min 30 s, with a final step at 72° C. for 7 min. DNA purifications were performed using the KIT wizard SV Gel and PCR clean up system (Promega) following the manufacturer's protocol.

TABLE 6
List of primers used for the generation of
lpxL1 knockout mutants in N. gonorrhoeae
MS11 v.4/3/1 LOS mutant strains

| | | SEQ |
|---|---|---|
| | | ID |
| Name | Sequence | NO |
| Lpx UP Fwd | GGCATTTGTATTTTGCCGTCTG | 32 |
| LpxL1 DO Rev | CGCCATTTTCTACGCTTTGCCAAG | 33 |
| NGO_lpxL1_ | CCGCGTTCGAGATGG | 34 |
| wtcheck-Fw | | |
| NGO_lpxL1_ | GCGGAACTGTTTGACGAG | 35 |
| wtcheck-Rev | | |
| LpxL1 est FW | CCGCCAAACTCAATCCTTCG | 36 |
| LpxL1 est REV | GCAAACTTTTGTTTCACCGTTTCCG | 37 |

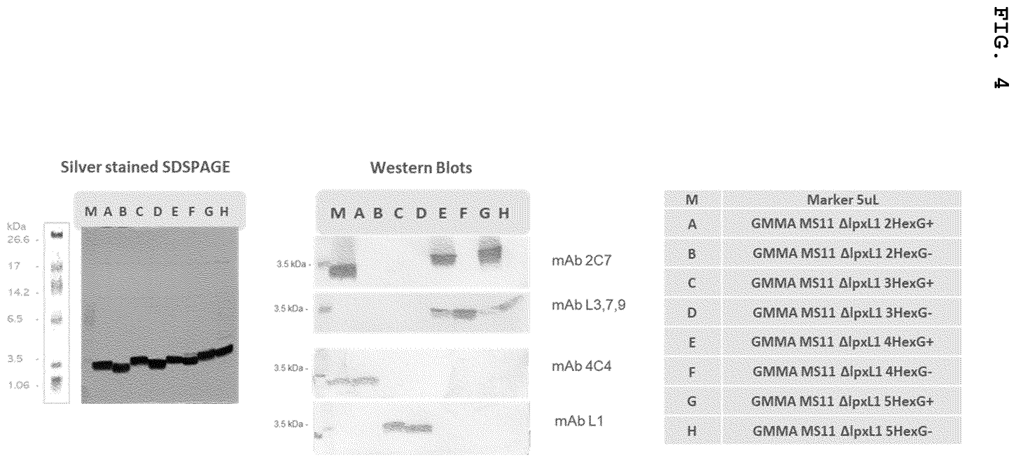

Generation of lpxL1 knockout in N. gonorrhoeae MS11 v.4/3/1 lgt mutant strains: LOS mutant strains whereby the expression of the four phase-variable lgt genes (lgtG, lgtA, lgtC and lgtD) was genetically fixed either on or off (or deleted) were previously generated. A schematic representation of the resulting LOS structure of each strain is reported in FIG. 3. In order to reduce LOS endotoxin activity, the lpxL1 gene (NGO0154) was genetically deleted in each lgt mutant strain. The lpxL1 KO was obtained by genomic recombination where the coding sequence of this enzyme was replaced with the chloramphenicol antibiotic resistance cassette (lpxL1::cmR) with a double crossing over. The PCR product, the linearized plasmidic DNA ΔLpxL1-cmR-PheS, was used for the transformation of all the MS11 LOS mutant strains. Transformations were carried out by spotting a mixture of bacterial resuspension in PBS and DNA onto a GC agar plate and by incubating it for 5-6 hours. Transformants were selected into GC agar plates with chloramphenicol g/ml to select the Δlpxl1. All transformants were tested by PCR analysis using Accuprime Taq Polymerase (Thermo Scientific) and with external primers (primer couples LpxL1 est FW/REV) to check the correct event of double recombination. The expected band for the mutant strain with cmR (chloramphenicol resistance) is about 2986 bp while the wild-type locus is about 1703 bp long. Positive clones were streaked repeatedly onto selective agar plates; glycerol stock and DNA lysates were collected at each passage and tested for the presence of remaining wild-type population. PCR screenings were performed using Accuprime Taq Polymerase (ThermoFisher) and with the external primer LpxL1 est FW in combination with the internal primer NGO_lpxL1_wtcheck-Rv, specific for the wild-type DNA. The desired Clones tested by PCR using the primer couples didn't yield any PCR product confirming the successful lpxL1 deletion, while those expressing the wild-type lpxL1 gene gave a PCR product of about 625 bp.

### LOS Extraction

LOS was extracted using a phenol-water procedure from bacterial pellet or directly from OMV samples. Bacterial pellets were obtained after centrifugation of the gonococcal strain grown in liquid culture to exponential phase. The pellet was suspended in a buffer (6 mM tris-base 10 mM EDTA 2% SDS, pH 6.8) containing 50 g/ml of proteinase K and stirred at 65° C. for 2 hours. The sample was then placed overnight at 37° C. A solution of sodium acetate ( 1/10 of the sample volume) was added together with 3 volumes of cold ethanol. After centrifugation at 12000×g for 10 min at 4° C., the supernatant was removed, and the pellet suspended in sodium acetate buffer. Cold ethanol precipitation was repeated 3 times before the pellet was suspended in a buffer containing 10 mM Tris base, 100 mM NaCl, 5 mM CaCl₂, pH 7.3, MgCl₂ 10 mM and 100 μL of benzonase (Merk, 250 U/μL). The sample was stirred overnight at 37° C. and 50 rpm.

LOS was extracted with a hot phenol/water procedure from bacterial pellets treated as described above or from OMV particles obtained as described below. The bacterial suspension or OMVs were stirred at 65° C. until the temperature equilibrated. An equal volume of 90% (w/v) phenol which had been preheated to 65° C. was added and thoroughly mixed for 30 minutes. The resulting mixture was rapidly cooled by stirring for 30 minutes in an ice-water bath. The phenol mixture was then centrifuged at 4° C. at 4000×g for 10 minutes. A sharp interface occurs between the aqueous, phenol, and interface layers. The aqueous and phenol layers were removed by aspiration. The aqueous layer containing the lipopolysaccharide was retained while the phenol layer was discarded. Cold ethanol precipitation was performed 3-4 times and the final pellet was suspended in distilled water and ultracentrifuged at 175000×g for 3 hours. After ultracentrifugation, the pellet containing extracted LOS was suspended in distilled water.

### OMV Production and Purification

Bacterial Fermentation: For each batch, the production of biomass started from a frozen working bacterial stock by growth in plate for at least 12-16 h. After growth, this was sub-cultured to a pre-culture, that was subsequently used to inoculate the production medium into a shake flask in liquid GC—1% v/v Isovitalex supplemented with 5 g/L Na-Lactate, 2.5 g/L Na-Glutammate, 0.5 g/L Serine, 0.3 g/L Cysteine. The culture was incubated at 37° C. 180 rpm, starting from an optical density measured at 600 nm (OD600) of 0.3-0.4. The bacterial culture was grown until it reached an OD600 equal to 1.5±0.5, usually in 9±2 hours.

OMV Isolation and Purification: The collected bacterial culture was centrifuged at 12000×g for 30 min at 4° C. in order to remove the bacteria and large debris from the solution. The supernatant, containing the vesicles released into the fermentation broth, was then carefully recovered and filtered with a 0.2 μm PES filter to sterilize and further remove large debris. Then, 400 U/L Benzonase (Merck) (2 h, 37° C.) was added to the crude OMVs to digest DNA following the manufacturer's instructions and subsequently the supernatant was filtered again with a 0.2 m PES filter. Next, the solution was concentrated and diafiltrated using tangential flow filtration (TFF). The suspension was concentrated using a 200 cm2 300 kDa cut-off PESU membrane (SARTOCON SLICE 200 Sartorius stedim polyethersulfone 300 kDa), followed by 25 volume diafiltration with buffer (PBS 1×) to wash out the original buffer salts (or other low molecular weight species) in the retentate. The retentate was then concentrated again and then washed with 40 volumes of PBS 1×. When purity levels were below 80% (established by SE-HPLC), the sample was further purified by centrifugation at 150000×g (Optima L90K, rotor SW31Ti S/N 15U4385, tubes Ultraclear 38.5 mL P/N 344058) for 2 h and solubilization in sterile PBS 1× buffer for 24 h at 4° C. in a laboratory tilting shaker. Lastly, the purified OMVs were filtered using a 0.2 μm cut-off filter to obtain sterile samples.

### Determination of LOS Structures

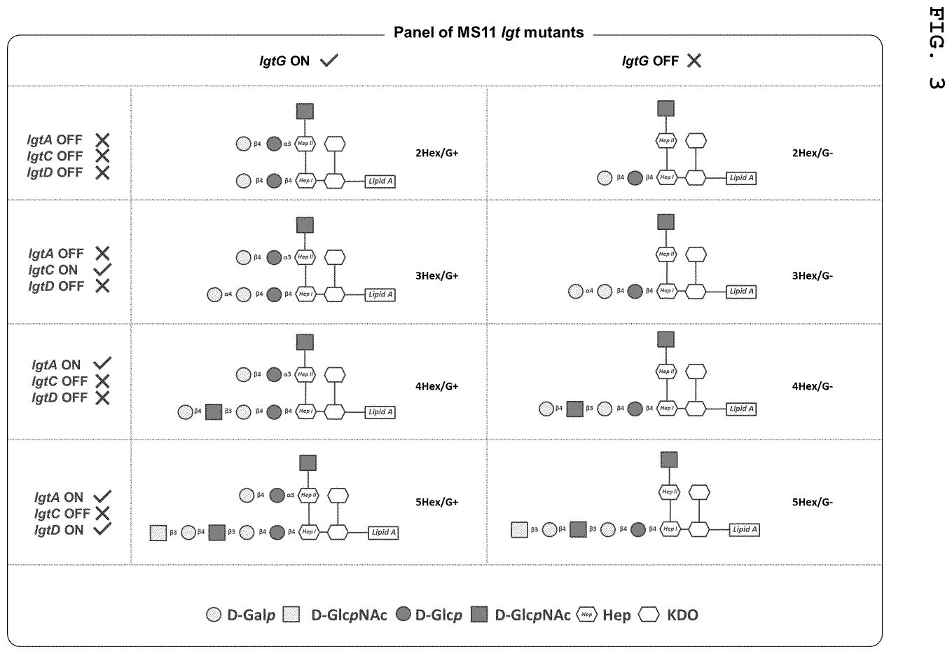

Antibodies: Anti-LOS mAb 17-1-L1 (henceforth referred to as mAb L1) (Griffiss et al. J. Biol. Chem. 2000, 275: 9716-9724), 4C4 (Dudas et al. Infect. Immun. 1988, 56: 499-504), L3,7,9 (Griffiss et al. J. Biol. Chem. 2000, 275: 9716-9724) and 2C7 (Gulati et al. J. Infect. Dis. 1996, 174: 1223-1237) have been described previously. A schematic representation of the epitopes recognized by these mAbs is provided in FIG. 2. The tissue culture supernatant containing anti-LOS mAbs L1 was kindly provided by Professor Sanjay Ram (Division of Infectious Diseases and Immunology, University of Massachusetts Medical School). Mouse mAb 2C7 has been produced as recombinant internally. Purified mAb 4C4 and supernatant of mAb L3,7,9 are commercially available (Novusbio).

Western blotting: The samples of LOS or OMVs were titrated in previous WB experiments to check the optimal quantity of samples that can permit a clear LOS profiling with well separated LOS bands that correspond to different LOS structure populations (data not shown). Loading of OMVs into each well of an SDS-PAGE gel was normalized based on LOS quantity obtained from semicarbazide-HPLC method: 0.08 nmol KDO/well. Samples were run on a 16% Tris-glycine SDS-PAGE gel using Tris-glycine 1× buffer. The marker used was Ultra-Low range and consisted of the following protein markers: Bradykinin (1060 Da), Insulin Chain B (3496 Da), Aprotinin (6500 Da), α-Lactalbumin (14200 Da), Myoglobin (17000 Da) and Triosephosphate Isomerase (26600 Da). LOS was transferred to nitrocellulose membranes (iBlotKit, Thermofisher) and membranes were blocked with PBS 1×+ BSA 3%+Tween20 0.05% for 1 h at RT. Anti-LOS mAbs (diluted 1:1000 in PBS 1×+Tween20 0.05%) were incubated with membranes for 1 h at RT. mAb-reactive LOS bands were visualized with anti-mouse IgG alkaline phosphatase (diluted 1:2000 in PBS 1×+Tween20 0.05%) incubated for 30 minutes at RT, followed by AP Conjugate Substrate kit (Biorad) for 5 minutes at RT.

Silver stained SDS-PAGE: The samples of LOS or OMVs were titrated in previous silver staining experiments to check the optimal quantity of samples that can permit a clear LOS profiling with well separated LOS bands that correspond to different LOS structure populations (data not shown). Loading of OMVs on each well of SDS-PAGE gel was normalized based on LOS quantity obtained from semicarbazide-HPLC method: 0.12 nmol KDO/well. The SDS-PAGE gel was a 16% Tris-glycine gel (Invitrogen), and samples were run with Tris-glycine 1× buffer (Invitrogen). The marker was Ultra-Low range and consisted of the following protein markers: Bradykinin (1060 Da), Insulin Chain B (3496 Da), Aprotinin (6500 Da), α-Lactalbumin (14200 Da), Myoglobin (17000 Da) and Triosephosphate Isomerase (26600 Da). After running, gels were fixed with fixation solution (40% ethanol, 5% acetic acid, 55% water) for 30 seconds in microwave oven (700 W) and after were incubated for 5 minutes at room temperature. Gels were then oxidized with 0.07% NaIO₄ in the same fixation solution and left for 5 minutes in the darkness. Gels were washed with a solution of 30% ethanol for 5 minutes after 30 seconds in microwave oven then were stained using Silver Quest Staining kit (Thermo Fisher cod LC6070) according to manufacturers recommendations.

Hydrophilic interaction chromatography (HILIC) coupled with Mass spectrometry: Prior to all chromatographic analysis, LOS or OMV samples were hydrolyzed by Acetic Acid (final concentration 1%) for 2 hours at 90° C. After hydrolysis samples were chilled for 15 minutes at 4° C. and transferred into clean 1.5 mL Eppendorf vials. Hydrolysis vials were washed twice with 200 μL of water. This water was added to the Eppendorf vials containing the samples and was centrifuged at 14000×g for 10 minutes. The supernatants, containing OS in solution, were separated from the pellet (Lipid A, proteins etc.), moved to clean vials and evaporated in speedvac. Samples were reconstituted with water to obtain the desired OS concentration.

To obtain UV detectable samples were derivatized with Semicarbazide (SCA). A stock SCA solution was prepared dissolving 100±2 mg of SCA Hydrochloride and 90.5±2 mg of Sodium Acetate in 10 mL of water. Equal volumes of sample and SCA solution were transferred into clean 1.5 mL Eppendorf vials (e.g., 100 μL sample+100 μL SCA solution) and heated at 50° C. for 50 minutes in a water bath. Samples were chilled at 4° C. for 15 minutes and filtered into HPLC vials.

Mass spectrometry was performed by QToF Premier (Waters) Quadrupole-Time of Flight Tandem Mass Spectrometer complete with Z-Spray Atmospheric Pressure Ionization Source, Modular Lock spray with ESI probe. The sample was delivered to spectrometer by Acquity UPLC system equipped with Binary solvent manager, Sampler manager and Column manager. The chromatographic separation has been performed by using two different gradients elution described below.
Autosampler Compartment: 12° C.
Column Compartment: 60° C.
Column: Guard Column BEH Glycan 1.7 μm (Part No186004739)+Column Acquity UPLC BEH Glycan 1.7 μm 2.1×150 mm (Part No 186004742)

### Method 1

### Eluents:

Line A: 100 mM Ammonium Formate pH 4,5
Line B: Acetonitrile

### Gradient 1:

| Time | Flow (mL/min) | % A | % B | Curve |
|---|---|---|---|---|
| 0, 0 | 0, 4 | 5 | 95 | 6 |
| 1, 0 | 0, 4 | 5 | 95 | 6 |
| 9, 0 | 0, 4 | 38 | 62 | 6 |
| 18, 0 | 0, 4 | 56 | 44 | 6 |
| 18, 5 | 0, 4 | 30 | 70 | 6 |
| 19, 0 | 0, 4 | 5 | 95 | 6 |
| 27, 0 | 0, 4 | 5 | 95 | 6 |

Curve 6 = linear

### Method 2

### Eluents:

Line A: 50 mM Ammonium Formate pH 4,5
Line B: Acetonitrile

### Gradient 2:

| Time | Flow (mL/min) | % A | % B | Curve |
|---|---|---|---|---|
| 0, 0 | 0, 4 | 75 | 25 | 6 |
| 45, 0 | 0, 4 | 54 | 66 | 6 |
| 46, 5 | 0, 2 | 0, 0 | 100 | 6 |
| 49, 5 | 0, 2 | 0, 0 | 100 | 6 |
| 53, 1 | 0, 2 | 75 | 25 | 6 |
| 57, 6 | 0, 4 | 75 | 25 | 6 |
| 65, 0 | 0, 4 | 75 | 25 | 6 |

Curve 6 = linear

The mass analysis was performed both in positive and negative mode applying conditions slightly different according with response of each sample.
Example of setting used for negative polarity: Analyser V Mode, Np Multiplier 0.70, Resolution 8000, Trigger Threshold 700, Signal Threshold 35, TO Constant-50.0, Capillary (kV) 2.8, Sampling Cone 45.0, Extraction Cone 4.0, Ion Guide 2.5, Source Temperature (° C.) 120, Desolvation Temperature (° C.) 300, Cone Gas Flow (L/Hr) 50.0, Desolvation Gas Flow (L/Hr) 800.0, LM Resolution 5.0, HM Resolution 15.0, Ion Energy 1.0, Pre-filter 2.0, Collision Energy 6.0.
Example of setting used for positive polarity: Analyser V Mode, Np Multiplier 0.70, Resolution 8000, Trigger Threshold 700, Signal Threshold 35, TO Constant-50.0, Capillary (kV) 3.0, Sampling Cone 40.0, Extraction Cone 4.0, Ion Guide 2.0, Source Temperature (° C.) 120, Desolvation Temperature (° C.) 300, Cone Gas Flow (L/Hr) 50.0, Desolvation Gas Flow (L/Hr) 800.0, LM Resolution 5.0, HM Resolution 15.0, Ion Energy 1.0, Pre-filter 2.0, Collision Energy 6.0.

### Functional Assays

Functional antibodies measured by Serum Bactericidal Assay: Functional antibodies were measured by human Serum Bactericidal Activity assay (hSBA) on strains FA 1090, BG27, BG8, SK92-679, WHO-F, WHO-G, WHO-N, F62, MS11, WHO-M and GC14 using normal human serum as complement source. The hSBA was performed on sera from animals immunized with different OMV vaccine preparations (collected 2 weeks after the second immunization).

Bacteria were plated on a round GC+1% Isovitalex agar plate and incubated 16 (+2) hours at 37° C. with 5% CO₂. The day after, single colonies were inoculated in GC+1% Isovitalex medium (CMP-NANA was added to the broth medium for serum sensitive strains: 0.5 ug/mL for F62 and GC14 strains, 0.2 ug/mL for WHO-M and MS11 strains) and incubated at 37° C. at 180 rpm until the culture reached OD600 nm=0.4-0.5. Bacteria were then diluted 1:10000 in SBA buffer (DPBS, 1% BSA, 0.1% Glucose) except for BG27 bacteria that were diluted 1:2500. Mouse sera, previously heat inactivated at 56° C. for 30 minutes, were serially diluted (ten 2-fold dilution steps) in SBA buffer. The assay was assembled in a sterile 96 flat bottom well microplate in a final volume of 32 μl/well. The serial dilutions of each test sample were let to react with bacteria and human complement. The following volumes and concentrations of each reaction component (table 7) were added in order:

TABLE 7
Reaction mixture volumes for each component used in hSBA.

| | Serum resistant strains | |
|---|---|---|
| | (FA1090, BG27, BG8, | |
| Reaction | SK92-679, | Serum sensitive strains |
| mixture | WHO-F, WHO-G, | (F62, MS11, WHO-M, |
| Volumes | WHO-N) μl/well | GC14) μl/well |
| Test sample | 16 | 16 |
| Bacteria | 11 | 9.5 |
| Human | 5 (final | 6.5 (pre- |
| complement | concentration | diluted 1:2 in SBA |
| | 16%/well) | buffer, final |
| | | concentration 10%/well) |

Each plate also included the following controls:
Heat inactivated complement (HIC) controls: all serum samples tested in the presence of bacteria and HIC. This control allows to exclude serum toxicity.
Without (w/o) serum control: bacteria with active human complement (AC) in absence of serum sample. This control was used to exclude complement toxicity and to determine 100% bacterial growth.
A hyper-immune serum: pooled serum from mice receiving a vaccine known to induce strong positive hSBA titers (included as positive control).

The reaction mixture was incubated at 37° C. for 60 minutes at 160 rpm. After 60 minutes of incubation (T60) 100 μl/well of agar overlay medium were added in each well. After agar addition, the microplates were incubated overnight at 37 C° with 5% CO₂. The day after the plates were automatically acquired with the image analysis system DISCOVERY V12 AXIOLAB. The CFUs in each 96-well of plate were counted using the image analysis system (Reading AxioVison). The bactericidal titer for each test sample was calculated as the reciprocal of the serum dilution giving a killing>50% with respect to the average number of CFU calculated on the 8 replicates of w/o serum control at T60 (average CFU w/o serum ctrl). Where more than one serum dilution gives 50<killing<55%, the lowest dilution is chosen to calculate the hSBA titer.

For some experiments after the mixture incubation at 37° C. for 60 minutes at 160 rpm the reactions from each well were plated manually: 7 l/well were plated onto square GC+1% Isovitalex plates. Colonies were counted after overnight incubation at 37° C. 5% CO2 in humid atmosphere.

Bacterial Adhesion Inhibition (BAI) Assay: A cell-based fluorescent BAI assay was used to assess the capability of murine sera raised against OMVs derived from MS11 mutants, to inhibit the adhesion of gonococcus to SV-HUC1 cell line (a human ureteral epithelial cell line). Briefly, monolayer of SV-HUC1 cells were detached from 175 cm2-flask and cell number and viability were determined by the Countess Automated Counter. SV-HUC1 were seeded into 96 wells plates (3×105 cells/well) and cultivated in F-12K Nut Mix medium for 4 days to allow the cell culture to reach confluence. Gonococcal strains were harvested from a fresh overnight plate culture into 10 mL of GC+1% Isovitalex medium. Bacteria were grown at 37° C. under shaking until A600=0.5, then resuspended in DPBS and labelled with Oregon Green dye for 15 minutes at 37° C. Afterwards bacteria were washed to remove excess of dye and combined, at final A600=0.1, with an equal volume of serially 1% BSA-DPBS diluted sera for 15 minutes at room temperature. Bacteria-sera complexes were then added to cell plate and incubated for 1 hour at 37° C. to allow bacteria-cells adhesion. After 3 washes with DPBS, samples were fixed 20 minutes with 4% formaldehyde at RT and, after one washing step with DPBS, finally 1 volume of distilled water was added to each well. Plates were analysed by Opera Phenixv (PerkinElmer) instrument. In detail, 9 fields of each well were acquired with a 40× water objective. For each field, a minimum of 10 Z-planes were acquired and analyzed in maximum projection mode: for each pixel the higher fluorescence value among all the Z planes acquired was plotted into a new calculated projected image. Afterwards an algorithm was applied in order to extrapolate the total volume of fluorescent bacteria so that the mean fluorescence measure of adhering bacteria for each well was obtained (raw data referred as All bacteria Volume).

To obtain the % of inhibition the raw data were transformed by the following formula:

$$100 - \frac{Bacteria Volume}{Mean bacteria volume alum} * 100$$

where Bacteria Volume was each single raw data value and Mean bacteria volume alum was the mean of the Bacteria Volume observed for selected Alum dilutions. Only lots that achieved a 30% bacterial adhesion inhibition success criterion was subjected to statistical analysis.

Competitive hSBA: A fixed dilution of the tested sera pool (i.e. sera obtained from mice immunized with OMV vaccine) was incubated 1:1 (vol/vol) with 3 different concentrations (nmol LOS/ml) of each competitor for 1 h at 37° C., 180 rpm. The same pool was also incubated 1:1 (vol/vol) with SBA buffer to measure the hSBA titer of “NOT INHIBITED” sample. After 1 hour of incubation, the mixture serum-competitor was dispensed in plate, diluted 1:2 for eleven dilution-steps and then bacteria and human complement were added following the hSBA assay protocol (see above). The “without serum” control (8 wells) was included in each plate (i.e. bacteria with active human complement (AC) in absence of serum sample). This control was used to exclude complement toxicity and to determine 100% bacterial growth. The competition experiments were performed on the strains FA 1090, WHO-M, WHO-G, WHO-N, F62 and MS11.

### Example 2: Determination of LOS Structures

A number of techniques were used to determine the LOS structures present in the different OMV preparations described herein. An immunochemical characterization was made by western blotting (WB) and silver staining of samples run by SDS-PAGE, whereby probing with anti-LOS mAbs which bind to specific LOS epitopes provided insight into which alpha/beta-chain oligosaccharide structures were present. Silver staining of LOS provided an insight into which LOS structures were present depending on size-dependent migration of LOS bands relative to a 3.5 kDa marker band. Following immunochemical characterization, the specific LOS structures present in a given OMV preparation was confirmed by mass spectrometry. Examples of these analyses are provided below:

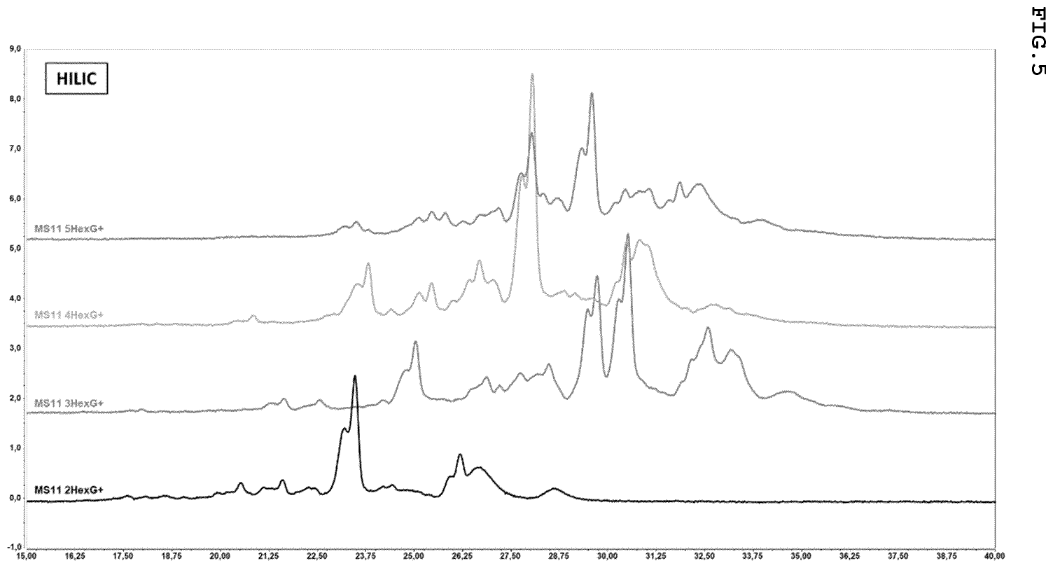

MS11 Mutant Strains: The LOS structures displayed on OMVs isolated from 8 MS11 lgt mutant strains (FIG. 3) was confirmed by silver staining and western blot using four different LOS specific mAbs. SDS-PAGE silver-stained and corresponding WB using different LOS specific mAbs for OMVs of MS11 mutant strains are reported in FIG. 4.

Silver staining the LOS from OMVs isolated from each mutant strain demonstrated molecular weight dependent migration of silver stained LOS bands. The LOS bands appear near the Insulin Chain B (3496 Da) band of the marker (both below and above) and therefore is considered a reference for the height of the LOS bands. OMVs displaying long alpha-chain structures (4HexG−, 4HexG+, 5HexG−, 5HexG+) appear above the 3.5 kDa marker whereas shorter alpha-chain structures (2HexG−, 2HexG+) resolve below the 3.5 kDa marker. 3Hex structures are better identified via western blot staining with the L1 mAb.

The binding of mAbs to specific LOS epitopes enables determination of which epitopes (and thus which LOS structures) were displayed on the OMV. Taking the WB and silver staining data into account the following observations were made:
Bands recognized by mAbs L1 and 4C4 correspond to LOS structures with a short α-chain (3HexG+/−) and 2HexG− respectively.
Bands recognized by mAb L3,7,9 correspond to LOS structures with a long α-chain without β-chain (4HexG−).
Bands below the insulin chain B band of the marker and recognized by mAb 2C7 correspond to LOS structures with a short α-chain and presence of beta-chain (2HexG+).
Bands at the same height or above the insulin chain B band of the marker and recognized by mAb 2C7 correspond to LOS structures with a long α-chain and presence of beta-chain (4HexG+ and 5HexG+).

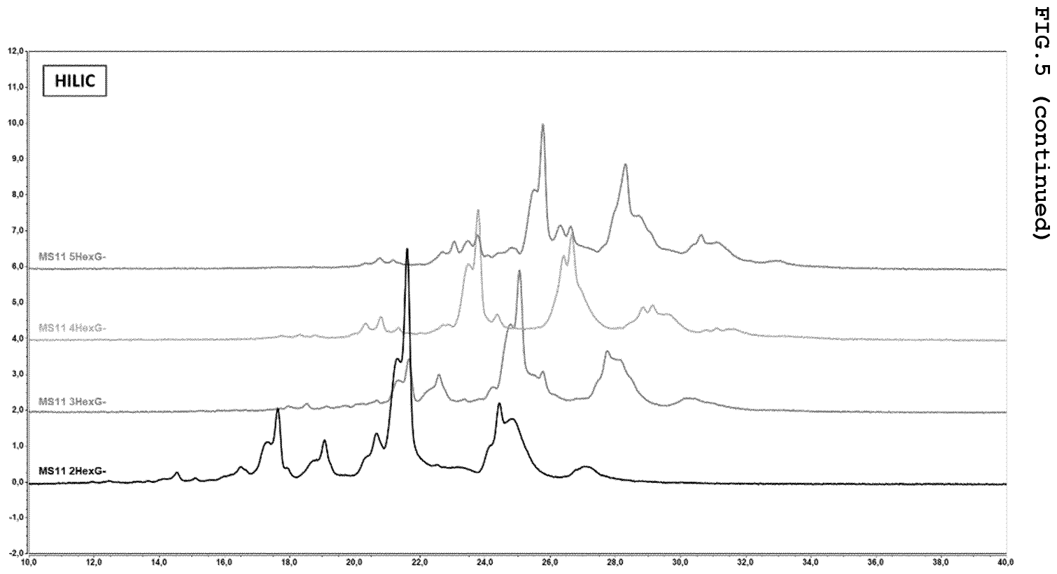

Finally, HILIC-MS revealed different profiles for MS11 mutants that expressed selected LOS populations (see FIG. 5). LOS Structures were determined by comparing the Mw determined experimentally via mass spectrometry with the calculated Mw for each possible LOS structure.

FA1090: The LOS structures displayed on the surface of OMVs obtained from FA1090 2KO (Δlpxl1, Δrmp) and FA1090 1KO (Δlpxl1) strains were determined using the techniques described above (silver staining, WB and mass spectrometry).

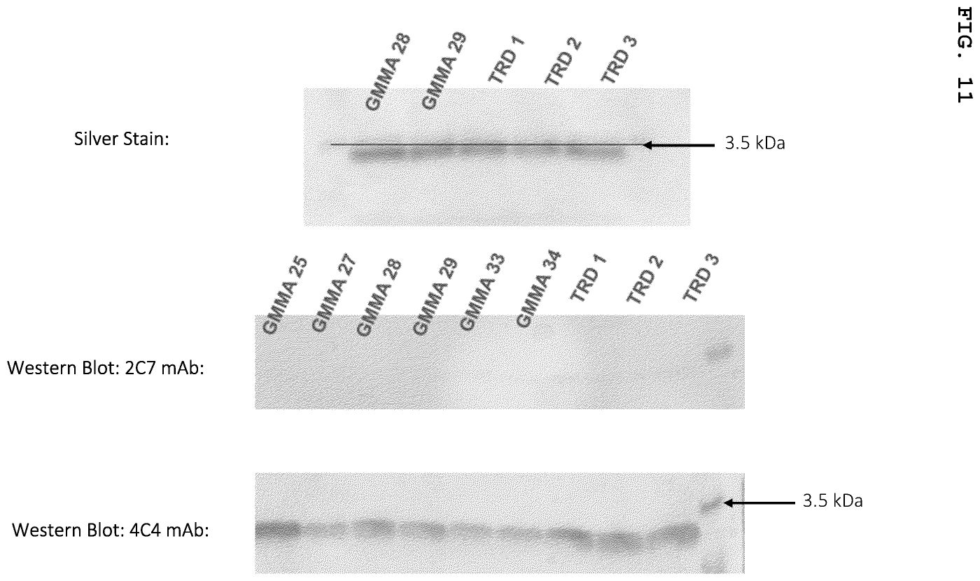

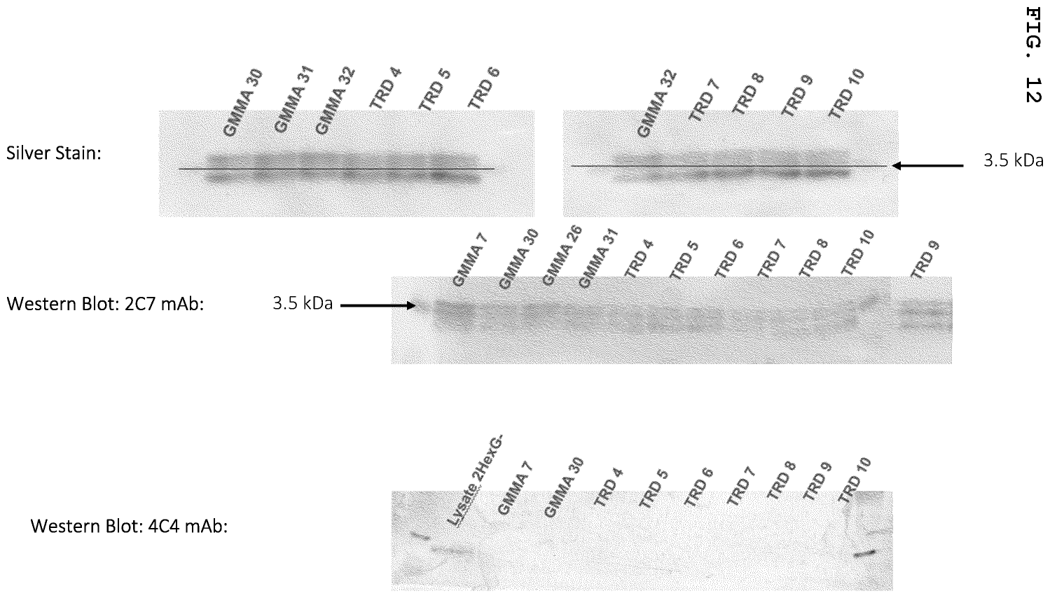

Western blot and silver staining of OMVs obtained from FA 1090 1KO (Δlpxl1) and 2KO revealed (Δlpxl1, Δrmp) revealed as follows:
1KO (Δlpxl1)—see FIG. 11. Only one band observed in silver staining (below the 3.5 kDa marker) consistent with highly truncated LOS. Western blot data demonstrates no binding of the 2C7 mAb (i.e. no beta-chain present, G−) and presence of mAb 4C4 binding. Thus it was concluded that the principle LOS structure present in FA1090 1KO (Δlpxl1) OMVs was the 2HexG− structure.
2KO (Δlpxl1, Δrmp)—see FIG. 12. Three bands were observed via silver staining only one of which appearing below the 3.5 kDa marker. This is consistent with three predominant LOS structures (one of which being highly truncated). Western blot data with the 2C7 mAb revealed the same three bands indicating the presence of 2HexG+, 4HexG+ and 5HexG+ structures. No binding was observed with the 4C4 mAb consistent with lack of 2HexG− structures.

In table 8 below are reported the main LOS structures detected by mass spectrometry for OMVs isolated from FA1090 2KO strains used herein. Because of the hydrolytic pretreatment of the sample and the possible in source fragmentation the shorter structures may be due to fragmentation of the longer ones when they are present at the same time. However this issue does not impact the immunochemical techniques (WB and Silver Stain) therefore using the mass spectrometry in combination with other techniques provided an accurate analysis of the LOS structures present.

All the structures indicated in the tables were assigned based on the accuracy of the experimental Mw compared to the calculated one.

TABLE 8
Main structures detected by mass spectrometry in FA1090 OMV and
purified LOS samples (2KO = (Δlpxl1, Δrmp).

| | | | | | | | | | | |
|---|---|---|---|---|---|---|---|---|---|---|
| | | | 2Hex/ | 2Hex/ | 3Hex/ | 3Hex/ | 4Hex/ | 4Hex/ | 5Hex/ | 5Hex/ |
| OMV | lot | production | G+ | G− | G+ | G− | G+ | G− | G+ | G− |

| | | | | | |
|---|---|---|---|---|---|
| 2KO | TRD4 | Fermentation | X | X | X |
| 2KO | TRD5 | Fermentation | X | X | X |
| 2KO | TRD7 | Fermentation | X | X | X |
| 2KO | TRD9 | Fermentation | X | X | X |
| Extracted | RC03Nov | Flask | X | X | X |
| LOS from | 21 | | | | |
| 2KO | | | | | |

Other Strains: Western blot and silver staining data is not shown for all strains but provided an initial insight into which LOS epitopes (and thus which LOS structures) were displayed on the surface of OMVS obtained from a number of other strains utilized herein. In table 9 below the structure detected by mass spectrometry for OMVs from other gonococcal strains is summarized.

TABLE 9
Main structures detected by mass spectrometry in OMVs from
other gonococcal strains. 1KO = Δlpxl1.

| | | | | | | | | | | | |
|---|---|---|---|---|---|---|---|---|---|---|---|
| | | | | 2- | 2- | 3- | 3- | 4- | 4- | 5- | 5- |
| | | | | Hex/ | Hex/ | Hex/ | Hex/ | Hex/ | Hex/ | Hex/ | Hex/ |
| Strain | OMV | lot | production | G+ | G− | G+ | G− | G+ | G− | G+ | G− |

| | | | | | | | |
|---|---|---|---|---|---|---|---|
| F62 | nOMV | GCnomv6 | Flask | | | X | X |
| | 1KO | GCgmma14 | Flask | | X | X | X |
| | 1KO | GCgmma22 | Fermentation | | X | X | X |
| SK92 | 1KO | GCgmma16 | Flask | X | | | |

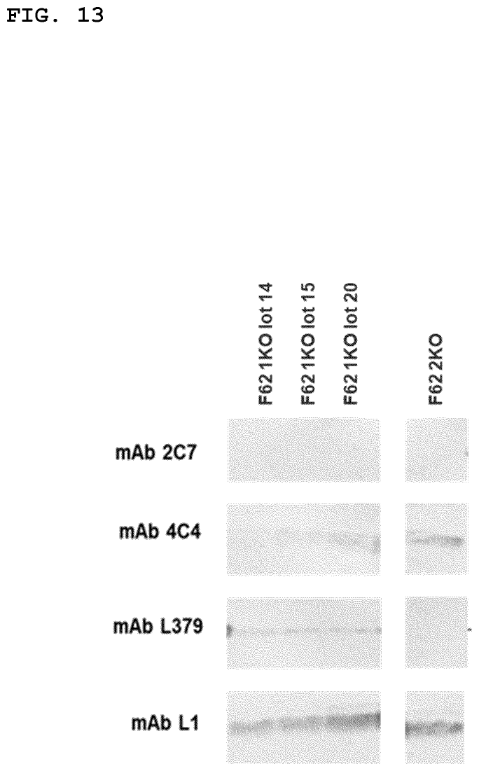

F62: Three batches of OMVs F62 1KO (Δlpxl1) and one batch of F62 2KO (Δlpxl1, Δrmp) were analyzed by WB experiments, showing changes in mAbs recognition by comparing 1KO to 2KO mutants (see FIG. 13). Notably, recognition of L3,7,9 mAb is lost in the F62 2KO mutant while a more intense band is observed with mAb 4C4, suggesting that F62 2KO shifts to LOS structures with a short α chain (2HexG− and 3HexG−). Mass spectrometry data for F62 2KO is not available.

In the following Examples the data showing the determination of the LOS structures is not shown for every OMV vaccine, however the LOS structures present (assigned according to the nomenclature of Table 1) has been provided.

### Example 3: Anti-LOS Antibodies Play Major Role in Cross-Bactericidal Immune Response Elicited by Gonococcal OMVs

Female CD1 outbred mice 7 weeks old (10/group) were immunized intraperitoneally 2 times at days 1, 29 with different lots of OMV-based vaccines (10 μg protein-based in 200 μl) adsorbed to alum (3 mg/ml), or Alum alone (200 μl).

Pooled sera were collected two weeks after the second dose (day 43) from mice immunized with three gonococcal OMV-based vaccines:

Each vaccine was given at a 10 μg dose and was adsorbed onto aluminum phosphate.
OMVs from an FA1090 1KO Δlpxl1 (1KO) strain with ΔLgtF mutation. The ΔLgtF mutation results in LOS with no sugars on its alpha or beta-chain.
OMVs from an FA1090 Δlpxl1, Δrmp (2KO) strain—OMVs display 2HexG+, 4HexG+ and 5HexG+ LOS structures.

OMVs from an FA1090 1KO Δlpxl1 (1KO) strain—OMVs display 2HexG− LOS structures. The pooled sera were analyzed by hSBA using a panel of gonococcal strains including the homologous FA1090 strain and 10 heterologous strains representative of the N. gonorrhoeae population based on the comparative genome analysis of a wide panel of circulating strains.

Results: Results are shown in Table 10 below

TABLE 10
hSBA titers measured against the indicated gonococcus strains on pooled sera from
CD1 mice immunized twice with OMV-FA1090-1KO ΔLgtF, OMV-FA1090-2KO, OMV-FA1090-1KO.

| | | WHO- | WHO- | | WHO- | WHO- | | | | | |
|---|---|---|---|---|---|---|---|---|---|---|---|
| | FA1090 | M | N | MS11 | F | G | BG27 | SK92 | GC14 | F62 | BG8 |
| OMV FA1090 | 4096 | 4096 | 64 | 128 | <16 | 256 | <16 | <32 | <32 | 128 | 256 |
| 1KO (Δlpxl1) + | | | | | | | | | | | |
| ΔlgtF | | | | | | | | | | | |
| OMV FA1090 | 16384 | 4096 | 16384 | 8192 | 256 | 16384 | 1024 | <32 | 4096 | >16384 | 16384 |
| 2KO (Δlpxl1, | | | | | | | | | | | |
| Δrmp) | | | | | | | | | | | |
| OMV FA1090 | 16384 | 8192 | 128 | 64 | 16 | 256 | <16 | <32 | <32 | 256 | 1024 |
| 1KO (Δlpxl1) | | | | | | | | | | | |

High hSBA titers were obtained for sera from mice immunized with OMVs from FA1090 2KO (Δlpxl1, Δrmp) against 9 out 11 gonococcus strains (low response for SK-92-679 and WHO-F).

OMVs obtained from an FA1090 strain comprising a ΔlgtF mutation were able to elicit good bactericidal antibodies against FA1090 and WHO-M while very low or negative hSBA titers were detected for the other heterologous strains. These results demonstrated that anti-LOS antibodies have a major role in SBA functional response against the majority of the heterologous strains.

Sera obtained from mice immunized with OMVs from a FA1090 1KO mutant (Δlpxl1) where the LOS structure was unusually highly truncated (2HexG−), presumably due to phase variation of the lgtG and lgtA genes, presented lower hSBA titers towards heterologous strains (except WHO-M) compared to OMVs obtained from an FA1090 2KO (Δlpxl1, Δrmp) strain containing both the β-chain and higher percentage of structures with a long α-chain (2HexG+, 4HexG+ and 5HexG+).

### Example 4: hSBA Titers—FA1090 ΔLgtF and ΔLgtG Mutants

Female CD1 outbred mice 7 weeks old (10/group) were immunized intraperitoneally 2 times at days 1, 29 with different lots of OMV-based vaccines (10 μg in 200 μl) adsorbed to Alum (3 mg/ml), or Alum alone (200 μl).

Pooled sera were collected two weeks after the second dose (day 43) from mice immunized with three vaccines:
OMVs from an FA1090 Δlpxl1, Δrmp (2KO) strain—2 different lots were tested. OMVs display 2HexG+, 4HexG+ and 5HexG+ LOS structures.
OMVs from an FA1090 Δlpxl1, Δrmp (2KO) strain with ΔlgtF mutation (OMVs display LOS with no sugars present on the alpha-chain or beta-chain).
OMVs from an FA1090 Δlpxl1, Δrmp (2KO) strain with ΔlgtG mutation (OMVs display short alpha-chain and no beta-chain (2HexG−, 3HexG−).

An alum-only negative control was also included. All sera were analyzed in duplicate with hSBA using a panel of gonococcal strains including the homologous FA1090 strain and 10 heterologous strains representative of the N. gonorrhoeae population based on the comparative genome analysis of a wide panel of circulating strains.

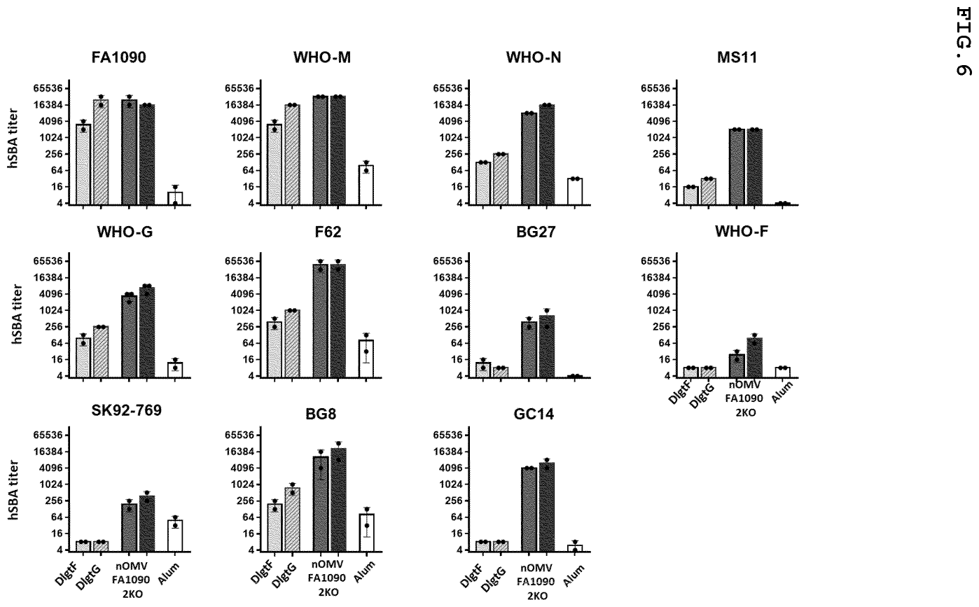

Results: Data is shown in FIG. 6—The results obtained immunizing mice with OMVs from the FA1090 2KO ΔlgtF strain and OMVs from the FA 1090 2KO ΔlgtG strain, confirmed what was observed in the previous in vivo study in which mice were immunized with OMVs from FA1090 1KO ΔlgtF. These two OMV vaccine preparations were able to elicit a high level of functional antibodies only against FA1090 and WHO-M while low hSBA titers were detected for the other heterologous strains, compared with hSBA titers obtained following immunization with OMVs obtained from an FA 1090 2KO strain (Δlpxl1, Δrmp). These results support the hypothesis that the majority of the heterologous strains are killed by anti-LOS antibodies.

### Example 3: Long Alpha-Chain LOS Structures are Essential for Elicitation of Functional SBA Titers in the Context of Neisseria gonorrhoeae OMV Vaccines

Animal immunization was conducted essentially as described above (see Example 2)

F62: OMVs obtained from an F62 2KO (Δlpxl, Δrmp) strain that presented a truncated LOS with a short α-chain (2HexG− and 3HexG−), elicited considerably lower hSBA titers in mice than OMVs obtained from an F62 1KO strain (3 Lots tested i.e. LOT 14, 15 and 20) which includes longer alpha-chain structures and the absence of the β-chain. It was concluded that LOS structures with long α-chain correlated with good bactericidal titers, while the presence of β-chain was not essential for elicitation of bactericidal antibodies by gonococcal OMV vaccines. Data is shown in Table 11 below:

TABLE 11

| | | Porb1b | | | | | Porb1a | |
|---|---|---|---|---|---|---|---|---|
| OMV Vaccine | LOS | FA1090 | WHO-M | MS11 | F62 | BG27 | SK92 | WHO-N |
| F62 OMV 2KO | 2HexG− | 53 | 96 | 8 | 320 | 6 | 5 | 107 |
| (Δplxl1, Δrmp) | 3HexG− | | | | | | | |
| F62 OMV 1KO | 2HexG− | 1536 | 2048 | 512 | 2730 | 384 | 5 | 3413 |
| (Δlpxl1) LOT 14 | 3HexG− | | | | | | | |
| | 4HexG− | | | | | | | |
| | 5HexG− | | | | | | | |
| F62 OMV 1KO | 2HexG− | 3072 | 6146 | 1024 | 8192 | 768 | 5 | 6827 |
| (Δlpxl1) LOT 15 | 3HexG− | | | | | | | |
| | 4HexG− | | | | | | | |
| | 5HexG− | | | | | | | |
| F62 OMV 1KO | 2HexG− | 6144 | 6144 | 1024 | 6827 | 768 | 4 | 6144 |
| (Δlpxl1) LOT 20 | 3HexG− | | | | | | | |
| | 4HexG− | | | | | | | |
| | 5HexG− | | | | | | | |
| Alum | N/A | 8 | 24 | 8 | 14 | 4 | 6 | 21 |

SK92: OMVs obtained from an SK92 1KO (Δlpxl1) strain possessing a single predominant LOS structure corresponding exactly to the minimal epitope recognized by mAb 2C7, (2HexG+), was not able to elicit a strong bactericidal response towards the majority of heterologous strains tested. The data confirmed that the presence of β-chain (2C7 epitope) without long α-chains did not correlate with high hSBA titers against heterologous strains. Data is shown in Table 12 below.

TABLE 12

| | | Porb1b | | | | | | |
|---|---|---|---|---|---|---|---|---|
| | | | WHO- | | | | Porb1a | |
| OMV Vaccine | LOS | FA1090 | M | MS11 | F62 | BG27 | SK92 | WHO-N |
| SK92 OMV 1KO | 2HexG+ | 192 | 256 | 24 | 1024 | 1280 | 9011 | 683 |
| (Δlpxl1) LOT 19 | | | | | | | | |
| SK92 OMV 1KO | 2HexG+ | 48 | 192 | 24 | 256 | 1536 | 18022 | 213 |
| (Δlpxl1) LOT 16 | | | | | | | | |
| Alum | N/A | 8 | 24 | 8 | 14 | 4 | 6 | 21 |

Together these data suggest that LOS structures having a long alpha-chain (4Hex/5Hex) correlate with high cross-bactericidal antibody titers in a majority of heterologous gonococcal strains.

### Example 4: OMVs Obtained from a Neisseria gonorrhoeae MS11 Strain which has been Genetically Fixed to Express Longer LOS Structures (Long α-Chain) Elicit Higher SBA Functional Titers Against Heterologous Strains that Those Expressing Short α-Chain Structures

Female CD1 outbred mice 7 weeks old (10/group) were immunized intraperitoneally 2 times at days 1, 29 with different lots of OMV-based vaccines adsorbed to Alum (3 mg/ml), or Alum alone (200 μl). Differently from the studies above, OMVs were normalized based on LOS content (1,5 nmol LOS in 200 μl).

Pooled sera were collected two weeks after the second dose (day43) from mice immunized with a panel of gonococcal OMV vaccines:
Group 1-8: 8 MS11 Δlpxl1 LOS mutant strains in which the expression of the four phase-variable lgt genes (lgtG, lgtA, lgtC and lgtD) was genetically fixed either on or off or deleted as shown in Table 13 below.

TABLE 13
Neisseria gonorrhoeae MS11 LOS mutant strains used in
this study. The expression of lgtG, lgtA, lgtC and lgtD were
fixed on or off and LOS was detoxified through lpxl1 KO.

| N. gonorrhoeae | | |
|---|---|---|
| strain | LOS phenotype | Description |
| 2-Hex/G⁻ | Gal-Glc-Hep I | lgtA-del |
| | Unsubstituted Hep II | lgtC-off |
| | | lgtG::kan |
| 2-Hex/G⁺ | Gal-Glc-Hep I | lgtA-del |
| | Gal-Glc-Hep II | lgtC-off |
| | | lgtG-on |
| 3-Hex/G⁻ | Gal-Gal-Glc-Hep I | lgtA-del |
| | Unsubstituted Hep II | lgtC-on |
| | | lgtG::kan |
| 3-Hex/G⁺ | Gal-Gal-Glc-Hep I | lgtA-del |
| | Gal-Glc-Hep II | lgtC-on |
| | | lgtG-on |
| 4-Hex/G⁻ | Gal-GlcNAc-Gal-Glc-Hep I | lgtA-on |
| | Unsubstituted Hep II | lgtD-del |
| | | lgtC-off |
| | | lgtG::kan |
| 4-Hex/G⁺ | Gal-GlcNAc-Gal-Glc-Hep I | lgtA-on |
| | Gal-Glc-Hep II | lgtD-del |
| | | lgtC-off |
| | | lgtG-on |
| 5-Hex/G⁻ | GalNAc-Gal-GlcNAc-Gal- | lgtA-on |
| | Glc-Hep I Unsubstituted | lgtD-on |
| | Hep II | lgtC-off |
| | | lgtG::kan |
| 5-Hex/G⁺ | GalNAc-Gal-GlcNAc-Gal- | lgtA-on |
| | Glc-Hep I | lgtD-on |
| | Gal-Glc-Hep II | lgtC-off |
| | | lgtG-on |

Group 9: OMVs from an FA1090 Δlpxl1, Δrmp (2KO) strain (OMVs display 2HexG+, 4HexG+, 5HexG+ LOS structures)
Group 10: Alum control

Samples were analyzed in duplicate or triplicate with hSBA using a panel of gonococcal strains including the homologous MS11 strain and 5 heterologous strains (FA1090, WHO-N, F62, WHO-G and SK92).

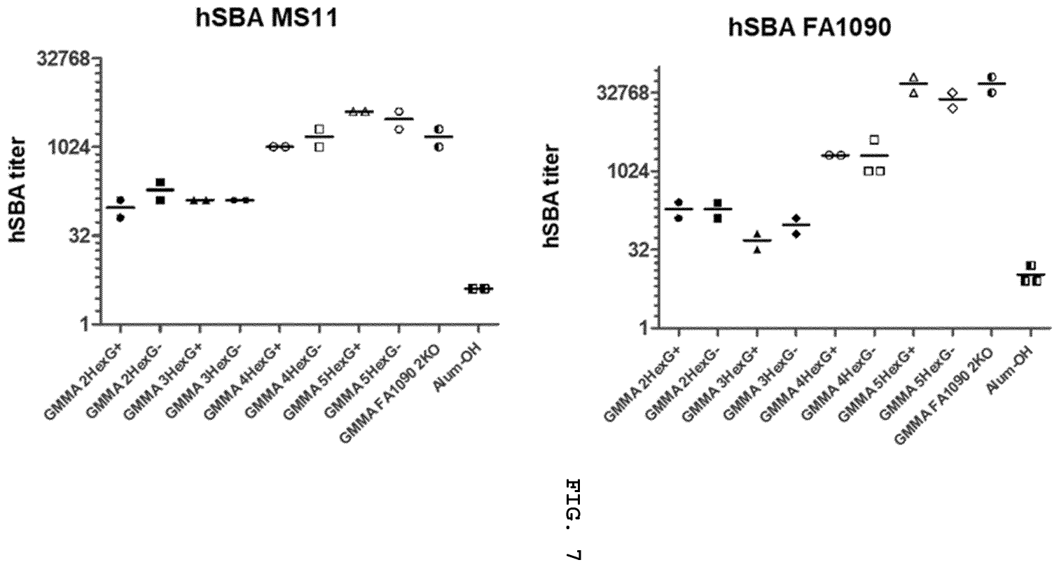

Results: Results are reported in Table 14 below and also in FIG. 7.

TABLE 14
hSBA titers measured against the indicated gonococcus strains on pooled
sera from CD1 mice immunized twice with the OMV based vaccines.

| Group | Vaccine | MS11 | FA1090 | WHO-N | F62 | WHO-G | SK92 |
|---|---|---|---|---|---|---|---|
| 1 | MS11 OMV | 512 | 128 | 1024 | 256 | 128 | 16 |
| | 2HexG+ Δlpxl1 | 64 | 256 | 512 | 1024 | 128 | |
| | | 128 | | | 256 | | |
| 2 | MS11 OMV | 512 | 128 | 512 | 256 | 256 | 32 |
| | 2HexG− Δlpxl1 | 128 | 256 | 256 | 512 | 256 | |
| | | 256 | | | 256 | | |
| 3 | MS11 OMV | 512 | 32 | 128 | 512 | 128 | 32 |
| | 3HexG+ Δlpxl1 | 128 | 64 | 128 | 512 | 64 | |
| | | 128 | | | 256 | | |
| 4 | MS11 OMV | 512 | 64 | 128 | 256 | 256 | 32 |
| | 3HexG− Δlpxl1 | 128 | 128 | 128 | 512 | 256 | |
| | | 128 | | | 256 | | |
| 5 | MS11 OMV | 8192 | 2048 | >8192 | 1024 | 1024 | 32 |
| | 4HexG+ Δlpxl1 | 1024 | >8192 | 4096 | 2048 | 4096 | |
| | | 1024 | 2048 | 2048 | 1024 | 2048 | |
| 6 | MS11 OMV | >16384 | 1024 | 4096 | 4096 | 2048 | 32 |
| | 4HexG− Δlpxl1 | 1024 | 4096 | 2048 | 2048 | >8192 | |
| | | 2048 | 1024 | 2048 | 2048 | 4096 | |
| 7 | MS11 OMV | >16384 | >8192 | >8192 | >8192 | >8192 | 32 |
| | 5HexG+ Δlpxl1 | 4096 | 65536 | 8192 | 131072 | 16384 | |
| | | 4096 | 32768 | 8192 | 65536 | 16384 | |
| 8 | MS11 OMV | >16384 | >8192 | >8192 | >8192 | >8192 | 32 |
| | 5HexG− Δlpxl1 | 2048 | 32768 | 8192 | 65536 | 8192 | |
| | | 4096 | 16384 | 4096 | 65536 | 4096 | |
| 9 | FA1090 OMV 2KO | 8192 | >8192 | >8192 | >8192 | 2048 | 32 |
| | (Δlpxl1, Δrmp) | 1024 | 65536 | 4096 | 32768 | 2048 | |
| | | 2048 | 32768 | 4096 | 16384 | 2048 | |
| 10 | Alum | <16 | 8 | 32 | 32 | 8 | 16 |
| | | <8 | 8 | 16 | 64 | 16 | |
| | | <8 | 16 | | 32 | 16 | |

The results obtained following immunization of mice with OMVVs exposing different LOS structures highlighted that pooled sera derived from mice immunized with OMVs with long alpha-chain LOS structures (4HexG+/G−; 5HexG+/G−) gave higher hSBA titers compared to the sera derived from mice immunized with the OMVs exposing shorter LOS structures (2HexG+/G−; 3HexG+/−) for 5 out of 6 tested N. gonorrhoeae strains. In particular the OMV preparations with longer alpha-chains were able to elicit a high level of functional antibodies against most of the tested strains except for SK92.

These results underline the higher impact of the anti-LOS antibodies elicited by OMVs with longer LOS structures (long α-chain) on the SBA functional response against 5 out of 6 tested strains. The presence of β-chain and in particular of 2C7 epitope (2HexG+ mutant) was clearly dispensable for elicitation of bactericidal antibodies, while the crucial parameter responsible for an increased hSBA titer was the length of α-chain.

### Example 5: OMVs Obtained from a Neisseria gonorrhoeae MS11 Strain which has been Genetically Fixed to Express Longer LOS Structures (Long α-Chain) Elicit Antibodies that Inhibit the Adhesion of Neisseria gonorrhoeae to Epithelial Cells

The MS11 strain used in this study was also genetically detoxified (i.e. lpxl1 mutants)

Sera obtained from the same in vivo study described in Example 4 above was tested in the BAI assay (for material and methods see Example 1).

### Results:

TABLE 15
shows the role of antisera directed against different LOS
structures on inhibition of Neisseria gonorrhoeae (FA1090)
adhesion to SV-HUC1 urethral epithelial cells:

| | | % Max Bacterial | Regression | Inhibition |
|---|---|---|---|---|
| Group | Antisera | Inhibition | p-value | Outcome |
| 1 | MS11 2-Hex/G⁺ | 26 | NA* | x |
| 2 | MS11 2-Hex/G⁻ | 38^{#} | 0.0044 | Undeter- |
| | | | | mined |
| 3 | MS11 3-Hex/G⁺ | 11 | NA* | x |
| 4 | MS11 3-Hex/G⁻ | 22 | NA* | x |
| 5 | MS11 4-Hex/G⁺ | 53 | <0.0001 | ✓ |
| 6 | MS11 4-Hex/G⁻ | 27 | NA* | x |
| 7 | MS11 5-Hex/G⁺ | 73 | <0.0001 | ✓ |
| 8 | MS11 5-Hex/G⁻ | 50 | <0.0008 | ✓ |
| 12 | Alum | 22/12 | NA* | x |

^{#}result of only a single repetition- statistical analysis not performed
*inhibition lower than 30% at all dilutions - regression p-value not computed.

TABLE 16
shows the role of antisera directed against different LOS
structures on inhibition of Neisseria gonorrhoeae (SK92)
adhesion to SV-HUC1 urethral epithelial cells:

| | | % Max Bacterial | Regression | Inhibition |
|---|---|---|---|---|
| Group | Antisera | Inhibition | p-value | Outcome |
| 1 | MS11 2-Hex/G⁺ | 60 | 0.02 | ✓ |
| 2 | MS11 2-Hex/G⁻ | 51# | 0.0004 | Undeter- |
| | | | | mined |
| 3 | MS11 3-Hex/G⁺ | 22 | N/A* | x |
| 4 | MS11 3-Hex/G⁻ | 59 | 0.0002 | ✓ |
| 5 | MS11 4-Hex/G⁺ | 42 | 0.0085 | ✓ |
| 6 | MS11 4-Hex/G⁻ | 53 | 0.0034 | ✓ |
| 7 | MS11 5-Hex/G⁺ | 55 | 0.0053 | ✓ |
| 8 | MS11 5-Hex/G⁻ | 58 | 0.0034 | ✓ |
| 12 | Alum | 14/18 | NA* | x |

^{#}result of only a single repetition- statistical analysis not performed
*inhibition lower than 30% at all dilutions - regression p-value not computed.

Conclusion: The results obtained following immunization of mice with MS11 OMVs exposing different LOS structures highlighted that sera obtained from mice immunized with OMVs with long alpha-chain LOS structures was able to inhibit the adhesion of FA1090 and SK92 gonococcus to urethral epithelial cells. For FA1090, only sera obtained from mice immunized with long alpha chain (4Hex and 5Hex) were capable of inhibiting adhesion. However, for SK92, sera obtained from mice immunized with OMVs having some shorter alpha chain LOS structures (e.g., 2HexG+) also induced antibodies capable to inhibiting adhesion to urethral epithelial cells. This is likely because the SK92 strain displays predominantly 2HexG+ LOS epitopes (see Table 9 above).

Overall, these results underline the higher impact of the anti-LOS antibodies elicited by OMVs with longer LOS structures (long α-chain) on inhibition of gonococcal adhesion.

### Example 6: Competitive hSBA Results

The competitive human Serum Bactericidal Assay (hSBA) is an assay able to measure functional antibodies that remain available after incubation with a specific competitor, thus enabling determination of the role of anti-LOS antibodies in SBA functional response.

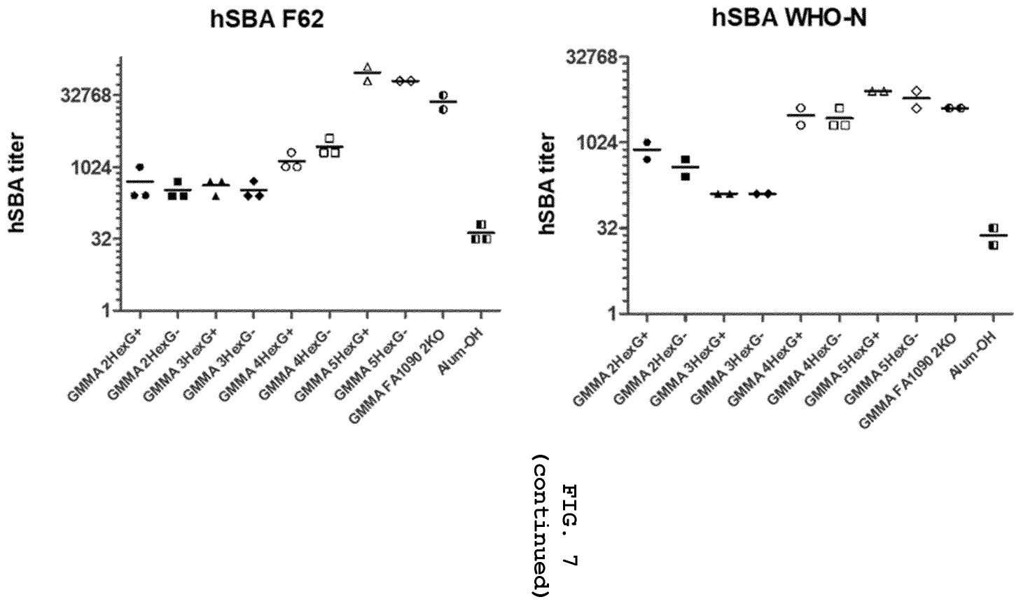

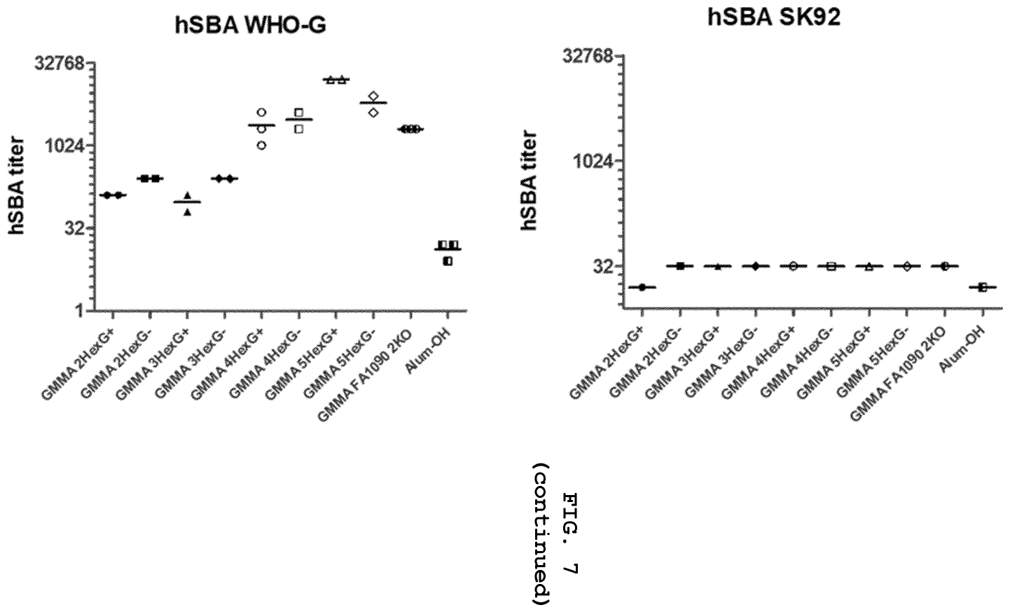

A pool of sera from mice immunized with OMVs isolated from an FA1090 Δlpxl1, Δrmp (2KO) strain (2HexG+, 4HexG+ and 5HexG+) that is known to elicit cross-bactericidal titers, was preincubated with 3 concentrations of 3 different inhibitors (expressed as LOS nmol/ml) as indicated in the FIG. 8.

The competitors were as follows:
LOS extracted from wild-type FA1090
OMVs (referred to as GMMA) obtained from FA1090 Δlpxl1 and ΔlgtF (i.e. no sugars on alpha or beta-chains)
OMVs from FA 1090 Δlpxl1, Δrmp (2KO) strain

The competitive hSBA experiment was conducted against four heterologous strains MS11, WHO-N, WHO-G and F62.

Results: Extracted LOS was able to abolish the hSBA titer of the test serum at least at the highest concentration tested on all the four strains as the homologous OMVs (lot32), while the GMMA (i.e. OMV) FA1090 1KO ΔLgtF did not compete with functional antibodies elicited by the OMV vaccine (OMVs obtained from FA1090 2KO Δlpxl1, Δrmp) that is known to elicit cross-bactericidal titers (See FIG. 8). These data confirm that the anti-LOS antibodies are responsible for the bactericidal activity of the sera obtained following administration of a gonococcal OMV vaccine.

In a second step using the same pooled sera (from mice immunized with OMVs which are known to elicit functional cross-bactericidal antibody titers) competitive hSBA experiments were run using as competitors OMVs generated from MS11 lgt mutant strains (Δlpxl1): nOMV 2HexG−, nOMV 2HexG+ nOMV 3HexG−, nOMV 3HexG+ nOMV 4HexG−, nOMV 4HexG+ nOMV 5HexG−, nOMV 5HexG+. Four heterologous gonococcal strains were tested.

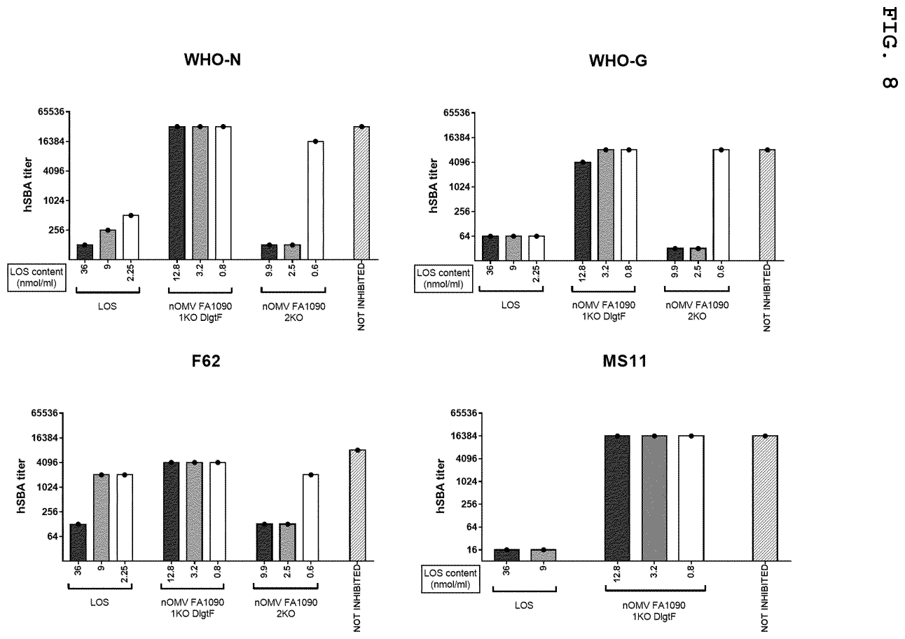

In FIG. 9 the obtained results are reported, showing that antibodies anti 4HexG+, 5HexG− and 5HexG+ LOS structures were bactericidal in hSBA for 3 out of 4 heterologous strains (MS11, WHO-N and WHO-G). These data confirm that the antibodies against LOS structures with a long α-chain are responsible for the bactericidal activity of the sera obtained following administration of OMVs obtained from the FA1090 2KO (Δlpxl1, Δrmp) strain. The hSBA titers are less inhibited when the experiment is conducted with F62 strain.

As a direct proof that a long α-chain LOS epitope is important for elicitation of functional responses in gonococcal OMV-based vaccines, we used MS11-derived LOS mutant strains. In these mutant strains, the lgt phase variable genes were locked ON or OFF, generating a single type of LOS structures on their bacterial surface. Native OMVs were produced and used in hSBA as competitors of a pool of sera from mice immunized with an OMV based vaccine which is known to induce cross-bactericidal titers. hSBA titers against different heterologous strains were drastically inhibited by OMVs with a long α-chain (4-5 sugar residues, structures 4HexG+, 5HexG− and 5HexG+) irrespectively of the presence of the β-chain and therefore of the 2C7 epitope. In contrast a structure containing only a short α-chain (2HexG+, the structure recognized by the 2C7 mAb with highest affinity) did not inhibit the functional activity.

The data confirmed that the functional antibodies were not the ones targeting the 2C7 epitope but instead those targeting an epitope containing the long α-chain.

### Example 7: Locking the PV of the Lgt Genes to Obtain a Long Alpha-Chain

Background: Phase variation of LOS glycan extensions is mediated by slipped-strand mispairing at homopolymeric tracts within the coding regions of the lipooligosaccharide glycosyl transferase (lgt) genes.
lgtA, lgtC, and lgtD modify glycan extensions from Hep I
lgtG permits glycan extensions from Hep II

To generate a gonococcal mutant that expresses long alpha-chain LOS structures, lgtA must be locked ON and lgtC must be locked OFF. The result of this is a gonococcal mutant expressing LOS having a long Hep I-extending alpha-chain (4HexG−/4HexG+/5HexG−/5HexG+ structures possible).

The expression of lgtD is either locked ON (promotes 5Hex structures) or left phase variable. Furthermore, the data shown in Examples 3-6 above indicate that the presence of a beta-chain is not strictly required for induction by OMV vaccines of cross-bactericidal antibody titres. As such, the expression of lgtG can either be left phase variable or locked ON.

Lgt genes were rendered non-phase variable by either locking expression ON or OFF (essentially as described in Chakraborti et al 2016). Locking ON the expression of the Lgt genes was conducted by modifying or removing the homopolymer. Locking OFF the expression of the Lgt genes was conducted either by gene deletion, insertional inactivation or introduction of stop codons.

As an alternative to making modifications within the locus of each individual lgt gene, an alternative strategy can be employed involving two steps.
1) deletion of the entire locus containing lgt genes (lgtA, lgtB, lgtC, lgtD and lgtE) and
2) replacement with a locus having whichever combination of LOS genes locked ON/OFF (e.g. lgtA locked ON, lgtC locked OFF).

For generation of a long alpha-chain, one strategy may be to replace the locus with the following combination of LOS genes locked ON/OFF.
lgtB and lgtE (non phase-variable thus retain as wild-type)
lgtA locked ON
lgtC locked OFF
lgtD locked ON

This choice ensures expression of LOS with a long alpha-chain (both 4Hex and 5Hex as lgtD does not have 100% efficiency) and would avoid the formation of the 3Hex structure. The lgtG gene is not modified since presence or absence of the beta-chain has no impact on the induction of cross-bactericidal antibodies.

What is claimed is:

1. An immunogenic composition comprising isolated gonococcal outer membrane vesicles (OMVs) obtained or obtainable from a genetically modified Neisseria gonorrhoeae bacterium said bacterium comprising genetic modification(s) that
a) render the phase variability of at least one lipooligosaccharide glycosyl transferase (lgt) gene(s) non-phase variable thus resulting in a bacterium that produces OMVs displaying lipooligosaccharide (LOS) glycan structures comprising an oligosaccharide alpha-chain elongating from Hep I having at least four hexose monosaccharides (4Hex or 5Hex) and,
b) detoxify the LOS.

2. The immunogenic composition of claim 1 wherein said genetic modification(s) that render the phase variability of at least one lgt gene(s) non-phase variable results in a bacterium that is incapable of producing OMVs displaying LOS glycan structures comprising an oligosaccharide alpha-chain elongating from Hep I having less than four hexose monosaccharides (2Hex or 3Hex).

3. The immunogenic composition of claim 1 wherein said genetic modification(s) that render the phase variability of said at least one lgt gene(s) non-phase variable either:
a. locks expression ON resulting in said at least one lgt genes(s) being constitutively expressed or,
b. locks expression OFF resulting in loss of expression of said at least one lgt gene(s).

4. The immunogenic composition of claim 3 wherein expression is locked ON by mutating the homopolymeric tract of said at least one lgt gene(s) such that said homopolymer is modified or removed, optionally wherein said homopolymer is modified or removed without altering the coding sequence for said at least one lgt gene.

5. The immunogenic composition of claim 3 wherein expression is locked OFF either:
i) by deleting said at least one lgt gene(s) or a portion thereof,
ii) by insertional inactivation of said at least one lgt gene(s), and/or
iii) by inserting stop codons within the open reading frame of said at least one lgt gene(s).

6. The immunogenic composition of claim 1 wherein said at least one lgt gene(s) comprise one, more or all of lgtA, lgtC, IgtD and/or IgtG.

7. The immunogenic composition of claim 1 wherein said genetic modification(s) that render the phase variability of at least one lgt gene(s) non-phase variable, renders the phase variability of:
(i) lgtA non-phase variable and locked ON resulting in lgtA being constitutively expressed, and
(ii) lgtC non-phase variable and locked OFF resulting in loss of lgtC expression.

8. The immunogenic composition of claim 1 wherein said genetic modification(s) to detoxify the LOS decreases or abolishes expression and/or function of the lipid A biosynthesis lauroyl acyltransferase (lpxl1) gene, lpxl1 mRNA and/or lpxl1 polypeptide.

9. The immunogenic composition of claim 1 wherein said genetically modified Neisseria gonorrhoeae bacterium comprises a yet further genetic modification, wherein said yet further genetic modification decreases or abolishes expression and/or function of the reduction modifiable protein (rmp) gene, rmp mRNA and/or rmp polypeptide.

10. The immunogenic composition of any preceding claim wherein the genetically modified Neisseria gonorrhoeae bacterium is derived from strain MS11, BG27, BG8, F62, FA1090, WHO-F, WHO-M, WHO-N, WHO-G, GC14, SK92-679 or GC_0817560.

11. An immunogenic composition comprising isolated gonococcal outer membrane vesicles (OMVs) wherein said composition substantially comprises OMVs that display lipooligosaccharide (LOS) glycan structures comprising an oligosaccharide alpha-chain elongating from Hep I having at least four hexose monosaccharides (4Hex or 5Hex) and wherein said LOS comprises a pentaacylated lipid A rather than a hexaacylated lipid A.

12. The immunogenic composition of claim 1 or claim 11 said composition comprising no detectable OMVs that display LOS glycan structures comprising an oligosaccharide alpha-chain elongating from Hep I having less than four hexose monosaccharides (2Hex or 3Hex).

13. The immunogenic composition of claim 12 wherein absence of mAb L1 and mAb 4C4 binding indicates that there are no detectable OMVs displaying LOS glycan structures comprising an oligosaccharide alpha-chain elongating from Hep I having less than four hexose monosaccharides (2Hex or 3Hex).

14. The immunogenic composition of claim 1 wherein over 80%, over 85%, over 90% or over 95% of the OMVs display LOS glycan structures comprising an oligosaccharide alpha-chain elongating from Hep I having at least four hexose monosaccharides (4Hex or 5Hex).

15. The immunogenic composition of claim 1 wherein said LOS is detoxified optionally wherein said detoxified LOS lacks the secondary lauroyl chain from the non-reducing end of the GlcN disaccharide.

16. The immunogenic composition of claim 1 wherein said oligosaccharide alpha-chain elongating from Hep I comprises either β-Gal-(1 á4)-β-GlcNAc-(1á3)-β-Gal-(1á4)-β-Glc-(1á4)-Hep I (4Hex) or β-GalNAc-(1á3)-β-Gal-(1á4)-β-GlcNAc-(1á3)-β-Gal-(1á&4)-β-Glc-(1á4)-Hep I (5Hex).

17. The immunogenic composition of claim 1 wherein said LOS glycan structures comprise Hep II and said Hep II either:
a) comprises its beta-oligosaccharide chain (4HexG+ or 5HexG+) or
b) does not comprise its beta-oligosaccharide chain (4HexG− or 5HexG−).

18. The immunogenic composition of claim 17 wherein Hep II does not comprise its beta oligosaccharide chain (4HexG− or 5HexG−).

19. The immunogenic composition of claim 1 wherein said isolated gonococcal OMVs comprise reduced levels or no detectable level of rmp polypeptide in comparison to an OMV from a wild-type Neisseria gonorrhoeae.

20. The immunogenic composition of claim 1 further comprising a pharmaceutically acceptable excipient.

21. The immunogenic composition of claim 1 further comprising an adjuvant, optionally an aluminum salt adjuvant, optionally aluminum hydroxide or aluminum phosphate.

22. A vaccine comprising the immunogenic composition of claim 1.

23. (canceled)

24. (canceled)

25. A method for the treatment or prevention of disease caused by N. gonorrhoeae in a subject in need thereof, said method comprising administering to said subject a therapeutically effective amount of the immunogenic composition of claim 1 or vaccine of claim 22.

26. A method for immunizing a subject in need thereof against N. gonorrhoeae, comprising administering an immunologically effective amount of the immunogenic composition of claim 1 or vaccine of claim 22.

27. A method for raising an immune response in a subject, comprising administering the immunogenic composition of claim 1 or vaccine of claim 22 to a subject.
